SEQUENCE LISTING (9 sequences)

SEQ ID NO: 1 - protein, 10 aa, synthetic construct
RGDSPGERCG

SEQ ID NO: 2 - protein, 11 aa, synthetic construct
ACDCRGDCFCG

SEQ ID NO: 3 - protein, 8 aa, synthetic construct
  MOD_RES 1: note = acetyl modified phenylalanine (modification at N-terminus)
  MOD_RES 8: note = amide-modified glycine (at C-terminus)
FKGGERCG

SEQ ID NO: 4 - protein, 8 aa, synthetic construct
FKGGERCG

SEQ ID NO: 5 - protein, 14 aa, synthetic construct
NQEQVSPLGGERCG

SEQ ID NO: 6 - protein, 6 aa, synthetic construct
GRGDSP

SEQ ID NO: 7 - protein, 5 aa, synthetic construct
  SITE 4: note = D-phenylalanine
  REGION 1..5: note = circular
RGDXV

SEQ ID NO: 8 - protein, 10 aa, synthetic construct
  SITE 4: note = D-phenylalanine
  SITE 9: note = D-phenylalanine
  REGION 1..5: note = circular
  REGION 6..10: note = circular
RGDXKRGDXC

SEQ ID NO: 9 - protein, 12 aa, synthetic construct
KACDCRGDCFCG

(19) United States
(12) Patent Application Publication
(10) Pub. No.: US 2026/0263652 A1
(43) Pub. Date: Sep. 10, 2026

(54) HYDROGEL FORMULATIONS AND METHODS OF USE THEREOF

(71) Applicant: Tempo Therapeutics, Inc., San Diego, CA (US)

(72) Inventors: Stephanie DESHAYES, Santee, CA (US); Westbrook WEAVER, San Diego, CA (US); Daria ANDRIYANOVA, San Diego, CA (US); Mitchell MCMAHON, San Diego, CA (US)

(21) Appl. No.: 19/425,166

(22) Filed: Dec. 18, 2025

Related U.S. Application Data
(63) Continuation of application No. PCT/US2024/035438, filed on Jun. 25, 2024.
(60) Provisional application No. 63/510,321, filed on Jun. 26, 2023.

(51) Int. Cl. A61L 17/00; A61L 27/18; A61L 27/52; A61L 27/56

(52) U.S. Cl. CPC A61L 17/005; A61L 27/18; A61L 27/52; A61L 27/56

(57) ABSTRACT

Provided herein are hydrogel formulations and methods for the use thereof. In certain aspects, the methods comprise use of hydrogel formulations in the strengthening of a suture line at a suture line site.

### CROSS-REFERENCE

This application is a continuation application of International Application No. PCT/US2024/035438, filed Jun. 25, 2024, which claims the benefit of U.S. Provisional Application No. 63/510,321, filed Jun. 26, 2023, which application is incorporated herein by reference in its entirety.

### INCORPORATION BY REFERENCE OF SEQUENCE LISTING

The present application is being filed along with a Sequence Listing in electronic format. The Sequence Listing is provided as a file entitled 48469-709.301_SL.xml, created Dec. 17, 2025, which is 11,869 bytes in size. The information in the electronic format of the Sequence Listing is incorporated by reference in its entirety.

### SUMMARY

In certain aspects, described herein is a method of delivering a hydrogel formulation to a site of a suture line in a subject, the method comprising: delivering to the suture line site of the subject the hydrogel formulation that anneals in situ to form a porous covalently stabilized scaffold, wherein endogenous cells infiltrate the porous covalently stabilized scaffold to form a cell matrix over the suture line site of the subject. In some embodiments, the suture line was an incision. In some embodiments, the incision was a surgical incision. In some embodiments, the surgical incision was an abdominal fascial incision. In some embodiments, the abdominal fascial incision was an abdominal wall midline or transverse incision of the fascia. In some embodiments, the abdominal incision was a result of a surgery performed on the subject comprising: a gastro-intestinal cancer procedure, a hysterectomy, an ovarian cancer procedure, a spinal fusion, an abdominal trauma surgery, or a combination thereof. In some embodiments, the delivering comprises delivering the hydrogel formulation directly over the suture line after the suture line is sutured. In some embodiments, the delivering comprises delivering the hydrogel formulation into the suture line while the suture line is being sutured. In some embodiments, the delivering comprises delivering the hydrogel formulation into the suture line after the incision is sutured. In some embodiments, the delivering comprises delivering the hydrogel formulation into the suture line while the suture line is being sutured and on top of the suture line after the suture line is sutured. In some embodiments, the hydrogel begins to anneal on top of the suture line before the dermis and subcutis of the subject is sutured. In some embodiments, the hydrogel anneals on top of the suture line for at least about 10 minutes before the dermis and subcutis of the subject is sutured. In some embodiments, the delivering comprises releasing the hydrogel formulation from a syringe. In some embodiments, the method further comprises strengthening the suture line at the suture line site by the cell matrix formed over the suture line site. In some embodiments, the strengthening the suture line is characterized by increasing a mechanical tensile strength of the suture line as compared to a reference suture line at an otherwise identical suture line site that was sutured without the delivery of the hydrogel formulation. In some embodiments, the increasing the mechanical tensile strength of the suture line is characterized by the formation of an amount or a type of collagen mimicking endogenous tissue at the suture line site. In some embodiments, the collagen is formed at the suture line site by at least about 28 days after suturing of the suture line. In some embodiments, the collagen is formed in and around the porous covalently stabilized scaffold. In some embodiments, the type of collagen comprises Type I collagen, Type III collagen, or a combination thereof. In some embodiments, Type I collagen is present with Type III collagen in a ratio of less than or equal to about 10:1, less than or equal to about 6:1, or less than or equal to about 5:1. In some embodiments, the increasing the mechanical tensile strength of the suture line is characterized by increasing the yield stress of the suture line. In some embodiments, the yield stress is calculated from a stress versus strain curve measured using a tensile test (e.g., on an Instron). In some embodiments, the suture line comprises a yield stress of at least about 3.0 N/mm² to about 6.0 N/mm² at least about 42 days after suturing of the suture line. In some embodiments, the increasing the mechanical tensile strength of the suture line is characterized by increasing the toughness of the suture line. In some embodiments, the toughness is measured as an area under the curve of a stress versus strain curve to fracture using a tensile test (e.g., on an Instron). In some embodiments, the suture line comprises a toughness of at least about 60.0 millijoules per millimeter cubed (mJ/mm³) at least about 42 days after suturing of the suture line. In some embodiments, the suture line comprises a toughness of at least about 25.0 mJ/mm³ to about 100.0 mJ/mm³ at least about 42 days after suturing of the suture line. In some embodiments, the increasing the mechanical tensile strength of the suture line is characterized by increasing a percent recovery of the suture line. In some embodiments, the percent recovery of the suture line is greater than or equal to about 40% increased as compared to a reference suture line sutured without the delivery of the hydrogel formulation. In some embodiments, the percent recovery of the suture line is at least about 20% to about 60% increased as compared to a reference suture line sutured without the delivery of the hydrogel formulation. In some embodiments, the increasing the mechanical tensile strength of the suture line is characterized by increasing the yield strain of the suture line. In some embodiments, the hydrogel formulation becomes integrated with the suture line site in less than or equal to about 14 days following suturing of the suture line. In some embodiments, integration is characterized by new tissue formation in and around the porous covalently stabilized scaffold. In some embodiments, the cell matrix forms new tissue at the suture line site of the subject before complete degradation of the porous covalently stabilized scaffold. In some embodiments, the new tissue is characterized by having (i) mature vascularization, (ii) a characteristic of surrounding tissue at the suture line site, (iii) an amount or a type of collagen mimicking endogenous tissue at the suture line site (iii) or a combination thereof. In some embodiments, the characteristic of the surrounding tissue at the suture line site comprises functionally differentiated cell types from the surrounding tissue. In some embodiments: (i) the new tissue forms, and (ii) the porous covalently stabilized scaffold completely degrades by at least about 42 days after suturing the suture line. In some embodiments, the new tissue is formed in addition to any tissue formed at the suture line site due to sutures alone. In some embodiments, additional new tissue continues to form at the suture line site for at least about 42 days after suturing of the suture line. In some embodiments, the new tissue is formed above (e.g., superficial to) the suture line site and deep to subcutaneous tissue. In some embodiments, the new tissue is stromal like tissue with non-aligned collagen bundles. In some embodiments, the porous covalently stabilized scaffold strengthens the suture line at the suture line site of the subject while minimizing a foreign body response in the subject. In some embodiments, the foreign body response is characterized by causing harm to the subject. In some embodiments, the harm is characterized by causing: chronic inflammation, granuloma formation, scar tissue formation, adhesion formation, nodule formation, swelling, pain, or any combination thereof. In some embodiments, the harm is caused at the suture line site. In some embodiments, the porous covalently stabilized scaffold is effective to strengthen the suture line at the suture line site of the subject while minimizing a foreign body response in the subject when the foreign body response is measured by detecting an amount of granulomas at the suture line site with histological analysis and comparing the amount of granulomas at the suture line site with a reference suture line site that does not contain the hydrogel formulation. In some embodiments, the porous covalently stabilized scaffold is effective to strengthen the suture line at the suture line site of the subject while minimizing a foreign body response in the subject when the foreign body response is measured by detecting an amount of scar tissue at the suture line site with histological analysis and comparing the amount of scar tissue at the suture line site with a reference suture line site that does not contain the hydrogel formulation. In some embodiments, the porous covalently stabilized scaffold is effective to strengthen the suture line at the suture line site of the subject while minimizing a foreign body response in the subject when the foreign body response is measured by detecting an amount of nodules at the suture line site with histological analysis and comparing the amount of nodules at the suture line site with a reference suture line site that does not contain the hydrogel formulation. In some embodiments, the porous covalently stabilized scaffold is effective to strengthen the suture line at the suture line site of the subject while minimizing a foreign body response in the subject when the foreign body response is measured by detecting chronic inflammation at the suture line site with histological analysis. In some embodiments, the porous covalently stabilized scaffold is effective to strengthen the suture line at the suture line site of the subject while minimizing a foreign body response in the subject when the foreign body response is measured by a presence of one or more types of macrophages at the suture line site of the subject. In some embodiments, the one or more types of macrophages comprise type 1 macrophages and type 2 macrophages. In some embodiments, the type 1 macrophages are pro-inflammatory. In some embodiments, the type 2 macrophages are pro-regenerative. In some embodiments, the porous covalently stabilized scaffold is effective to form more type 2 macrophages than type 1 macrophages. In some embodiments, the cell matrix comprises an amount or a type of collagen mimicking endogenous tissue at the suture line site. In some embodiments, the collagen is formed at the suture line site by at least about 28 days after suturing of the suture line. In some embodiments, the collagen is formed in and around the porous covalently stabilized scaffold. In some embodiments, the type of collagen comprises Type I collagen, Type III collagen, or a combination thereof. In some embodiments, Type I collagen is present with Type III collagen in a ratio of less than or equal to about 10:1, less than or equal to about 6:1, or less than or equal to about 5:1. In some embodiments, at least part of the suture line site comprises elastin following degradation of the porous covalently stabilized scaffold at the suture line site. In some embodiments, the porous covalently stabilized scaffold comprises a pH of about 8. In some embodiments, the porous covalently stabilized scaffold comprises an elastic compressive modulus of at least about 1500 Pascals (Pa) after the annealing reaction. In some embodiments, the hydrogel formulation comprises microgel particles comprising a cross-linked 4-arm poly(ethylene glycol) (PEG) vinyl sulfone hydrogel polymer cross-linked with a matrix metalloprotease (MMP)-degradable crosslinker, one or more K-peptides and Q-peptides, and a cell-adhesive peptide. In some embodiments, the microgel particles are present in a suspension comprising the microgel particles and water, and the microgel particles are present in the suspension at a volume fraction of at least 90%. In some embodiments, the hydrogel formulation further comprises PEG-dithiol, wherein the microgel particles undergo an annealing reaction to form a porous covalently stabilized scaffold after exposure to the PEG-dithiol. In some embodiments, the PEG-dithiol comprises a molecular weight of at least about 0.5 kilodaltons (kDa) to about 10 kDa. In some embodiments, the PEG-dithiol comprises a molecular weight of at least about 3.4 kDa. In some embodiments, the PEG-dithiol is present in the hydrogel system in a molar concentration of at least about 0.02 millimolar (mM) to about 1.0 mM. In some embodiments, the PEG-dithiol is present in the hydrogel system in a molar concentration of at least about 0.2 mM.

In certain aspects, described herein is a hydrogel system, comprising: microgel particles comprising a cross-linked 4-arm poly(ethylene glycol) (PEG) vinyl sulfone hydrogel polymer cross-linked with a matrix metalloprotease (MMP)-degradable crosslinker, one or more K-peptides and Q-peptides, and a cell-adhesive peptide, wherein the microgel particles are present in a suspension comprising the microgel particles and water, wherein the microgel particles are present in the suspension at a volume fraction of at least 90%; and PEG-dithiol, wherein the PEG-dithiol comprises a molecular weight of at least about 0.5 kilodaltons (kDa) and wherein the microgel particles undergo an annealing reaction to form a porous covalently stabilized scaffold after exposure to the PEG-dithiol. In certain aspects, described herein is a hydrogel system, comprising: microgel particles comprising a cross-linked 4-arm poly(ethylene glycol) (PEG) vinyl sulfone hydrogel polymer cross-linked with a matrix metalloprotease (MMP)-degradable crosslinker, one or more K-peptides and Q-peptides, and a cell-adhesive peptide, wherein the microgel particles are present in a suspension comprising the microgel particles and water, and the microgel particles are present in the suspension at a volume fraction of at least 90%; and PEG-dithiol, wherein the microgel particles undergo an annealing reaction to form a porous covalently stabilized scaffold after exposure to the PEG-dithiol, wherein the PEG-dithiol comprises is present in the hydrogel system in a molar concentration of at least about 0.02 millimolar (mM) to about 1.0 mM. In some embodiments, the microgel particles are present in the suspension at a volume fraction of at least 90%. In some embodiments, the PEG-dithiol and the 4-arm PEG vinyl sulfone are present to provide a ratio of thiol to vinyl sulfone of less than about 1.0. In some embodiments, the PEG-dithiol comprises a molecular weight of at least about 3.4 kDa. In some embodiments, the PEG-dithiol is present in the hydrogel system in a molar concentration of at least about 0.2 mM. In some embodiments, the microgel particles are spherical. In some embodiments, the microgel particles comprise microspheres. In some embodiments, the microgel particles comprise diameters comprising 5 μm to 1000 μm, between 50 μm to 1000 μm, or between 70 μm to 150 μm. In some embodiments, the microgel particles comprise an elastic compressive modulus of at least about 500 Pascals (Pa) before the annealing reaction. In some embodiments, the microgel particles comprise an elastic compressive modulus of at least about 1500 Pascals (Pa) after the annealing reaction. In some embodiments, the porous covalently stabilized scaffold comprises pores comprising a median pore diameter of about 5 μm and above. In some embodiments, the pores comprise a median pore diameter of about 10 μm to about 35 μm. In some embodiments, the one or more cell adhesive peptides comprises an RGD peptide. In some embodiments, the microgel particles comprise a polydispersity of no more than 0.1. In some embodiments, the polydispersity is calculated based on a standard deviation and mean size of the particles (e.g., PDI=(SD/mean)\hat{ }2). In some embodiments, the hydrogel formulation further comprises a buffer, wherein the buffer comprises: a phosphate buffer, a 4-(2-hydroxyethyl)-1-piperazineethanesulfonic acid (HEPES) buffer, or an acetate buffer, or any combination thereof. In some embodiments, the annealing reaction comprises a covalent synthesizing reaction. In some embodiments, the covalent synthesizing reaction comprises a Michael addition or a pseudo-Michael addition reaction. In some embodiments, the vinyl sulfone of the 4-arm PEG vinyl sulfone is a Michael acceptor in the Michael addition or pseudo-Michael addition reaction. In some embodiments, the thiol of the PEG-dithiol is a Michael donor in the Michael addition or pseudo-Michael addition reaction. In some embodiments, described herein is a hydrogel formulation comprising the hydrogel system described herein in a suspension, wherein the suspension comprises a buffer. In some embodiments, the formulation is formulated for administration to a subject. In some embodiments, the formulation is formulated for administration at a suture line of a suture line of the subject. In some embodiments, the administration minimizes a foreign body response in the subject. In some embodiments, the formulation comprises a dose volume of about 0.01 mL to about 20 mL. In some embodiments, described herein is a delivery device comprising: a body comprising the hydrogel system described herein or the hydrogel formulation described herein; and an applicator in fluidic communication with the body, wherein the delivery device is sterile. In some embodiments, the delivery device is a syringe or needle.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1A depicts the normalized yield strength versus abdominal position, according to some embodiments herein.

FIG. 1B depicts the normalized yield strength per rabbit, according to some embodiments herein.

FIG. 1C depicts the normalized yield strength at different storage conditions, according to some embodiments herein.

FIG. 2A depicts the zipper method of applying microgel annealed particles (MAP), according to some embodiments herein.

FIG. 2B depicts the post-suture method of applying MAP, according to some embodiments herein.

FIG. 2C depicts the two-step method of applying MAP, according to some embodiments herein.

FIG. 2D depicts the one-step method of applying MAP, according to some embodiments herein.

FIG. 2E is a schematic of midline incision and MAP application on top of the incision (procedure #3) with offset dermal incision and closure, according to some embodiments herein.

FIG. 3A depicts yield strength measured by a linearly actuated mechanical analyzer (e.g. an Instron 3342 system) for non-injured tissue, for injured tissue treated with sutures alone and for injured tissue treated with MAP (Formulation 1 at 80%) and sutures, 7 and 14 days after treatment, according to some embodiments herein.

FIG. 3B depicts the ratio of yield strength of injured tissue to non-injured tissue after treatment with sutures alone or with MAP (Formulation 1 at 80%) and sutures, according to some embodiments herein. Formulation 1 was applied within the suture during suturing and on top of the incision (procedure #4). Normalization was done per the width of tissue section.

FIG. 4A depicts representative histology images of the abdominal wall 7 days after incision and treatment with MAP (Formulation 1), according to some embodiments herein.

FIG. 4B depicts representative histology images of the abdominal wall 14 days after incision and treatment with MAP (Formulation 1), according to some embodiments herein. Formulation 1 was applied within the suture during suturing and on top of the incision (procedure #4). Formulation 1 appears to be present on top and bottom of the tissue but not within the incision and not right on top of the incision but rather on the sides.

FIG. 5A depicts representative histology images of abdominal wall 14 days after incision and treatment with Formulation 1 (VF=80%), according to some embodiments herein. Here, the dermis was closed offset from the midline, and Formulation 1 was applied only on top of the incision (procedure #3). Rabbits #23 and #20.

FIG. 5B depicts the normalized yield strength measured by Instron for injured tissue treated with sutures alone and for injured tissue treated with MAP (Formulation 1 at 80%) and sutures, 14 days after treatment, according to some embodiments herein.

FIG. 5C depicts the ratio of yield strength of injured tissue to non-injured tissue after treatment with sutures alone or with MAP (Formulation 1 at 80%) and sutures, according to some embodiments herein. Formulation 1 was applied on top of the incision (procedure #3) and the dermis was closed offset from the midline. Normalization was done per the width of tissue section.

FIG. 6A depicts stress for different MAP formulations, according to some embodiments herein. N represent the number of strips tested (3 strips per rabbit were collected). Here the dermis was closed offset from the midline. Normalization was done per surface are=width of tissue section×thickness (since all sections were assumed to have the same thickness, then dividing by the thickness does not really change the data trend between yield strength in N/mm and stress in N/mm²).

FIG. 6B depicts representative histology images of abdominal wall 14 days after incision and treatment with Formulation 5 (VF=100%), according to some embodiments herein. Here, the dermis was closed offset from the midline, and Formulation 5 was applied only on top of the incision (procedure #3). Rabbits #26 and #27.

FIG. 6C depicts representative histology images of abdominal wall 14 days after incision and treatment with Formulation 4 (annealed with PRP at VF=90%), according to some embodiments herein. Here, the dermis was closed offset from the midline, and Formulation 4 was applied only on top of the incision (procedure #3). Rabbits #36 and #37.

FIG. 6D depicts representative histology images of abdominal wall 14 days after incision and treatment with Formulation 3 (annealed with PEG-dithiol crosslinker at VF=90%), according to some embodiments herein. Here, the dermis was closed offset from the midline, and Formulation 4 was applied only on top of the incision (procedure #3). Rabbits #30 and #34.

FIG. 6E depicts representative histology images of abdominal wall 14 days after incision and treatment with Formulation 3 (annealed with PEG-dithiol crosslinker at VF=90%), according to some embodiments herein. Here, the dermis was closed offset from the midline, and Formulation 4 was applied within the incision after suturing and on top of the incision (procedure #5). Rabbits #31 and #33.

FIG. 6F depicts stress for different MAP formulations and different application methods, according to some embodiments herein. N represent the number of strips tested (3 strips per rabbit were collected).

FIG. 6G depicts the MAP formulations used in this experiment at 14 days after surgery.

FIG. 7A depicts the yield stress of abdominal incisions over time when Formulation 3 was administered compared to sutures alone, according to some embodiments herein.

FIG. 7B depicts the percent of recovery at 42 days from abdominal incisions when Formulation 3 was administered compared to sutures alone, according to some embodiments herein.

FIG. 7C depicts the yield stress of incisions where Formulation 3 was applied at 14, 28, and 42 days after surgery, according to some embodiments herein.

FIG. 7D depicts the yield stress of incisions where Formulation 3 was not applied at 14, 28, and 42 days after surgery, according to some embodiments herein.

FIG. 7E depicts a representative stress extension curve for abdominal incisions where Formulation 3 was applied compared to sutures alone, according to some embodiments herein.

FIG. 7F depicts incisions site toughness at 28 and 42 days of incisions where Formulation 3 was applied and incisions where Formulation 3 was not applied, according to some embodiments herein.

FIG. 8A depicts Hematoxylin and Eosin (H&E) histology images of incisions after 14 days, according to some embodiments herein. The number on each image indicates the rabbit number and the section number of the tissue.

FIG. 8B depicts Picrosirius red histology images of incisions after 14 days, according to some embodiments herein. The number on each image indicates the rabbit number and the section number of the tissue.

FIG. 8C depicts detailed H&E and Picrosirius red histology images of incisions after 14 days, according to some embodiments herein. The number on each image indicates the rabbit number and the section number of the tissue.

FIG. 8D depicts a histology image of an incision treated with Formulation 3 after 14 days, according to some embodiments herein.

FIG. 8E depicts detailed H&E and Picrosirius red histology images of incisions treated with Formulation 3 after 14 days, according to some embodiments herein. The number on each image indicates the rabbit number and the section number of the tissue as indicated in FIG. 8D. Arrows indicate the following features: a. cellular infiltration into the scaffold, b engraftment of scaffold to site by cell growth, c. scaffold structure mostly intact, d. neovascularization of the scaffold, e. new collagen deposition over and within the scaffold.

FIG. 8F depicts detailed H&E and Picrosirius red histology images of incisions after 28 days, according to some embodiments herein. The number on each image indicates the rabbit number and the section number of the tissue.

FIG. 8G depicts a histology image of an incision treated with Formulation 3 after 28 days, according to some embodiments herein.

FIG. 8H depicts detailed H&E and Picrosirius red histology images of incisions treated with Formulation 3 after 28 days, according to some embodiments herein. The number on each image indicates the rabbit number and the section number of the tissue as depicted in FIG. 8G. Arrows indicate the following features: a. increased cellular infiltration, b. recently degraded scaffold structure, c. mature vascularization of tissue and intact scaffold, d. significant collagen deposition around and within the scaffold.

FIG. 8I depicts detailed H&E and Picrosirius red histology images of incisions after 42 days, according to some embodiments herein. The number on each image indicates the rabbit number and the section number of the tissue.

FIG. 8J depicts a histology image of an incision treated with Formulation 3 after 42 days, according to some embodiments herein.

FIG. 8K depicts detailed H&E and Picrosirius red histology images of incisions treated with Formulation 3 after 42 days, according to some embodiments herein. The number on each image indicates the rabbit number and the section number of the tissue as depicted on FIG. 8J. Arrows indicate the following features: a complete cellular infiltration into the scaffold, b. majority of scaffold degraded and volume replaced with tissue and macrophages, c. mature vascularization of the new tissue, d. significant collage deposition in tissue with both fibrillar and “basket weave” morphology.

FIG. 8L depicts H&E histology images of incisions over time. The number on each image indicates the rabbit number and the section number of the tissue, according to some embodiments herein.

FIG. 8M depicts detailed histology images of incisions over time. The number on each image indicates the rabbit number and the section number of the tissue, according to some embodiments herein.

### DETAILED DESCRIPTION

Midline abdominal incisions are commonly used in multiple human patient populations. The procedure associated with these incisions is called a midline laparotomy. These midline laparotomies are used in millions of patients every year including gastro-intestinal cancer procedures, hysterectomies and other ovarian cancer procedures, spinal fusions, and abdominal trauma surgeries. Currently, the incision made to access the abdomen re-opens in 30% of patients within a 24-month window after surgery, when these patients have certain comorbidities including diabetes, Body Mass Index (BMI)>25, or are undergoing primary tumor resections. This re-opening causes major complications and even death in some patients.

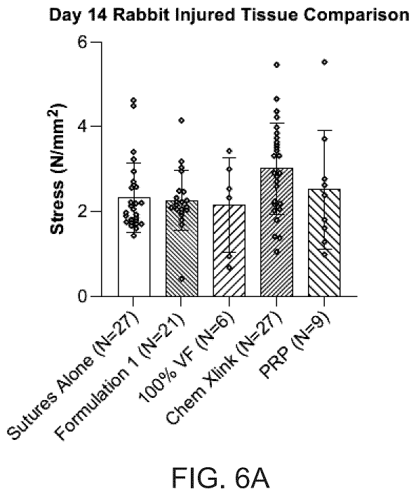

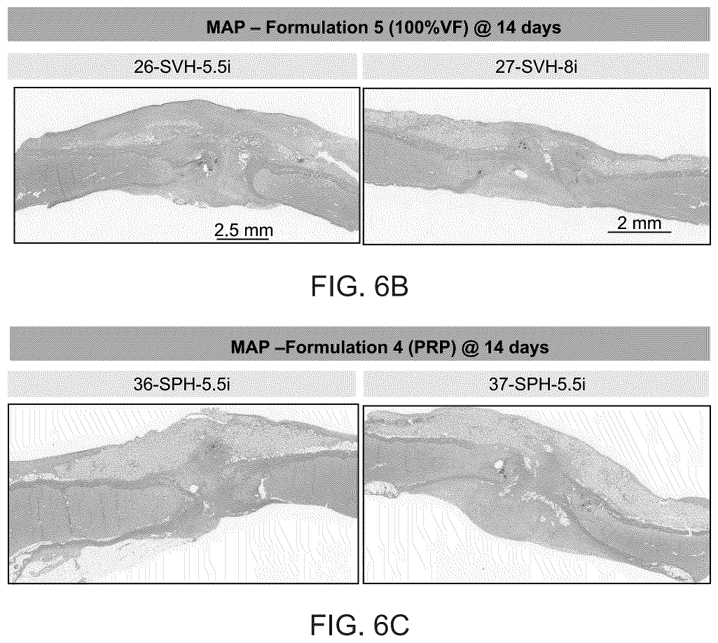

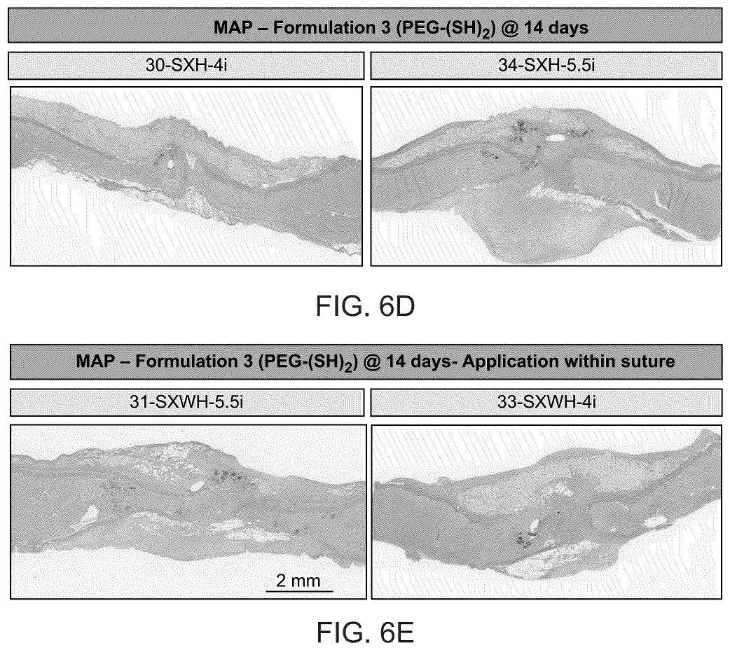

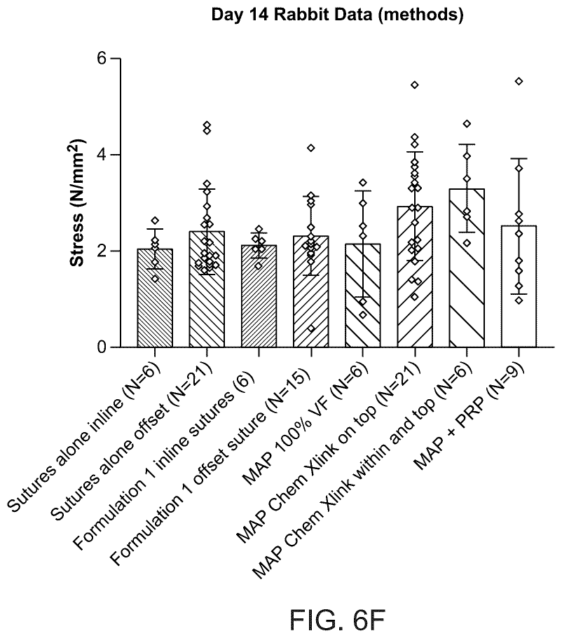

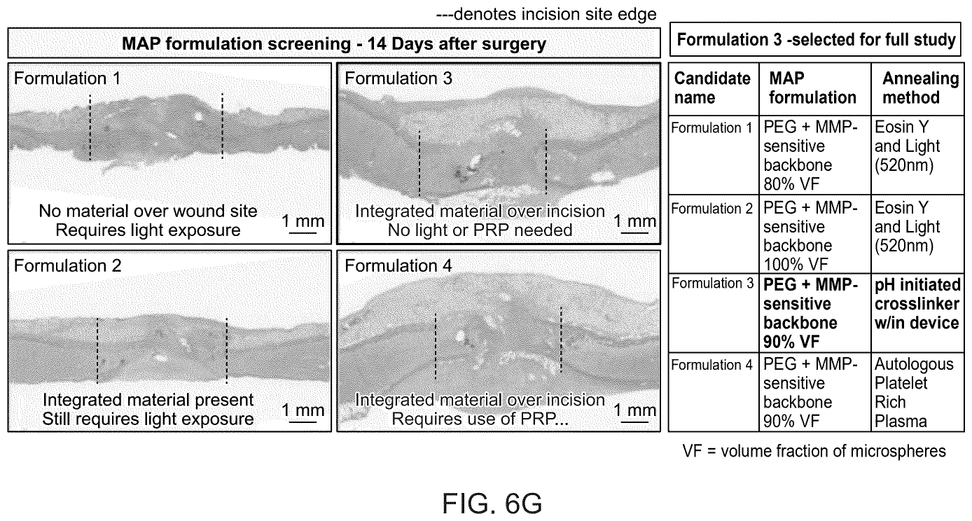

In certain aspects, described herein are systems and compositions to make sites of suture lines, including midline abdominal incisions, stronger through regenerative healing and will have the potential to greatly reduce this rate of post-surgical failure and reduce morbidity and mortality in this large patient population. In some embodiments, the systems and compositions described herein comprise one or more hydrogel formulations provided in FIG. 6G. In some embodiments, the systems and compositions comprise microgel particles comprising a 4-arm poly(ethylene glycol) (PEG) vinyl sulfone hydrogel polymer cross-linked with a matrix metalloprotease (MMP)-degradable crosslinker, one or more K-peptides and Q-peptides, and a cell-adhesive peptide. In some embodiments, the systems and compositions comprise PEG-dithiol comprising a molecular weight of at least about 0.5 kilodaltons to about 10 kilodaltons.

In certain aspects, described herein are methods of delivering the hydrogel formulations described herein to a site of a suture line of a subject. In some embodiments, the hydrogel formulation anneals in situ to form a porous covalently stabilized scaffold. In some embodiments, endogenous cells infiltrate the porous covalently stabilized scaffold to from a cell matrix over the incision. In some embodiments, the hydrogel formulation is delivered to a subject with a device disclosed herein, such as a syringe or a needle. In some embodiments, the methods and/or hydrogel formulations are optimized accordingly to various embodiments herein for a given application (e.g., to achieve a desired viscosity upon application, and mechanical properties upon annealing in situ).

### Systems

Hydrogel systems are gel-like substances that may be useful for certain therapeutic applications. For therapeutic applications, hydrogel systems can strengthen the site of a suture line when used as an adjunct to suture closure. As an example, strengthening the site of a suture line may be characterized by: increasing a mechanical tensile strength of the suture line, increasing the yield stress of the suture line, increasing the toughness of the suture line, increasing a percent recovery of the suture line, increasing the mechanical tensile strength of the suture line, or enabling collagen formation at the suture line site. In some embodiments, the strengthening the site of a suture line is effective to help prevent the re-opening of a suture line. In some embodiments, the systems and compositions described herein comprise one or more hydrogel formulations provided in FIG. 6G.

In certain aspects, described herein is a hydrogel system or a hydrogel formulation comprising microgel particles as described herein. In some embodiments, the hydrogel system comprises poly(ethylene glycol) (PEG)-dithiol. In some embodiments, the hydrogel system comprises microgel particles comprising a cross-linked 4-arm PEG vinyl sulfone hydrogel polymer cross-linked with a MMP-degradable crosslinker, one or more K-peptides and Q-peptides, and a cell-adhesive peptide, wherein the microgel particles are present in a suspension comprising the microgel particles and water, and the microgel particles are present in the suspension at a volume fraction of at least 90%; and PEG-dithiol, wherein the microgel particles undergo an annealing reaction to form a porous covalently stabilized scaffold after exposure to the PEG-dithiol, wherein the PEG-dithiol comprises a molecular weight of at least about 0.5 kilodaltons (kDa) to about 10 kDa and is present in the hydrogel system in a molar concentration of at least about 0.2 millimolar (mM).

### Microgel Particles

Disclosed herein, in some embodiments, are hydrogel formulations comprising a plurality of microgel particles. In some embodiments, the plurality of microgel particles is formed by crosslinking one or more reagents and raw materials together in accordance with various embodiments herein. The plurality of microgel particles may be in a slurry suitable for delivery to the subject by injection or by application on top of the suture line. Following delivery of the slurry to the subject at a tissue site, the microgel particles undergo an annealing reaction to form a porous covalently stabilized scaffold in situ. The porous covalently stabilized scaffold comprises the annealed microgel particles and may be referred to interchangeably as the “annealed particles,” “annealed microgel particles,” or “annealed scaffold.” In some embodiments, the porous covalent stabilized scaffold forms in a manner such that pores form between the microgel particles of the covalently stabilized scaffold. In some embodiments, when the hydrogel formulation is administered to a subject, the covalently stabilized scaffold enables growth of a cell matrix in situ at or around the tissue site of the subject that forms new tissue even after the covalently stabilized scaffold is completely degraded. In some embodiments, covalently stabilized scaffold enables growth of a cell matrix in situ at or around the tissue site of the subject that forms new tissue before the covalently stabilized scaffold begins to degrade (e.g., degradation of the covalently stabilized scaffold is not required for the cell matrix and new tissue to form). In some embodiments, the new tissue that is formed is endogenous tissue of the subject. In some embodiments, the new tissue is characterized as having mature vascularization, a characteristic of surrounding tissue at the tissue site, or a combination thereof. In some embodiments, the characteristic of the surrounding tissue at the tissue site comprises functionally differentiated cell types from the surrounding tissue. In some embodiments, the characteristic of the surrounding tissue at the suture line site comprises an amount or a type of collagen mimicking endogenous tissue at the suture line site.

In some embodiments, the microgel particles are spherical. In some embodiments, the microgel particles are spheroidal. In some embodiments, the microgel particles are substantially spherical or substantially spheroidal. In some embodiments, the microgel particles comprise microspheres. The microgel particles may have a substantially uniform shape so as to produce pores when adjacent microgel particles are in contact with each other. Other shapes of microgel particles are contemplated, including, without limitation, oblate, prolate, round-particles, granular particles, flake particles, or 3D geometric shapes.

In some embodiments, the microparticles may have a diameter or dimension (e.g., length, width, height, axis). In some embodiments, the microgel particles comprise diameters or dimensions comprising 0.1 micrometers (μm) to 1000 μm. In some embodiments, the microgel particles comprise diameters or dimensions comprising 5 micrometers (μm) to 1000 μm. In some embodiments, the diameters or dimensions comprise between 50 μm to 1000 μm. In some embodiments, the diameters or dimensions comprise between 80 μm to 140 μm. In some embodiments, the diameters or dimensions comprise between 70 μm to 150 μm In some embodiments, the diameters comprise greater than or equal to about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, or 1100 μm. In some embodiments, the diameters comprise less than or equal to about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, or 1100 μm. In some embodiments, the diameters or dimensions comprise a range of from about 5 μm to about 1100 μm. In some embodiments, the diameters or dimensions comprise a range of from about 10 μm to about 1090 μm. In some embodiments, the diameters or dimensions comprise a range of from about 15 μm to about 1080 μm. In some embodiments, the diameters or dimensions comprise a range of from about 20 μm to about 1070 μm. In some embodiments, the diameters or dimensions comprise a range of from about 25 μm to about 1060 μm. In some embodiments, the diameters or dimensions comprise a range of from about 30 μm to about 1050 μm. In some embodiments, the diameters or dimensions comprise a range of from about 35 μm to about 1040 μm. In some embodiments, the diameters or dimensions comprise a range of from about 40 μm to about 1030 μm. In some embodiments, the diameters or dimensions comprise a range of from about 45 μm to about 1020 μm. In some embodiments, the diameters or dimensions comprise a range of from about 50 μm to about 1010 μm. In some embodiments, the diameters or dimensions comprise a range of from about 55 μm to about 1000 μm. In some embodiments, the diameters or dimensions comprise a range of from about 60 μm to about 990 μm. In some embodiments, the diameters or dimensions comprise a range of from about 65 μm to about 980 μm. In some embodiments, the diameters or dimensions comprise a range of from about 70 μm to about 970 μm. In some embodiments, the diameters or dimensions comprise a range of from about 75 μm to about 960 μm. In some embodiments, the diameters or dimensions comprise a range of from about 80 μm to about 950 μm. In some embodiments, the diameters or dimensions comprise a range of from about 85 μm to about 940 μm. In some embodiments, the diameters or dimensions comprise a range of from about 90 μm to about 930 μm. In some embodiments, the diameters or dimensions comprise a range of from about 95 μm to about 920 μm. In some embodiments, the diameters or dimensions comprise a range of from about 100 μm to about 910 μm. In some embodiments, the diameters or dimensions comprise a range of from about 110 μm to about 900 μm. In some embodiments, the diameters or dimensions comprise a range of from about 120 μm to about 890 μm. In some embodiments, the diameters or dimensions comprise a range of from about 130 μm to about 880 μm. In some embodiments, the diameters or dimensions comprise a range of from about 140 μm to about 870 μm. In some embodiments, the diameters or dimensions comprise a range of from about 150 μm to about 860 μm. In some embodiments, the diameters or dimensions comprise a range of from about 160 μm to about 850 μm. In some embodiments, the diameters or dimensions comprise a range of from about 170 μm to about 840 μm. In some embodiments, the diameters or dimensions comprise a range of from about 180 μm to about 830 μm. In some embodiments, the diameters or dimensions comprise a range of from about 190 μm to about 820 μm. In some embodiments, the diameters or dimensions comprise a range of from about 200 μm to about 810 μm. In some embodiments, the diameters or dimensions comprise a range of from about 210 μm to about 800 μm. In some embodiments, the diameters or dimensions comprise a range of from about 220 μm to about 790 μm. In some embodiments, the diameters or dimensions comprise a range of from about 230 μm to about 780 μm. In some embodiments, the diameters or dimensions comprise a range of from about 240 μm to about 770 μm. In some embodiments, the diameters or dimensions comprise a range of from about 250 μm to about 760 μm. In some embodiments, the diameters or dimensions comprise a range of from about 260 μm to about 750 μm. In some embodiments, the diameters or dimensions comprise a range of from about 270 μm to about 740 μm. In some embodiments, the diameters or dimensions comprise a range of from about 280 μm to about 730 μm. In some embodiments, the diameters or dimensions comprise a range of from about 290 μm to about 720 μm. In some embodiments, the diameters or dimensions comprise a range of from about 300 μm to about 710 μm. In some embodiments, the diameters or dimensions comprise a range of from about 310 μm to about 700 μm. In some embodiments, the diameters or dimensions comprise a range of from about 320 μm to about 690 μm. In some embodiments, the diameters or dimensions comprise a range of from about 330 μm to about 680 μm. In some embodiments, the diameters or dimensions comprise a range of from about 340 μm to about 670 μm. In some embodiments, the diameters or dimensions comprise a range of from about 350 μm to about 660 μm. In some embodiments, the diameters or dimensions comprise a range of from about 360 μm to about 650 μm. In some embodiments, the diameters or dimensions comprise a range of from about 370 μm to about 640 μm. In some embodiments, the diameters or dimensions comprise a range of from about 380 μm to about 630 μm. In some embodiments, the diameters or dimensions comprise a range of from about 390 μm to about 620 μm. In some embodiments, the diameters or dimensions comprise a range of from about 400 μm to about 610 μm. In some embodiments, the diameters or dimensions comprise a range of from about 410 μm to about 600 μm. In some embodiments, the diameters or dimensions comprise a range of from about 420 μm to about 590 μm. In some embodiments, the diameters or dimensions comprise a range of from about 430 μm to about 580 μm. In some embodiments, the diameters or dimensions comprise a range of from about 440 μm to about 570 μm. In some embodiments, the diameters or dimensions comprise a range of from about 450 μm to about 560 μm. In some embodiments, the diameters or dimensions comprise a range of from about 460 μm to about 550 μm. In some embodiments, the diameters or dimensions comprise a range of from about 470 μm to about 540 μm. In some embodiments, the diameters or dimensions comprise a range of from about 480 μm to about 530 μm. In some embodiments, the diameters or dimensions comprise a range of from about 490 μm to about 520 μm. In some embodiments, the diameters or dimensions comprise a range of from about 500 μm to about 510 μm.

The microgel particles may have an average diameter or dimension of about 10 μm. The microgel particles may have an average diameter or dimension of about 15 μm. The microgel particles may have an average diameter or dimension of about 25 μm. The microgel particles may have a diameter or dimension of about 50 μm. The microgel particles may have an average diameter or dimension of about 100 μm. The microgel particles may have an average diameter or dimension of about 150 μm. The microgel particles may have an average diameter or dimension of about 200 μm. The microgel particles may have a diameter or dimension within the range of about 10 μm to about 500 μm. The microgel particles may have a diameter or dimension within the range of about 10 μm to about 200 μm. The microgel particles may have a diameter or dimension within the range of about 15 μm to about 200 μm. The microgel particles may have a diameter or dimension within the range of about 15 μm to about 150 μm. The microgel particles may have a diameter or dimension within the range of about 30 μm to about 100 μm. The microgel particles may have an average diameter or dimension of 10 μm. The microgel particles may have an average diameter or dimension of 15 μm. The microgel particles may have an average diameter or dimension of 25 μm. The microgel particles may have a diameter or dimension of 50 μm. The microgel particles may have an average diameter or dimension of 100 μm. The microgel particles may have an average diameter or dimension of 150 μm.

The microgel particles may have an average diameter or dimension of 200 μm. The microgel particles may have a diameter or dimension within the range of 10 μm to 500 μm. The microgel particles may have a diameter or dimension within the range of 10 μm to 200 μm. The microgel particles may have a diameter or dimension within the range of 15 μm to 200 μm. The microgel particles may have a diameter or dimension within the range of 15 μm to 150 μm. The microgel particles may have a diameter or dimension within the range of 30 μm to 100 μm. In some embodiments, the diameter of the microgel particle may be measured by (1) measuring the area of a microgel particle, (2) solving for the radius of the microgel particle using the equation for the area of a circle (i.e., A=pi*r\hat{ }2) and (3) solving for the diameter by multiplying the radius by 2 (i.e., D=2*r).

In some embodiments, the microgel particles comprise one or more cell adhesive peptides. In some embodiments, the cell adhesive peptide comprises at least a portion of an extracellular matrix protein. In some embodiments, the cell adhesive peptide comprises at least a portion of a collagen. In some embodiments, the cell adhesive peptide comprises at least a portion of a fibronectin. In some embodiments, the cell adhesive peptide comprises an integrin. In some embodiments, the adhesive peptide comprises a ligand to a receptor expressed on the cell. In some embodiments, the adhesive peptide comprises a cluster of differentiation (CD) protein. In some embodiments, the adhesive peptide comprises a naturally-occurring peptide. In some embodiments, the adhesive peptide comprises a synthetic peptide. In some embodiments, the cell adhesive peptide may be homologous to the naturally-occurring peptide. In some embodiments, the cell adhesive peptide comprises at least about 70% homologous to a naturally-occurring peptide. In some embodiments, the cell adhesive peptide is at least about 80% homologous to a naturally-occurring peptide. In some embodiments, the cell adhesive peptide comprises at least about 90% homology to a naturally-occurring peptide. In some embodiments, the cell adhesive peptide comprises at least 70% homology to a naturally-occurring peptide. In some embodiments, the cell adhesive peptide comprises at least 80% homology to a naturally-occurring peptide. In some embodiments, the cell adhesive peptide comprises at least 90% homology to a naturally-occurring peptide. In some embodiments, the cell adhesive peptide may be coupled to a surface of the microgel particle. In some embodiments, the cell adhesive peptides are grafted to the surface of the microgel particle. In some embodiments, the coupling may comprise one or more chemical bonds. In some embodiments, the one or more chemical bonds is one or more covalent bonds.

By way of non-limiting examples, the cell adhesive peptide may comprise an RGD peptide. In some embodiments, the RGD peptide comprises RGDSPGERCG (SEQ ID NO: 1). In some embodiments, the RGD peptide comprises ACDCRGDCFCG (SEQ ID NO: 2). In some embodiments, the RGD peptide comprises GRGDSP (SEQ ID NO: 6). In some embodiments, the RGD peptide comprises cyclo(Arg-Gly-Asp-DPhe-Val) (SEQ ID NO: 7). In some embodiments, the RGD peptide comprises cyclo(Arg-Gly-Asp-DPhe-Lys) cyclo(Arg-Gly-Asp-DPhe-Cys) (SEQ ID NO: 8). In some embodiments, the RGD peptide comprises KACDCRGDCFCG (SEQ ID NO: 9). In some embodiments, the RGD peptide comprises an amino acid sequence that is about 75% identical to an amino acid sequence provided in SEQ ID NO: 1. In some embodiments, the RGD peptide comprises an amino acid sequence that is about 85% identical to an amino acid sequence provided in SEQ ID NO: 1. In some embodiments, the RGD peptide comprises an amino acid sequence that is about 95% identical to an amino acid sequence provided in SEQ ID NO: 1. In some embodiments, the RGD peptide comprises an amino acid sequence that is about 75% identical to an amino acid sequence provided in SEQ ID NO: 2. In some embodiments, the RGD peptide comprises an amino acid sequence that is about 85% identical to an amino acid sequence provided in SEQ ID NO: 2. In some embodiments, the RGD peptide comprises an amino acid sequence that is about 95% identical to an amino acid sequence provided in SEQ ID NO: 2. In some embodiments, the RGD peptide comprises an amino acid sequence that is about 75% identical to an amino acid sequence provided in SEQ ID NO: 6. In some embodiments, the RGD peptide comprises an amino acid sequence that is about 85% identical to an amino acid sequence provided in SEQ ID NO: 6. In some embodiments, the RGD peptide comprises an amino acid sequence that is about 95% identical to an amino acid sequence provided in SEQ ID NO: 6. In some embodiments, the RGD peptide comprises an amino acid sequence that is about 75% identical to an amino acid sequence provided in SEQ ID NO: 7. In some embodiments, the RGD peptide comprises an amino acid sequence that is about 85% identical to an amino acid sequence provided in SEQ ID NO: 7. In some embodiments, the RGD peptide comprises an amino acid sequence that is about 95% identical to an amino acid sequence provided in SEQ ID NO: 7. In some embodiments, the RGD peptide comprises an amino acid sequence that is about 75% identical to an amino acid sequence provided in SEQ ID NO: 8. In some embodiments, the RGD peptide comprises an amino acid sequence that is about 85% identical to an amino acid sequence provided in SEQ ID NO: 8. In some embodiments, the RGD peptide comprises an amino acid sequence that is about 95% identical to an amino acid sequence provided in SEQ ID NO: 8. In some embodiments, the RGD peptide comprises an amino acid sequence that is about 75% identical to an amino acid sequence provided in SEQ ID NO: 9. In some embodiments, the RGD peptide comprises an amino acid sequence that is about 85% identical to an amino acid sequence provided in SEQ ID NO: 9. In some embodiments, the RGD peptide comprises an amino acid sequence that is about 95% identical to an amino acid sequence provided in SEQ ID NO: 9. In some embodiments, the RGD peptide comprises an amino acid sequence that is about 50%, 55%, 60%, 65%, 70%, 75%, 80%, 85%, 90%, 95%, 96%, 97%, 98% or 99% identical to an amino acid sequence provided in any one of SEQ ID NOS: 1-2 or 6-9. In some embodiments, the RGD peptide is provided in Moral MEG, Siahaan T J., et. al., which is hereby incorporated by reference in its entirety. In some embodiments, the RGD peptide is modified to improve conjugation of the RGD peptide to a substrate, such as for example, a microgel particle disclosed herein. Non-limiting examples of modifications include addition of a cysteine residue, addition of a linker with a thiol group on one end and an amine group on the other end, a linker with a thiol group on one end and a carboxylic acid group on the other end. In some embodiments, the RGD peptide comprises a modification on either terminus (e.g., C terminus, N terminus). In some embodiments, the modification is within a flanking sequence of the RGD motif within the RGD sequence.

Cell adhesive peptides may comprise an amino acid. In some embodiments, a cell adhesive peptide comprises a K peptide. In some embodiments, the one or more K peptides comprises an amino acid sequence provided in Ac-FKGGERCG-NH2 (SEQ ID NO: 3). In some embodiments, the K peptides comprises an amino acid sequence comprising FKGGERCG (SEQ ID NO: 4). In some embodiments, the K peptide comprises an amino acid sequence that is about 75% identical to an amino acid sequence provided in SEQ ID NO: 3. In some embodiments, the K peptide comprises an amino acid sequence that is about 85% identical to an amino acid sequence provided in SEQ ID NO: 3. In some embodiments, the K peptide comprises an amino acid sequence that is about 95% identical to an amino acid sequence provided in SEQ ID NO: 3. In some embodiments, the K peptide comprises an amino acid sequence that is about 50%, 55%, 60%, 65%, 70%, 75%, 80%, 85%, 90%, 95%, 96%, 97%, 98%, or 99% identical to an amino acid sequence provided in SEQ ID NO: 3. In some embodiments, the K peptide comprises an amino acid sequence that is about 75% identical to an amino acid sequence provided in SEQ ID NO: 4. In some embodiments, the K peptide comprises an amino acid sequence that is about 85% identical to an amino acid sequence provided in SEQ ID NO: 4. In some embodiments, the K peptide comprises an amino acid sequence that is about 95% identical to an amino acid sequence provided in SEQ ID NO: 4. In some embodiments, the K peptide comprises an amino acid sequence that is about 50%, 55%, 60%, 65%, 70%, 75%, 80%, 85%, 90%, 95%, 96%, 97%, 98%, or 99% identical to an amino acid sequence provided in SEQ ID NO: 4. In some embodiments, the cell adhesive peptide comprises a Q peptide. In some embodiments, the Q peptide comprises an amino acid sequence provided in NQEQVSPLGGERCG (SEQ ID NO: 5). In some embodiments, the Q peptide comprises an amino acid sequence that is about 75% identical to an amino acid sequence provided in SEQ ID NO: 5. In some embodiments, the Q peptide comprises an amino acid sequence that is about 85% identical to an amino acid sequence provided in SEQ ID NO: 5. In some embodiments, the Q peptide comprises an amino acid sequence that is about 95% identical to an amino acid sequence provided in SEQ ID NO: 5. In some embodiments, the Q peptide comprises an amino acid sequence that is about 50%, 55%, 60%, 65%, 70%, 75%, 80%, 85%, 90%, 95%, 96%, 97%, 98%, or 99% identical to an amino acid sequence provided in SEQ ID NO: 5.

In some embodiments, the microgel particles comprise a polymer. In some embodiments, the polymer is or comprises a polymer backbone. In some embodiments, the polymer backbone of the polymer is comprised of the main chain of the polymer (e.g., within a substance, the polymer making up a larger proportion of the substance as compared to other polymers in the substance). In some embodiments, the main chain is a linear chain in the polymer to which any other chain may be regarded as being pendant. In some embodiments, the polymer is or comprises a co-polymer. In some embodiments, a co-polymer comprises a polymer chain comprising two or more different monomers in substantially equal proportions. In some embodiments, the polymer is capable of crosslinking and holding large amounts of water forming a water insoluble hydrogel. In some embodiments, the polymer is a synthetic polymer. Non-limiting examples of polymer include poly(ethylene glycol), polyacrylamide, and polymethacrylate. In some embodiments, the polymer may comprise a hydrophilic polymer, amphiphilic polymer, synthetic polymer, or a copolymer of hydrophobic and hydrophilic polymers (e.g., poly(ethylene glycol) (PEG), poly(propylene glycol), or poly(hydroxyethyl methacrylate). In some embodiments, the polymer may be made from any synthetic polymer (e.g., PEG) capable of forming a hydrogel. In some embodiments, the polymer may comprise a functional group (e.g. vinyl) at least one end of the polymer (e.g. poly(ethylene glycol) methacrylate). In some embodiments, the polymer may comprise a functional group (e.g. vinyl) incorporated into the polymer backbone (e.g. poly(methacrylate)). In some embodiments, the polymer may comprise a vinyl polymer, such as, for example: poly(ethylene glycol) acrylate, poly(ethylene glycol) methacrylate, poly(ethylene glycol) vinyl sulfone, poly(ethylene glycol) maleimide, poly(ethylene glycol) norbornene, and poly(ethylene glycol) allyl. In some embodiments, the polymer may comprise a polyacrylamide or a polymethacrylate. In some embodiments, the polymer may comprise a polyester, a polyamide, a polyurethane, or a mixture or copolymer thereof. In some embodiments, the polymer is or comprises poly(ethylene) glycol (PEG). In some embodiments, the polymer consists of PEG. In some embodiments, the microgel particles comprise two or more types of polymers (e.g., polymers made of different materials). In some embodiments, the two or more types of polymers comprise PLA and PEG. In some embodiments, the two or more types of polymers comprise PMMA and PEG. In some embodiments, the two or more types of polymers comprise a polymer comprising a functional group and a polymer that does not comprise a functional group. In some embodiments, the microgel particles comprise three or more types of polymers each independently made of a material selected from the group consisting of a hydrophilic polymer, amphiphilic polymer, synthetic polymer. In some embodiments, the microgel particles comprise three or more types of polymers each independently made of a material selected from poly(ethylene glycol) (PEG), poly(lactic acid) (PLA), poly(propylene glycol), poly(hydroxyethyl methacrylate), or modified versions of any of these. In some embodiments, when the microgel particles comprise two or more types of polymers, the ratio of each polymer included in the microgel particles may vary. In some embodiments, the polymer comprises a 4-armed PEG vinyl sulfone.

In some embodiments, the microgel particles comprise a polymer comprising PEG or a modified version thereof. In some embodiments, the microgel particles comprise a MMP-degradable crosslinker. In some embodiments, the MMP-degradable crosslinker crosslinks the polymer within a microgel particle.

In some embodiments, the polymer (e.g., PEG vinyl sulfone) may be present in the microgel particles, the hydrogel formulation containing the microgel particles, the resulting covalently stabilized scaffold, or any combination thereof at about 0.5% weight (% wt) to about a 50% wt. In some embodiments, the polymer may be present in the microgel particles, the hydrogel formulation containing the microgel particles, the resulting covalently stabilized scaffold, or any combination thereof at greater than or equal to about 1% wt, 5% wt, 10% wt, 15% wt, 20% wt, 25% wt, 30% wt, 35% wt, 40% wt, 45% wt, or 50% wt. In some embodiments, the polymer may be present at less than or equal to about 1% wt, 5% wt, 10% wt, 15% wt, 20% wt, 25% wt, 30% wt, 35% wt, 40% wt, 45% wt, or 50% wt. In some embodiments, the polymer may be present at greater than or equal to about 0.10% wt, 0.2% wt, 0.3% wt, 0.4% wt, 0.5% wt, 0.6% wt, 0.7% wt, 0.8% wt, 0.9% wt, 1% wt, 1.1% wt, 1.2% wt, 1.3% wt, 1.4% wt, 1.5% wt, 1.6% wt, 1.7% wt, 1.8% wt, 1.9% wt, 2.0% wt, 2.5% wt, 3% wt, 4% wt or 5% wt. In some embodiments, the polymer may be present at less than or equal to about 0.1% wt, 0.2% wt, 0.3% wt, 0.4% wt, 0.5% wt, 0.6% wt, 0.7% wt, 0.8% wt, 0.9% wt, 1% wt, 1.1% wt, 1.2% wt, 1.3% wt, 1.4% wt, 1.5% wt, 1.6% wt, 1.7% wt, 1.8% wt, 1.9% wt, 2.0% wt, 2.5% wt, 3% wt, 4% wt or 5% wt. In some embodiments, the polymer may be present at about 5% weight (% wt) to about a 45% wt. In some embodiments, the polymer may be present at about 10% weight (% wt) to about a 40% wt. In some embodiments, the polymer may be present at about 15% weight (% wt) to about a 35% wt. In some embodiments, the polymer may be present at about 20% weight (% wt) to about a 30% wt. In some embodiments, the polymer may be present at about 0.1% wt to about 1.5% wt. In some embodiments, the polymer may be present at about 0.5% wt.

In some embodiments, the polymer is modified relative to an otherwise identical polymer that does not contain a modification. In some embodiments, the modified polymer comprises modified PEG, such as for example PEG modified to contain a thiol or derivative thereof. Non-limiting modifications include thiolation of the terminal alcohol group on the PEG polymer. In some embodiments, the modified polymer comprises a PEG vinyl sulfone. In some embodiments, the modified polymer comprises a 4-arm PEG vinyl sulfone (4-arm PEG-VS).

In some embodiments, the molecular weights of the polymers may have an effect on the properties of the microgel particles, the hydrogel formulation containing the microgel particles, the resulting covalently stabilized scaffold, or any combination thereof.

In some embodiments, the polymer (e.g., 4-arm PEG-VS) comprises a molecular weight of about 1 kilodalton (kDa) to about 60 kDa. In some embodiments, the polymer (e.g., 4-arm PEG-VS) comprises a molecular weight of about 0.5, 1, 2, 3, 4, 5, 6, 7, 8, 9, or 10 kDa. In some embodiments, the polymer (e.g., 4-arm PEG-VS) comprises a molecular weight of about of 1 kilodalton (kDa) to 1000 kDa. In some embodiments, the polymer (e.g., 4-arm PEG-VS) comprises a molecular weight of greater than or equal to about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, or 1100 kDa. In some embodiments, the PEF comprises a molecular weight of at least about 3.4 kDa. In some embodiments, the polymer (e.g., 4-arm PEG-VS) comprises a molecular weight of less than or equal to about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, or 1100 kDa. In some embodiments, the polymer (e.g., 4-arm PEG-VS) comprises a molecular weight range of from about 5 kDa to about 1100 kDa. In some embodiments, the polymer (e.g., 4-arm PEG-VS) comprises a molecular weight range of from about 10 kDa to about 1090 kDa. In some embodiments, the polymer (e.g., 4-arm PEG-VS) comprises a molecular weight range of from about 15 kDa to about 1080 kDa. In some embodiments, the polymer (e.g., 4-arm PEG-VS) comprises a molecular weight range of from about 20 kDa to about 1070 kDa. In some embodiments, the polymer (e.g., 4-arm PEG-VS) comprises a molecular weight range of from about 25 kDa to about 1060 kDa. In some embodiments, the polymer (e.g., 4-arm PEG-VS) comprises a molecular weight range of from about 30 kDa to about 1050 kDa. In some embodiments, the polymer (e.g., 4-arm PEG-VS) comprises a molecular weight range of from about 35 kDa to about 1040 kDa. In some embodiments, the polymer (e.g., 4-arm PEG-VS) comprises a molecular weight range of from about 40 kDa to about 1030 kDa. In some embodiments, the polymer (e.g., 4-arm PEG-VS) comprises a molecular weight range of from about 45 kDa to about 1020 kDa. In some embodiments, the polymer (e.g., 4-arm PEG-VS) comprises a molecular weight range of from about 50 kDa to about 1010 kDa. In some embodiments, the polymer (e.g., 4-arm PEG-VS) comprises a molecular weight range of from about 55 kDa to about 1000 kDa. In some embodiments, the polymer (e.g., 4-arm PEG-VS) comprises a molecular weight range of from about 60 kDa to about 990 kDa. In some embodiments, the polymer (e.g., 4-arm PEG-VS) comprises a molecular weight range of from about 65 kDa to about 980 kDa. In some embodiments, the polymer (e.g., 4-arm PEG-VS) comprises a molecular weight range of from about 70 kDa to about 970 kDa. In some embodiments, the polymer (e.g., 4-arm PEG-VS) comprises a molecular weight range of from about 75 kDa to about 960 kDa. In some embodiments, the polymer (e.g., 4-arm PEG-VS) comprises a molecular weight range of from about 80 kDa to about 950 kDa. In some embodiments, the polymer (e.g., 4-arm PEG-VS) comprises a molecular weight range of from about 85 kDa to about 940 kDa. In some embodiments, the polymer (e.g., 4-arm PEG-VS) comprises a molecular weight range of from about 90 kDa to about 930 kDa. In some embodiments, the polymer (e.g., 4-arm PEG-VS) comprises a molecular weight range of from about 95 kDa to about 920 kDa. In some embodiments, the polymer (e.g., 4-arm PEG-VS) comprises a molecular weight range of from about 100 kDa to about 910 kDa. In some embodiments, the polymer (e.g., 4-arm PEG-VS) comprises a molecular weight range of from about 110 kDa to about 900 kDa. In some embodiments, the polymer (e.g., 4-arm PEG-VS) comprises a molecular weight range of from about 120 kDa to about 890 kDa. In some embodiments, the polymer (e.g., 4-arm PEG-VS) comprises a molecular weight range of from about 130 kDa to about 880 kDa. In some embodiments, the polymer (e.g., 4-arm PEG-VS) comprises a molecular weight range of from about 140 kDa to about 870 kDa. In some embodiments, the polymer (e.g., 4-arm PEG-VS) comprises a molecular weight range of from about 150 kDa to about 860 kDa. In some embodiments, the polymer (e.g., 4-arm PEG-VS) comprises a molecular weight range of from about 160 kDa to about 850 kDa. In some embodiments, the polymer (e.g., 4-arm PEG-VS) comprises a molecular weight range of from about 170 kDa to about 840 kDa. In some embodiments, the polymer (e.g., 4-arm PEG-VS) comprises a molecular weight range of from about 180 kDa to about 830 kDa. In some embodiments, the polymer (e.g., 4-arm PEG-VS) comprises a molecular weight range of from about 190 kDa to about 820 kDa. In some embodiments, the polymer (e.g., 4-arm PEG-VS) comprises a molecular weight range of from about 200 kDa to about 810 kDa. In some embodiments, the polymer (e.g., 4-arm PEG-VS) comprises a molecular weight range of from about 210 kDa to about 800 kDa. In some embodiments, the polymer (e.g., 4-arm PEG-VS) comprises a molecular weight range of from about 220 kDa to about 790 kDa. In some embodiments, the polymer (e.g., 4-arm PEG-VS) comprises a molecular weight range of from about 230 kDa to about 780 kDa. In some embodiments, the polymer (e.g., 4-arm PEG-VS) comprises a molecular weight range of from about 240 kDa to about 770 kDa. In some embodiments, the polymer (e.g., 4-arm PEG-VS) comprises a molecular weight range of from about 250 kDa to about 760 kDa. In some embodiments, the polymer (e.g., 4-arm PEG-VS) comprises a molecular weight range of from about 260 kDa to about 750 kDa. In some embodiments, the polymer (e.g., 4-arm PEG-VS) comprises a molecular weight range of from about 270 kDa to about 740 kDa. In some embodiments, the polymer (e.g., 4-arm PEG-VS) comprises a molecular weight range of from about 280 kDa to about 730 kDa. In some embodiments, the polymer (e.g., 4-arm PEG-VS) comprises a molecular weight range of from about 290 kDa to about 720 kDa. In some embodiments, the polymer (e.g., 4-arm PEG-VS) comprises a molecular weight range of from about 300 kDa to about 710 kDa. In some embodiments, the polymer (e.g., 4-arm PEG-VS) comprises a molecular weight range of from about 310 kDa to about 700 kDa. In some embodiments, the polymer (e.g., 4-arm PEG-VS) comprises a molecular weight range of from about 320 kDa to about 690 kDa. In some embodiments, the polymer (e.g., 4-arm PEG-VS) comprises a molecular weight range of from about 330 kDa to about 680 kDa. In some embodiments, the polymer (e.g., 4-arm PEG-VS) comprises a molecular weight range of from about 340 kDa to about 670 kDa. In some embodiments, the polymer (e.g., 4-arm PEG-VS) comprises a molecular weight range of from about 350 kDa to about 660 kDa. In some embodiments, the polymer (e.g., 4-arm PEG-VS) comprises a molecular weight range of from about 360 kDa to about 650 kDa. In some embodiments, the polymer (e.g., 4-arm PEG-VS) comprises a molecular weight range of from about 370 kDa to about 640 kDa. In some embodiments, the polymer (e.g., 4-arm PEG-VS) comprises a molecular weight range of from about 380 kDa to about 630 kDa. In some embodiments, the polymer (e.g., 4-arm PEG-VS) comprises a molecular weight range of from about 390 kDa to about 620 kDa. In some embodiments, the polymer (e.g., 4-arm PEG-VS) comprises a molecular weight range of from about 400 kDa to about 610 kDa. In some embodiments, the polymer (e.g., 4-arm PEG-VS) comprises a molecular weight range of from about 410 kDa to about 600 kDa. In some embodiments, the polymer (e.g., 4-arm PEG-VS) comprises a molecular weight range of from about 420 kDa to about 590 kDa. In some embodiments, the polymer (e.g., 4-arm PEG-VS) comprises a molecular weight range of from about 430 kDa to about 580 kDa. In some embodiments, the polymer (e.g., 4-arm PEG-VS) comprises a molecular weight range of from about 440 kDa to about 570 kDa. In some embodiments, the polymer (e.g., 4-arm PEG-VS) comprises a molecular weight range of from about 450 kDa to about 560 kDa. In some embodiments, the polymer (e.g., 4-arm PEG-VS) comprises a molecular weight range of from about 460 kDa to about 550 kDa. In some embodiments, the polymer (e.g., 4-arm PEG-VS) comprises a molecular weight range of from about 470 kDa to about 540 kDa. In some embodiments, the polymer (e.g., 4-arm PEG-VS) comprises a molecular weight range of from about 480 kDa to about 530 kDa. In some embodiments, the polymer (e.g., 4-arm PEG-VS) comprises a molecular weight range of from about 490 kDa to about 520 kDa. In some embodiments, the polymer (e.g., 4-arm PEG-VS) comprises a molecular weight range of from about 500 kDa to about 510 kDa.

In some embodiments, the microgel particles can be functionalized to comprise one or more functional groups. For example, a microgel particle made of hydrogel may be functionalized to comprise a functional group coupled thereto. In some embodiments, the functional group comprises a hydroxyl functional group, methyl functional group, carbonyl functional group, carboxyl functional group, amino functional group, phosphate functional group, sulfhydryl functional group, or a combination thereof. In some embodiments, the functional group comprises alkanes, alkenes, alkynes, ethers, sulfides, amines, aldehydes, ketones, imines, nitriles, or a combination thereof. In some embodiments, the functional group may be coupled to the microgel particle using a bond, linkage, interaction, or other coupling mechanism. In some embodiments, the bond is a covalent bond. In some embodiments, the bond is a non-covalent bond. In some embodiments, the bond is selected from a carbon-carbon bond, an amide bond, an imine bond, an ester bond, a thioether bond, a disulfide bond, a hydrazone bond, a hydrogen bond, and a metal ligand bond. In some embodiments, the ester bond comprises a cyclic boronate ester. In some embodiments, the linkage is selected from a carbamate linkage, an ester linkage, and a thioether linkage. In some embodiments, the coupling is selected from an oxime coupling, and a thiourea coupling. In some embodiments, the interaction is selected from an electrostatic interaction and a van der Waals interaction. In some embodiments, the functional group comprises a thiol or a derivative thereof. In some embodiments, the functional group comprises matrix metalloproteinase (MMP)-sensitive peptide. Non-limiting examples of thiol derivatives include any organosulfur compound of the form R—SH, where R represents an alkyl or other organic substituent. In some embodiments, the thiol derivatives include: methanethiol, ethanethiol, 1-propanethiol, 2-propoanethiol, allyl mercaptan, butanethiol, tert-butyl mercaptan, pentanethiols, thiophenol, dimercaptosuccinic acid, thioacetic acid, coenzyme A, glutathione, metallothionein, cysteine, 2-mercaptoethanol, dithiothreitol, dithioerythritol, 1-mercaptoindole, grapefruit mercaptan, furan-2-ylmethanethiol, 3-mercaptopropane-1, 2-diol, 3-mercapto-1-propanesulfonic acid, 1-hexadecanethiol, pentachlorobenzenethiol, or a combination thereof. In some embodiments, the functional group comprises a vinyl or a derivative thereof. In some embodiments, the functional group comprises a vinyl sulfone (VS) or a derivative thereof. Non-limiting examples of vinyl derivatives include alkenes comprising ethenes, propenes, butenes, pentenes, hexenes, heptenes, octenes, acrylate, methacrylate, acrylamide, methacrylamide, maleimide, norbornene, or a combination thereof. Non-limiting examples of VS derivatives include phenyl vinyl sulfone, methyl vinyl sulfone, ethyl vinyl sulfone, or any combination thereof. In some embodiments, the functional group comprises a thiol and VS, or derivatives of either the thiol or the VS. In some embodiments, the PEG is modified to comprise the VS or the derivative thereof to form PEG-VS. In some embodiments, the PEG is modified to comprise the thiol or the derivative thereof to form thiolated PEG (e.g., PEG-SH, PEG-dithiol). In some embodiments, the PEG-VS comprises a multi-arm PEG-VS. In some embodiments, the multi-arm PEG-VS comprises, 4-arm or 6-arm or 8-arm PEG-VS. In some embodiments, the multi-arm PEG-VS comprises star-shaped polymer, brushed polymer, branched polymer, comb polymer or dendritic polymer PEG-VS. In some embodiments, the VS comprises vinyl sulfone. In some embodiments, the PEG-VS comprises 4-arm PEG-VS.

In some embodiments, the microgel particles comprise functional groups that may be pH responsive (e.g., pH responsive microgel particles). In some embodiments, a pH responsive microgel particle may be characterized as microgel particles needing to be in the presence of a desired pH range for the annealing of the covalently stabilized scaffold to occur.

Functional groups disclosed herein may comprise a peptide. Functional groups disclosed herein may comprise an amino acid. In some embodiments, a functional group comprises a K peptide. In some embodiments, the K peptide comprises an amino acid sequence that is about 85% identical to an amino acid sequence provided in SEQ ID NO: 3. In some embodiments, the K peptides comprises an amino acid sequence comprising FKGGERCG (SEQ ID NO: 4). In some embodiments, the K peptide comprises an amino acid sequence that is about 75% identical to an amino acid sequence provided in SEQ ID NO: 3. In some embodiments, the K peptide comprises an amino acid sequence that is about 85% identical to an amino acid sequence provided in SEQ ID NO: 3. In some embodiments, the K peptide comprises an amino acid sequence that is about 95% identical to an amino acid sequence provided in SEQ ID NO: 3. In some embodiments, the K peptide comprises an amino acid sequence that is about 50%, 55%, 60%, 65%, 70%, 75%, 80%, 85%, 90%, 95%, 96%, 97%, 98%, or 99% identical to an amino acid sequence provided in SEQ ID NO: 3. In some embodiments, the K peptide comprises an amino acid sequence that is about 75% identical to an amino acid sequence provided in SEQ ID NO: 4. In some embodiments, the K peptide comprises an amino acid sequence that is about 85% identical to an amino acid sequence provided in SEQ ID NO: 4. In some embodiments, the K peptide comprises an amino acid sequence that is about 95% identical to an amino acid sequence provided in SEQ ID NO: 4. In some embodiments, the K peptide comprises an amino acid sequence that is about 50%, 55%, 60%, 65%, 70%, 75%, 80%, 85%, 90%, 95%, 96%, 97%, 98%, or 99% identical to an amino acid sequence provided in SEQ ID NO: 4. In some embodiments, the functional group comprises a Q peptide. In some embodiments, the Q peptide comprises an amino acid sequence provided in NQEQVSPLGGERCG (SEQ ID NO: 5). In some embodiments, the Q peptide comprises an amino acid sequence that is about 75% identical to an amino acid sequence provided in SEQ ID NO: 5. In some embodiments, the Q peptide comprises an amino acid sequence that is about 85% identical to an amino acid sequence provided in SEQ ID NO: 5. In some embodiments, the Q peptide comprises an amino acid sequence that is about 95% identical to an amino acid sequence provided in SEQ ID NO: 5. In some embodiments, the Q peptide comprises an amino acid sequence that is about 50%, 55%, 60%, 65%, 70%, 75%, 80%, 85%, 90%, 95%, 96%, 97%, 98%, or 99% identical to an amino acid sequence provided in SEQ ID NO: 5.

Functional groups may comprise a non-peptide polymer. Degradable functional groups may also be random sequences, Omi target sequences, Heat-Shock Protein target sequences. The functional group may comprise an amino acid having D chirality. The functional group may comprise an amino acid having L chirality. Functional groups may comprise hydrolytically degradable synthetic polymers consisting of heparin, alginate, poly(ethylene glycol), polyacrylamides, polymethacrylates, copolymers and terpolymers of polycondensates, such as polyesters, polyamides, and other polymers, such as polyurethanes). The functional group may be synthetically manufactured or naturally isolated. The functional group may comprise DNA oligonucleotides with sequences corresponding to: restriction enzyme recognition sequences, CpG motifs, Zinc finger motifs, CRISPR or Cas-9 sequences, Talon recognition sequences, or transcription factor-binding domains. The functional group may be activated on at least two ends by a reactive group, defined as a chemical group allowing the crosslinker to participate in the crosslinking reaction to form the microgel particles (intra-crosslinking within particles) or to anneal particles together to form the covalently stabilized scaffold (inter-crosslinking between particles), where these functionalities can include: cysteine amino acids, synthetic and naturally occurring thiol-containing molecules, carbene-containing groups, vinyl-containing groups, activated esters, acrylates, norbornenes, primary amines, hydrazides, phosphenes, azides, epoxy-containing groups, SANPAH containing groups, and diazirine containing groups. In some embodiments, microgel particles themselves may act as crosslinkers. In some embodiments, the functional groups may be degradable.

In some embodiments, the microgel particles may be functionalized with an acrylate, methacrylate, methacrylamide, maleimide, norbornenes, or any other vinyl derivative. For example, the hydrogel formulation may further comprise two or more acrylates, methacrylates, acrylamides, maleimides, norbornenes, or any combination thereof.

In some embodiments, the microgel particles are drug eluting, such that a therapeutic agent disclosed herein is released by the microgel particles in situ. In some embodiments, the therapeutic agent comprises a pain medication, a local anesthetic, an anti-inflammatory medication, an anti-fibrotic medication, or an antibiotic. In some embodiments, the local anesthetic is ester based. In some embodiments, the ester based local anesthetic comprises benzocaine, chloroprocaine, procaine, proparacaine, tetracaine, amylocaine, or oxybuprocaine, or any combination thereof. In some embodiments, the local anesthetic is amide based. In some embodiments, the amide based local anesthetic comprises articaine, bupivacaine, dibucaine, etidocaine, levobupivacaine, lidocaine, mepivacaine, prilocaine, ropivacaine, sameridine, tonicaine, or cinchocaine, or any combination thereof. In some embodiments, the local anesthetic is or comprises lidocaine. In some embodiments, the local anesthetic consists of lidocaine. In some embodiments, the pain medication comprises codeine, fentanyl, hydrocodone, hydromorphone, meperidine, morphine, oxycodone, or tramadol, or any combination thereof. In some embodiments, the anti-inflammatory medication is a non-steroidal anti-inflammatory drug (NSAID) or a steroid. In some embodiments, the NSAID comprises ibuprofen or naproxen. In some embodiments, the steroid comprises a corticosteroid. In some embodiments, the antibiotic comprises dicloxacillin, erythromycin, or tetracycline. In some embodiments, the anti-fibrotic medication comprises pentoxifylline.

In some embodiments herein, the microgel particles are characterized has having a degree of polydispersity. In some embodiments, the polydispersity of the microgel particles is an indicator as to the heterogeneity of the microgel particles based on size. In some embodiments, the microgel particles comprise a polydispersity of no more than 0.1 when the polydispersity (PDI) is calculated based on a standard deviation (SD) and mean size of the microgel particles (e.g., PDI=(SD/mean)\hat{ }2). In some embodiments, the polydispersity is measured using a coefficient of variant (CV), wherein the coefficient of variation is calculated based on a standard deviation (SD) and mean size of the microgel particles (e.g. CV=SD/mean). In some embodiments, a lower polydispersity, based on microgel particle size, assists in forming the covalently stabilized scaffold. In some embodiments, a lower polydispersity, based on microgel particle size, assists in achieving desired mechanical properties of the covalently stabilized scaffold. In some embodiments, a low polydispersity improves the porosity of the composition (e.g., as the size of particles becomes more polydisperse, this may lead to smaller particles inserting into the pores of the covalently stabilized scaffold).

In some embodiments, the components of the hydrogel formulation discussed above can aid in the synthesis of the microgel particles. In some embodiments, the thiol or the derivative thereof and the VS or the derivative thereof are configured to interact with each other in a reaction to synthesize the microgel particles.

In some embodiments, the reaction comprises a covalent synthesizing reaction. Non-limiting examples of a covalent bond are bonds found in a carbon-carbon, amide, ester, thioether bond, carbamate, disulfide bond, oxime, thiourea, hydrazone, and imine. In some embodiments, the reaction comprises a non-covalent synthesizing reaction. Non-limiting examples of non-covalent bonds are those found in an interaction such as, electrostatic interactions, hydrogen bonding, cation-π, π-π stacking, metal-ligand binding, and van der Waals interactions. In some embodiments, the methods comprise linking two or more microgel particles together. Non-limiting examples of linking reactions include Michael addition, amide bond coupling, “click” chemistry (e.g., Diels-Alder cycloaddition, Huisgen 1,3-dipolar cycloaddition), reductive amination, carbamate linkage, ester linkage, thioether linkage, disulfide bonding, hydrazone bonding, oxime coupling, and thiourea coupling.

In some embodiments, the reaction comprises a covalent synthesizing reaction. In some embodiments, the covalent synthesizing reaction comprises a Michael addition (e.g., thiol-ene Michael addition, aza-Michael addition, oxa-Michael addition) or a pseudo-Michael addition reaction. In some embodiments, the VS of the PEG-VS may undergo a covalent synthesizing reaction with a thiol or a derivative thereof. In some embodiments, the VS or derivative thereof and the thiol or derivative thereof is present in a ratio of VS to thiol of less than about 1.0. In some embodiments, the VS or derivative thereof and the thiol or derivative thereof is present in a ratio of VS to thiol of about 1.0, 0.9, 0.8, 0.7, 0.6, 0.5, 0.4, 0.3, 0.2, or 0.1. In some embodiments, the VS or derivative thereof and the thiol or derivative thereof is present in a ratio of thiol to VS of less than about 1.0. In some embodiments, the VS or derivative thereof and the thiol or derivative thereof is present in a ratio of thiol to VS of about 1.0, 0.9, 0.8, 0.7, 0.6, 0.5, 0.4, 0.3, 0.2, or 0.1. In some embodiments, the thiol or the derivative thereof is a Michael donor in the Michael addition or pseudo-Michael addition reaction. In some embodiments, the VS or derivative thereof is a Michael acceptor in the Michael addition or pseudo-Michael addition reaction. In some embodiments, the thiol or the derivative thereof and the VS or the derivative thereof are present in the hydrogel formulation at a molar ratio of about 1:1. In some embodiments, the thiol or the derivative thereof and the VS or the derivative thereof are present in the hydrogel formulation at a molar ratio of about 0.3:1 thiol:VS to 1:1 thiol:VS. In some embodiments, the thiol or the derivative thereof and the VS or the derivative thereof are present in the hydrogel formulation at a molar ratio of about 0.6:1 thiol:VS to 0.8:1 thiol:VS. In some embodiments, the thiol or the derivative thereof and the VS or the derivative thereof are present in the hydrogel formulation at a molar ratio of about 1:1 to about 1:2 thiol:VS. In some embodiments, the thiol or the derivative thereof and the VS or the derivative thereof are present in the hydrogel formulation at a molar ratio of about 1:1 to about 1:1.4 thiol:VS. In some embodiments, the molar ratio may be defined as the molar ratio of thiol (SH) to vinyl sulfone (VS) groups. R=[SH]/[VS]=nSH/nVS where [SH] is defined as the molar concentration of thiols, [VS] is defined as molar concentration of VS, nSH is defined as molar number of thiols and nVS is defined as molar number of VS. In some embodiments, there is an excess of either of the thiol or the derivative thereof and the VS or the derivative thereof in the hydrogel formulation such that the excess of either of the thiol or the derivative thereof or the VS or the derivative thereof participates in the annealing reaction to form the porous covalently stabilized scaffold. In some embodiments, the vinyl sulfone of the 4-arm PEG vinyl sulfone is a Michael acceptor in the Michael addition or pseudo-Michael addition reaction. In some embodiments, the thiol of the PEG-dithiol is a Michael donor in the Michael addition or pseudo-Michael addition reaction. In some embodiments, the PEG-dithiol is present in the hydrogel system in a molar concentration of at least about 0.2 millimolar (mM).

In some embodiments, the synthesis of the microgel particles may be accomplished via one or more physical linking points (e.g., looping) of the polymer(s) (e.g., PEG) that make up microgel particles. In some embodiments, physical linking may include weak physical interactions. In some embodiments, the weak physical interactions may include coordination bonding and ionic interactions. In some embodiments, the physical linking points assist the reaction in synthesizing the microgel particles. In some embodiments, microgel particle synthesis is accomplished by the reaction alone. In some embodiments, microgel particle synthesis is accomplished by physical linking alone.

### Covalently Stabilized Scaffold

Disclosed herein, in some embodiments, are microgel particles that anneal together to form a covalently stabilized scaffold. In some embodiments, the formation of the covalently stabilized scaffold is performed in situ following delivery of the microgel particles to the tissue site. In some embodiments, the hydrogel formulation comprises one or more components configured to assist in the annealing reaction to form the porous covalently stabilized scaffold.

In some embodiments, the one or more components that facilitate or induce annealing of the microgel particles in the hydrogel formulation to form the covalently stabilized scaffold comprises annealing components, annealing agents, or a combination thereof. In some embodiments, the annealing agent comprises a molecule. In some embodiments, the annealing agent comprises triethanolamine. In some embodiments, the annealing agent comprises an enzyme. In some embodiments, the enzyme comprises thrombin. In some embodiments, the annealing agent comprises a transglutaminase enzyme. A non-limiting example of a transglutaminase enzyme Factor XIII (Factor XIIIa in its active form). In some embodiments, the annealing agent comprises a radical initiator. In some embodiments, the annealing agent comprises an electron transfer agent. Examples of additional and alternative annealing agents include, by way of non-limiting example, active esters and nucleophiles, catechols that crosslink upon oxidation, and other redox sensitive molecules. In some embodiments, the annealing agents comprise homo or heterofunctional polymers containing thiols, maleimides, vinyl sulfones, methacrylates, methacrylamides, or other vinyl functionalities. In some embodiments, the annealing agent comprises cyclodextrin, cucurbituril, or calixarenes. In some embodiments, the annealing components comprise a K peptide, a Q peptide, or a combination thereof. In some embodiments, the annealing components comprise a vinyl group (e.g. vinyl sulfone, methylacrylate, acrylamide), a thiol, a maleimide, or an amine. In some embodiments, the annealing components comprise a vinyl sulfone group and a dithiol group.

In some embodiments, the microgel particles do not require an annealing agent for annealing. For example, the microgel particles may comprise other components (e.g., functional groups) that participate in a chemical crosslinking reaction to form the covalently stabilized scaffold. In some embodiments, one or more components that facilitate annealing of the microgel particles in the hydrogel formulation to form the covalently stabilized scaffold comprises a thiol derivative, a vinyl derivative, or a combination thereof. For example, microgel particles may be comprised of a polymer or copolymer that is modified to contain one or more vinyl derivatives and one or more thiol derivatives, in which either of the vinyl derivative and the thiol derivative is in excess. Such vinyl derivative and thiol derivative may undergo a chemical crosslinking reaction to anneal the microgel particles together to form the covalently stabilized scaffold.

In some embodiments, the annealing reaction to anneal the microgel particles together to form the covalently stabilized scaffold comprises a covalent synthesizing reaction. Non-limiting examples of a covalent bond are bonds found in a carbon-carbon, amide, ester, thioether bond, carbamate, disulfide bond, oxime, thiourea, hydrazone, and imine. In some embodiments, the covalent synthesizing reaction comprises a Michael addition (e.g., thiol-ene Michael addition) or a pseudo-Michael addition reaction. In some embodiments, the thiol derivative is a Michael donor in the Michael addition or pseudo-Michael addition reaction. In some embodiments, the vinyl derivative (e.g., vinyl sulfone) is a Michael acceptor in the Michael addition or pseudo-Michael addition reaction. In some embodiments, the covalent synthesizing reaction comprises an hydroxyl (oxo-Michael addition) reaction or an amine (aza-Michael addition) reaction.

In some embodiments, the functional groups and the one or more components assisting in the annealing reaction are configured to interact to perform the annealing reaction. In some embodiments, the PEG-dithiol is configured to interact with the VS of the PEG-VS in the annealing reaction to form the porous covalently stabilized scaffold. In some embodiments, the annealing reaction comprises a covalent annealing reaction. In some embodiments, the covalent annealing reaction comprises a Michael addition or pseudo-Michael addition reaction. In some embodiments, the thiol or derivative thereof of the PEG-dithiol is a Michael donor in the Michael addition or pseudo-Michael addition reaction. In some embodiments, the VS of the PEG-VS is a Michael acceptor in the Michael addition or pseudo-Michael addition reaction. In some embodiments, the PEG-dithiol is present in the hydrogel system in a molar concentration of at least about 0.2 millimolar (mM). In some embodiments, the PEG-dithiol is present in the hydrogel system in a molar concentration of at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mM. In some embodiments, the PEG-dithiol is present in the hydrogel system in a molar concentration of less than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mM. In some embodiments, the PEG-dithiol is present in the hydrogel system in a molar concentration of about 0.1 to about 2.0 mM. In some embodiments, the PEG-dithiol is present in the hydrogel system in a molar concentration of about 0.2 to about 1.9 mM. In some embodiments, the PEG-dithiol is present in the hydrogel system in a molar concentration of about 0.3 to about 1.8 mM. In some embodiments, the PEG-dithiol is present in the hydrogel system in a molar concentration of about 0.4 to about 1.7 mM. In some embodiments, the PEG-dithiol is present in the hydrogel system in a molar concentration of about 0.5 to about 1.6 mM. In some embodiments, the PEG-dithiol is present in the hydrogel system in a molar concentration of about 0.6 to about 1.5 mM. In some embodiments, the PEG-dithiol is present in the hydrogel system in a molar concentration of about 0.7 to about 1.4 mM. In some embodiments, the PEG-dithiol is present in the hydrogel system in a molar concentration of about 0.8 to about 1.3 mM. In some embodiments, the PEG-dithiol is present in the hydrogel system in a molar concentration of about 0.9 to about 1.2 mM. In some embodiments, the PEG-dithiol is present in the hydrogel system in a molar concentration of about 0.2 to about 1.0 mM. In some embodiments, the PEG-dithiol is present in the hydrogel system in a molar concentration of about 0.2 to about 0.9 mM. In some embodiments, the PEG-dithiol is present in the hydrogel system in a molar concentration of about 0.2 to about 0.7 mM. In some embodiments, the PEG-dithiol is present in the hydrogel system in a molar concentration of about 0.2 to about 0.6 mM. In some embodiments, the PEG-dithiol is present in the hydrogel system in a molar concentration of about 0.2 to about 0.5 mM.

In some embodiments, the hydrogel formulation further comprises PEG-divinyl sulfone or a derivative thereof. In some embodiments, the PEG-divinyl sulfone may be the components assisting in the annealing reaction. In some embodiments, the PEG-divinyl sulfone or derivative thereof is configured to interact with the excess thiol or derivative thereof in the annealing reaction to form the porous covalently stabilized scaffold. In some embodiments, the divinyl sulfone or derivative thereof of the PEG-divinyl sulfone is a Michael acceptor in the Michael addition or pseudo-Michael addition reaction. In some embodiments, the excess thiol is a Michael donor in the Michael addition or pseudo-Michael addition reaction.

In some embodiments, the annealing reaction to anneal the microgel particles together to form a stabilized scaffold comprises a non-covalent synthesizing reaction. Non-limiting examples of non-covalent bonds are those found in an interaction such as, electrostatic interactions, hydrogen bonding, cation-π, π-π stacking, metal-ligand binding, van der Waals interactions, and host-guest interactions such as a cyclodextrin-adamantane reaction. In some embodiments, the annealing reaction comprising a host-guest interaction may be reversible. In some embodiments, the annealing reaction to anneal the microgel particles together to form a stabilized scaffold comprises a covalent synthesizing reaction. Non-limiting examples of covalent reactions include Michael additions, amide bond coupling, “click” chemistry (e.g., Diels-Alder cycloaddition, Huisgen 1,3-dipolar cycloaddition), reductive amination, carbamate linkage, ester linkage, thioether linkage, disulfide bonding, hydrazone bonding, oxime coupling, and thiourea coupling.

In some embodiments, the molecule comprises PEG. In some embodiments, the molecule comprises PEG-dithiol. In some embodiments, the PEG-dithiol comprises a molecular weight of about 0.5 kDa to about 10 kDa. In some embodiments, the PEG-dithiol comprises a molecular weight of about 0.5, 1, 2, 3, 4, 5, 6, 7, 8, 9, 10, 11, 12, 13, 14, 15, 16, 17, 18, 19, or 20 kDa. In some embodiments, the PEG-dithiol comprises a molecular weight of greater than about 0.5, 1, 2, 3, 4, 5, 6, 7, 8, 9, 10, 11, 12, 13, 14, 15, 16, 17, 18, 19, or 20 kDa. In some embodiments, the PEG-dithiol comprises a molecular weight of less than about 0.5, 1, 2, 3, 4, 5, 6, 7, 8, 9, 10, 11, 12, 13, 14, 15, 16, 17, 18, 19, or 20 kDa. In some embodiments, the PEG-dithiol comprises a molecular weight of about 0.5 kDa to about 10.0 kDa. In some embodiments, the PEG-dithiol comprises a molecular weight of about 1.0 kDa to about 15.0 kDa. In some embodiments, the PEG-dithiol comprises a molecular weight of about 1.0 kDa to about 20.0 kDa. In some embodiments, the PEG-dithiol comprises a molecular weight of about 3.0 kDa to about 10.0 kDa. In some embodiments, the PEG-dithiol comprises a molecular weight of about 3.0 kDa to about 5.0 kDa. In some embodiments, the PEG-dithiol comprises a molecular weight of about 3.4 kDa. In some embodiments, the PEG-dithiol comprises linear PEG-dithiol, multi-arm PEG-dithiol, or a combination thereof. In some embodiments, the PEG-thiol comprises a multi-arm PEG-thiol. In some embodiments, the multi-arm PEG-thiol comprises, 4-arm or 6-arm or 8-arm PEG-thiol. In some embodiments, the multi-arm PEG-thiol comprises star-shaped polymer, brushed polymer, branched polymer, comb polymer or dendritic polymer PEG-thiol.

In some embodiments, the microgel particles are annealed to form the covalently stabilized scaffold in the presence of one or more buffers. Non-limiting examples of buffers include 4-(2-hydroxyethyl)-1-piperazineethanesulfonic acid (HEPES) buffer (pH 7.4), calcium chloride (CaCl₂)), phosphate buffered saline (PBS) (pH 6.0 to 8.0), TRIS, piperazine-N,N′-bis(2-ethanesulfonic acid) (PIPES), 2-(N-morpholino)ethanesulfonic acid buffer (MES), or a combination thereof. In some embodiments, an additional stimulus is added to catalyze the reaction, such as light or pH.

In some embodiments, the covalently stabilized scaffold is porous. In some embodiments, the covalently stabilized scaffold comprises pores having a median pore diameter comprising more than or equal to about 5 μm. In some embodiments, the pores comprise a median pore diameter of about 10 μm to about 35 μm. In some embodiments, the pores comprise a median pore diameter of greater than or equal to about 5, 10, 15, 20, 25, 30, 35, 40, 45, 50, 55, 60, 65, or 70 μm. In some embodiments, the pores comprise a median pore diameter of less than or equal to about 5, 10, 15, 20, 25, 30, 35, 40, 45, 50, 55, 60, 65, or 70 μm. In some embodiments, the pores comprise a median pore diameter of about 5 μm to about 70 μm. In some embodiments, the pores comprise a median pore diameter of about 10 μm to about 65 μm. In some embodiments, the pores comprise a median pore diameter of about 15 μm to about 60 μm. In some embodiments, the pores comprise a median pore diameter of about 20 μm to about 55 μm. In some embodiments, the pores comprise a median pore diameter of about 25 μm to about 50 μm. In some embodiments, the pores comprise a median pore diameter of about 30 μm to about 45 μm. In some embodiments, the pores comprise a median pore diameter of about 35 μm to about 40 μm. Median pore diameter may be measured by a process comprising, for a sampling of pores, (1) measuring the area of pore, (2) solving for the radius of the pore using the equation for the area of a circle (i.e., A=pi*r\hat{ }2) and (3) solving for the diameter by multiplying the radius by 2 (i.e., D=2*r).

In some embodiments, the covalently stabilized scaffold is degradable in vivo by one or more degradation pathways. In some embodiments, the one or more degradation pathways comprises oxidative degradation, enzymatic degradation, photodegradation, or hydrolytic degradation. In some embodiments, the composition of the microgel particles is fine tuned to achieve a desired degradation profile depending on the application.

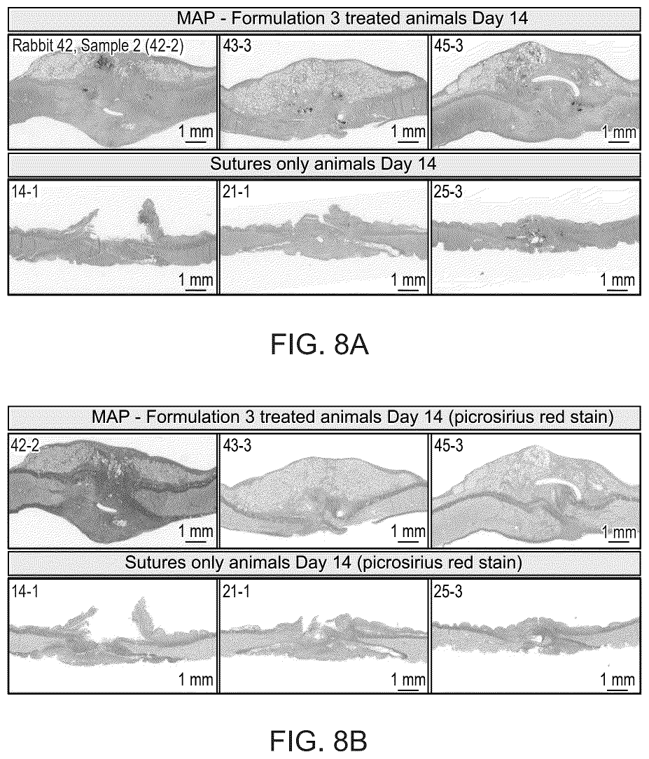

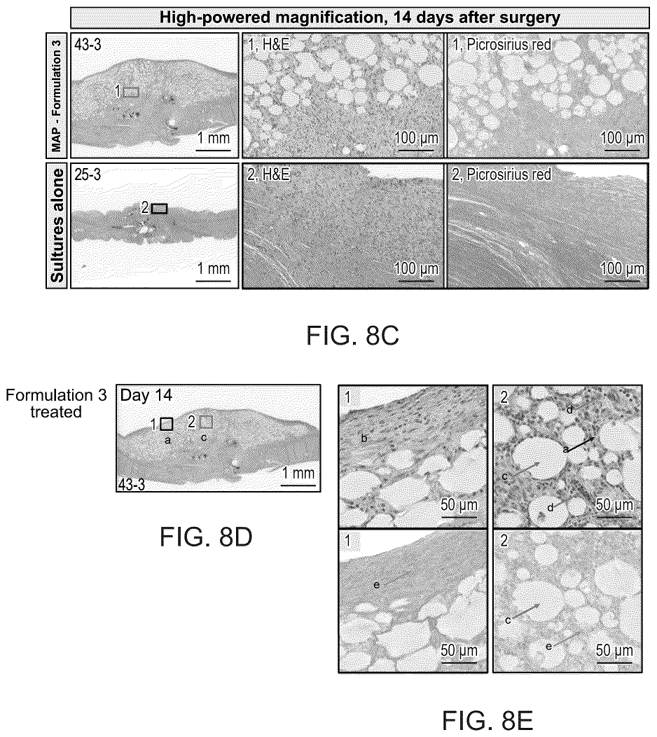

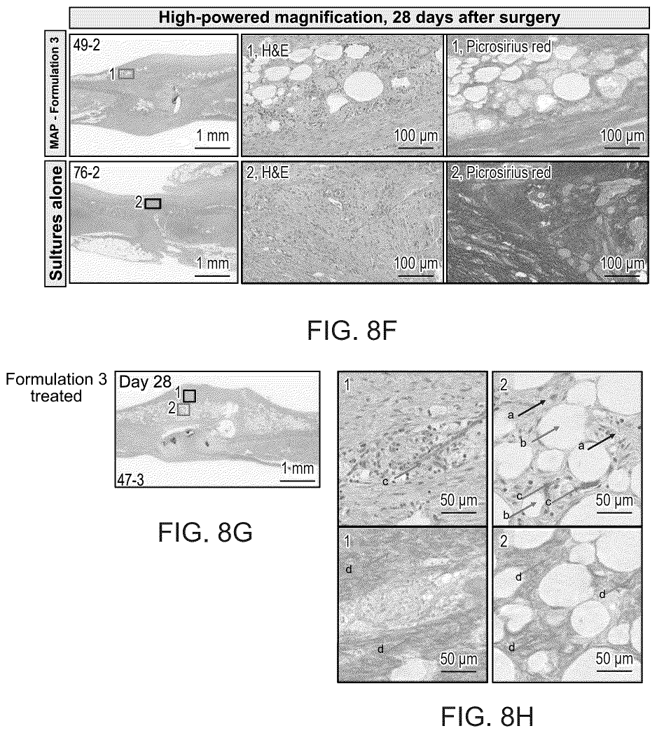

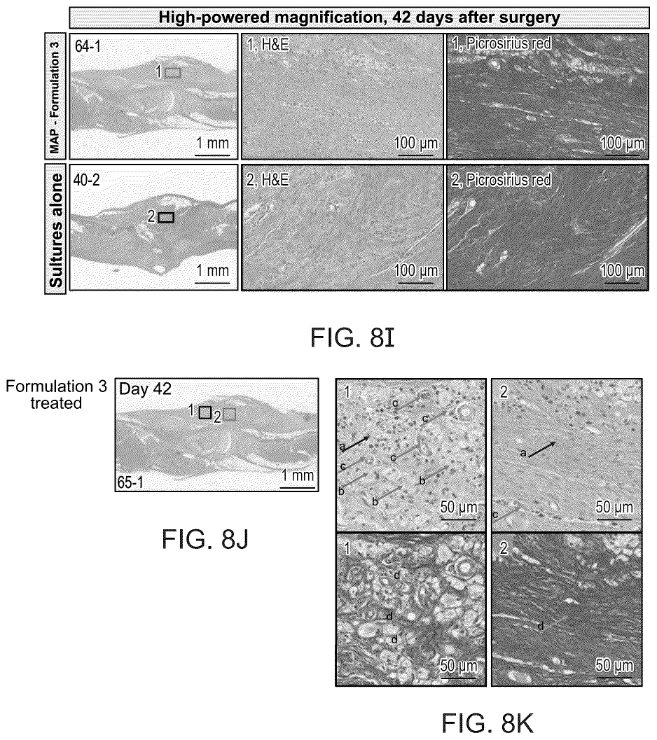

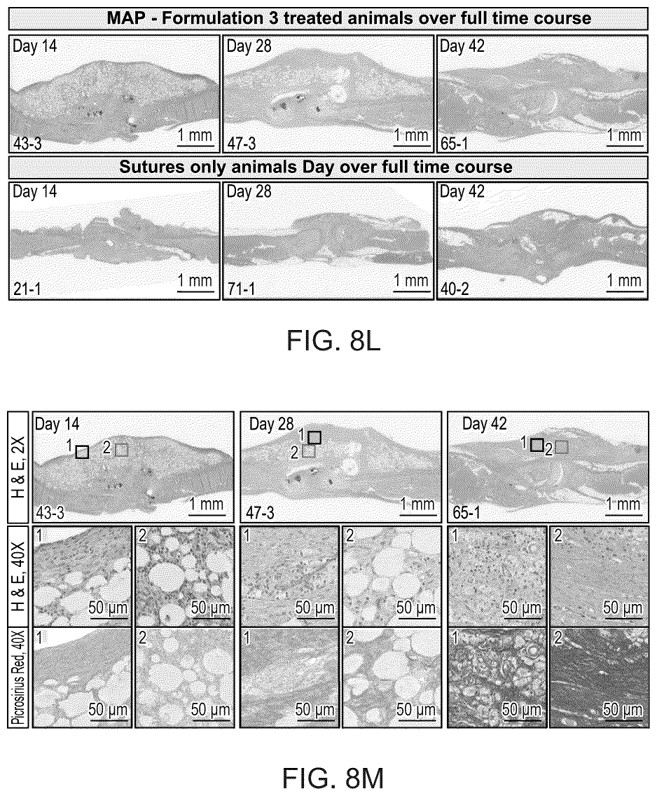

In some embodiments, the covalently stabilized scaffold is present in the tissue site for about 1 to 2 months before complete degradation. In some embodiments, the covalently stabilized scaffold is present in the tissue site for at least about 1 month before complete degradation. In some embodiments, the covalently stabilized scaffold is present in the tissue site for at least about 2 months before complete degradation. In some embodiments, the hydrogel formulations described herein allow for increased protein, cell, and tissue growth in the cell matrix. Specifically, FIGS. 8D-8K illustrates that hydrogel formulations described herein allow for perfused blood vessels within the pore space, new protein deposition within the pore space (e.g., fibrillar, non-aligned), and minimize a foreign body response, such as minimizing or completely preventing the formation of multinucleate giant cells (MNGCs).

In some embodiments, the covalently stabilized scaffold (e.g. the microgel particles after the annealing reaction) comprises an elastic compressive modulus of 1,000 Pascals (Pa) to 100,000 Pa. In some embodiments, the covalently stabilized scaffold comprises an elastic compressive modulus of at least about 1500 Pa. In some embodiments, the covalently stabilized scaffold comprises an elastic compressive modulus of at least about 6000 Pa. In some embodiments, the covalently stabilized scaffold may be annealed when the microgel particles are in a swollen and unswollen state. In some embodiments, the covalently stabilized scaffold may be annealed when the microgel particles are in a swollen state, such as with water. In some embodiments, the microgel particles are in an unswollen state when the covalently stabilized scaffold is annealed. In some embodiments, as discussed above, the elastic compressive modulus of the scaffold can be adjusted by adjusting the molecular weight, percent substitution, and molar ratios of the hydrogel polymer components.

In some embodiments, the covalently stabilized scaffold comprises an elastic compressive modulus of 5,000 Pascals (Pa) to 100,000 Pa in an unswollen state. In some embodiments, the covalently stabilized scaffold comprises an elastic compressive modulus of greater than or equal to about 5,000; 10,000; 15,000; 20,000; 25,000; 30,000; 35,000; 40,000; 45,000; 50,000; 55,000; 60,000; 65,000; 70,000; 75,000; 80,000; 85,000; 90,000; 95,000; 100,000; 105,000; 110,000; 115,000; 120,000; 125,000; 130,000; 135,000; 140,000; 145,000; or 150,000 Pa in an unswollen state. In some embodiments, the covalently stabilized scaffold comprises an elastic compressive modulus of less than or equal to about 5,000; 10,000; 15,000; 20,000; 25,000; 30,000; 35,000; 40,000; 45,000; 50,000; 55,000; 60,000; 65,000; 70,000; 75,000; 80,000; 85,000; 90,000; 95,000; 100,000; 105,000; 110,000; 115,000; 120,000; 125,000; 130,000; 135,000; 140,000; 145,000; or 150,000 Pain an unswollen state. In some embodiments, the covalently stabilized scaffold comprises an elastic compressive modulus in a range of about 5,000 Pa to about 150,000 Pa in an unswollen state. In some embodiments, the covalently stabilized scaffold comprises an elastic compressive modulus in a range of about 10,000 Pa to about 145,000 Pa in an unswollen state. In some embodiments, the covalently stabilized scaffold comprises an elastic compressive modulus in a range of about 15,000 Pa to about 140,000 Pa in an unswollen state. In some embodiments, the covalently stabilized scaffold comprises an elastic compressive modulus in a range of about 20,000 Pa to about 135,000 Pa in an unswollen state. In some embodiments, the covalently stabilized scaffold comprises an elastic compressive modulus in a range of about 25,000 Pa to about 130,000 Pa in an unswollen state. In some embodiments, the covalently stabilized scaffold comprises an elastic compressive modulus in a range of about 30,000 Pa to about 125,000 Pa in an unswollen state. In some embodiments, the covalently stabilized scaffold comprises an elastic compressive modulus in a range of about 35,000 Pa to about 120,000 Pa in an unswollen state. In some embodiments, the covalently stabilized scaffold comprises an elastic compressive modulus in a range of about 40,000 Pa to about 115,000 Pa in an unswollen state. In some embodiments, the covalently stabilized scaffold comprises an elastic compressive modulus in a range of about 45,000 Pa to about 110,000 Pa in an unswollen state. In some embodiments, the covalently stabilized scaffold comprises an elastic compressive modulus in a range of about 50,000 Pa to about 105,000 Pa in an unswollen state. In some embodiments, the covalently stabilized scaffold comprises an elastic compressive modulus in a range of about 55,000 Pa to about 100,000 Pa in an unswollen state. In some embodiments, the covalently stabilized scaffold comprises an elastic compressive modulus in a range of about 60,000 Pa to about 95,000 Pa in an unswollen state. In some embodiments, the covalently stabilized scaffold comprises an elastic compressive modulus in a range of about 65,000 Pa to about 90,000 Pa in an unswollen state. In some embodiments, the covalently stabilized scaffold comprises an elastic compressive modulus in a range of about 70,000 Pa to about 85,000 Pa in an unswollen state. In some embodiments, the covalently stabilized scaffold comprises an elastic compressive modulus in a range of about 75,000 Pa to about 80,000 Pa in an unswollen state.

In some embodiments, the covalently stabilized scaffold comprises an elastic compressive modulus of 1,000 Pascals (Pa) to 50,000 Pa in a swollen state. In some embodiments, the covalently stabilized scaffold comprises an elastic compressive modulus of greater than or equal to about 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 90,000; 95,000; 100,000; 105,000; or 110,000 Pa in a swollen state. In some embodiments, the covalently stabilized scaffold comprises an elastic compressive modulus of less than or equal to about 1,000; 2,000; 3,000; 4,000; 5,000; 6,000; 7,000; 8,000; 9,000; 10,000; 15,000; 20,000; 25,000; 30,000; 35,000; 40,000; 45,000; 50,000; 55,000; 60,000; 65,000; 70,000; 75,000; 80,000; 85,000; 90,000; 95,000; 100,000; 105,000; or 110,000 Pa in a swollen state. In some embodiments, the covalently stabilized scaffold comprises an elastic compressive modulus in a range of about 5,000 Pa to about 110,000 Pa in a swollen state. In some embodiments, the covalently stabilized scaffold comprises an elastic compressive modulus in a range of about 10,000 Pa to about 105,000 Pa in a swollen state. In some embodiments, the covalently stabilized scaffold comprises an elastic compressive modulus in a range of about 15,000 Pa to about 100,000 Pa in a swollen state. In some embodiments, the covalently stabilized scaffold comprises an elastic compressive modulus in a range of about 20,000 Pa to about 95,000 Pa in a swollen state. In some embodiments, the covalently stabilized scaffold comprises an elastic compressive modulus in a range of about 25,000 Pa to about 90,000 Pa in a swollen state. In some embodiments, the covalently stabilized scaffold comprises an elastic compressive modulus in a range of about 30,000 Pa to about 85,000 Pa in a swollen state. In some embodiments, the covalently stabilized scaffold comprises an elastic compressive modulus in a range of about 35,000 Pa to about 80,000 Pa in a swollen state. In some embodiments, the covalently stabilized scaffold comprises an elastic compressive modulus in a range of about 40,000 Pa to about 75,000 Pa in a swollen state. In some embodiments, the covalently stabilized scaffold comprises an elastic compressive modulus in a range of about 45,000 Pa to about 70,000 Pa in a swollen state. In some embodiments, the covalently stabilized scaffold comprises an elastic compressive modulus in a range of about 50,000 Pa to about 65,000 Pa in a swollen state. In some embodiments, the covalently stabilized scaffold comprises an elastic compressive modulus in a range of about 55,000 Pa to about 60,000 Pa in a swollen state.

In some embodiments, a microgel particle suspension in a swollen state is formulated for administration with a needle. In some embodiments, the microgel particle suspension in the swollen state comprises an apparent viscosity of greater than or equal to about 1, 50, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, or 10000 Pascal-second (Pa*s) when formulated for administration with a needle, and when comprising a shear rate of from about 0.1 to 10 s⁻¹. In some embodiments, the microgel particle suspension in the swollen state comprises an apparent viscosity of less than or equal to about 1, 50, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, or 10000 Pa*s when formulated for administration with a needle, and when comprising a shear rate of from about 0.1 to 10 s⁻¹. In some embodiments, the microgel particle suspension in the swollen state comprises an apparent viscosity of about 1 to about 10000 Pa*s when formulated for administration with a needle, and when comprising a shear rate of from about 0.1 to 10 s⁻¹. In some embodiments, the microgel particle suspension in the swollen state comprises an apparent viscosity of about 50 to about 9500 Pa*s when formulated for administration with a needle, and when comprising a shear rate of from about 0.1 to 10 s⁻¹. In some embodiments, the microgel particle suspension in the swollen state comprises an apparent viscosity of about 100 to about 9000 Pa*s when formulated for administration with a needle, and when comprising a shear rate of from about 0.1 to 10 s⁻¹. In some embodiments, the microgel particle suspension in the swollen state comprises an apparent viscosity of about 150 to about 8500 Pa*s when formulated for administration with a needle, and when comprising a shear rate of from about 0.1 to 10 s⁻¹. In some embodiments, the microgel particle suspension in the swollen state comprises an apparent viscosity of about 200 to about 8000 Pa*s when formulated for administration with a needle, and when comprising a shear rate of from about 0.1 to 10 s⁻¹. In some embodiments, the microgel particle suspension in the swollen state comprises an apparent viscosity of about 250 to about 7500 Pa*s when formulated for administration with a needle, and when comprising a shear rate of from about 0.1 to 10 s⁻¹. In some embodiments, the microgel particle suspension in the swollen state comprises an apparent viscosity of about 300 to about 7000 Pa*s when formulated for administration with a needle, and when comprising a shear rate of from about 0.1 to 10 s⁻¹. In some embodiments, the microgel particle suspension in the swollen state comprises an apparent viscosity of about 350 to about 6500 Pa*s when formulated for administration with a needle, and when comprising a shear rate of from about 0.1 to 10 s⁻¹. In some embodiments, the microgel particle suspension in the swollen state comprises an apparent viscosity of about 400 to about 6000 Pa*s when formulated for administration with a needle, and when comprising a shear rate of from about 0.1 to 10 s⁻¹. In some embodiments, the microgel particle suspension in the swollen state comprises an apparent viscosity of about 450 to about 5500 Pa*s when formulated for administration with a needle, and when comprising a shear rate of from about 0.1 to 10 s⁻¹. In some embodiments, the microgel particle suspension in the swollen state comprises an apparent viscosity of about 500 to about 5000 Pa*s when formulated for administration with a needle, and when comprising a shear rate of from about 0.1 to 10 s⁻¹. In some embodiments, the microgel particle suspension in the swollen state comprises an apparent viscosity of about 550 to about 4500 Pa*s when formulated for administration with a needle, and when comprising a shear rate of from about 0.1 to 10 s⁻¹. In some embodiments, the microgel particle suspension in the swollen state comprises an apparent viscosity of about 600 to about 4000 Pa*s when formulated for administration with a needle, and when comprising a shear rate of from about 0.1 to 10 s⁻¹. In some embodiments, the microgel particle suspension in the swollen state comprises an apparent viscosity of about 650 to about 3500 Pa*s when formulated for administration with a needle, and when comprising a shear rate of from about 0.1 to 10 s⁻¹. In some embodiments, the microgel particle suspension in the swollen state comprises an apparent viscosity of about 700 to about 3000 Pa*s when formulated for administration with a needle, and when comprising a shear rate of from about 0.1 to 10 s⁻¹. In some embodiments, the microgel particle suspension in the swollen state comprises an apparent viscosity of about 750 to about 2500 Pa*s when formulated for administration with a needle, and when comprising a shear rate of from about 0.1 to 10 s⁻¹. In some embodiments, the microgel particle suspension in the swollen state comprises an apparent viscosity of about 800 to about 2000 Pa*s when formulated for administration with a needle, and when comprising a shear rate of from about 0.1 to 10 s⁻¹. In some embodiments, the microgel particle suspension in the swollen state comprises an apparent viscosity of about 850 to about 1500 Pa*s when formulated for administration with a needle, and when comprising a shear rate of from about 0.1 to 10 s⁻¹. In some embodiments, the microgel particle suspension in the swollen state comprises an apparent viscosity of about 900 to about 1000 Pa*s when formulated for administration with a needle, and when comprising a shear rate of from about 0.1 to 10 s⁻¹. In some embodiments, the microgel particle suspension in the swollen state comprises an apparent viscosity of about 100 to about 1000 Pa*s when formulated for administration with a needle, and when comprising a shear rate of from about 0.1 to 10 s⁻¹.

In some embodiments, the volume fraction of the microgel particles and the elastic compressive modulus of the microgel particles can be adjusted to achieve a desired elastic compressive modulus for the covalently stabilized scaffold. Both volume fraction and elastic compressive modulus of the microgel particles effect the final elastic compressive modulus of the covalently stabilized scaffold. As shown, a higher volume fraction and higher microgel particle elastic compressive modulus may lead to a higher elastic compressive modulus of the covalently stabilized scaffold.

In some embodiments, the covalently stabilized scaffold comprises a storage modulus of 10 Pascals (Pa) to 10,000 Pa in a swollen state. In some embodiments, the covalently stabilized scaffold comprises a storage modulus of 10 Pa to 1,000 Pa in a swollen state. In some embodiments, the covalently stabilized scaffold comprises a storage modulus of greater than or equal to about 1, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10000, 10500, or 11000 Pa in a swollen state. In some embodiments, the covalently stabilized scaffold comprises a storage modulus of less than or equal to about 1, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10000, 10500, or 11000 Pa in a swollen state. In some embodiments, the covalently stabilized scaffold comprises a storage modulus in a range of about 5,000 Pa to about 110,000 Pa in a swollen state. In some embodiments, the covalently stabilized scaffold comprises a storage modulus in a range of about 10,000 Pa to about 105,000 Pa in a swollen state. In some embodiments, the covalently stabilized scaffold comprises a storage modulus in a range of about 15,000 Pa to about 100,000 Pa in a swollen state. In some embodiments, the covalently stabilized scaffold comprises a storage modulus in a range of about 20,000 Pa to about 95,000 Pa in a swollen state. In some embodiments, the covalently stabilized scaffold comprises a storage modulus in a range of about 25,000 Pa to about 90,000 Pa in a swollen state. In some embodiments, the covalently stabilized scaffold comprises a storage modulus in a range of about 30,000 Pa to about 85,000 Pa in a swollen state. In some embodiments, the covalently stabilized scaffold comprises n storage modulus in a range of about 35,000 Pa to about 80,000 Pa in a swollen state. In some embodiments, the covalently stabilized scaffold comprises a storage modulus in a range of about 40,000 Pa to about 75,000 Pa in a swollen state. In some embodiments, the covalently stabilized scaffold comprises a storage modulus in a range of about 45,000 Pa to about 70,000 Pa in a swollen state. In some embodiments, the covalently stabilized scaffold comprises a storage modulus in a range of about 50,000 Pa to about 65,000 Pa in a swollen state. In some embodiments, the covalently stabilized scaffold comprises a storage modulus in a range of about 55,000 Pa to about 60,000 Pa in a swollen state.

In some embodiments, the microgel particles comprise an elastic compressive modulus of about 1,000 Pa to about 100,000 Pa in an unswollen state. In some embodiments, the microgel particles comprise an elastic compressive modulus of about 1,000 Pa to about 50,000 Pa in an unswollen state. In some embodiments, the microgel particles comprise an elastic compressive modulus of about 1,000 Pa to about 46,000 Pa in an unswollen state. In some embodiments, the microgel particles comprise an elastic compressive modulus of about 1,000 Pa to about 75,000 Pa in an unswollen state. In some embodiments, the microgel particles comprise an elastic compressive modulus of about 1,000 Pa to about 25,000 Pa in an unswollen state. In some embodiments, the microgel particles comprise an elastic compressive modulus of greater than or equal to about 1,000, 2,000, 3,000, 4,000, 5,000, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 Pa or greater in an unswollen state. In some embodiments, the microgel particles comprise an elastic compressive modulus of less than or equal to about 1,000, 2,000, 3,000, 4,000, 5,000, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 Pa or greater in an unswollen state. In some embodiments, the microgel particles comprise an elastic compressive modulus of about 10,000 Pa to about 100,000 Pa in an unswollen state. In some embodiments, the microgel particles comprise an elastic compressive modulus of about 15,000 Pa to about 95,000 Pa in an unswollen state. In some embodiments, the microgel particles comprise an elastic compressive modulus of about 20,000 Pa to about 90,000 Pa in an unswollen state. In some embodiments, the microgel particles comprise an elastic compressive modulus of about 25,000 Pa to about 85,000 Pa in an unswollen state. In some embodiments, the microgel particles comprise an elastic compressive modulus of about 30,000 Pa to about 80,000 Pa in an unswollen state. In some embodiments, the microgel particles comprise an elastic compressive modulus of about 35,000 Pa to about 75,000 Pa in an unswollen state. In some embodiments, the microgel particles comprise an elastic compressive modulus of about 40,000 Pa to about 70,000 Pa in an unswollen state. In some embodiments, the microgel particles comprise an elastic compressive modulus of about 45,000 Pa to about 65,000 Pa in an unswollen state. In some embodiments, the microgel particles comprise an elastic compressive modulus of about 50,000 Pa to about 60,000 Pa in an unswollen state.

In some embodiments, the covalently stabilized scaffold comprises a loss modulus of about 1 Pascals (Pa) to 10,000 Pa in a swollen state. Loss modulus may be measured by undergoing a measurement of shear modulus as described above and performing an amplitude and frequency sweep of shear stress in a parallel plate system. This may enable calculation of both the storage and the loss modulus of the viscoelastic material (together the storage and loss modulus comprise the shear modulus). In some embodiments, the covalently stabilized scaffold comprises a loss modulus that is greater than or equal to about 1, 10, 20, 30, 40, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10000, 10500, or 11000 Pa in a swollen state. In some embodiments, the covalently stabilized scaffold comprises a loss modulus that is less than or equal to about 40, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10000, 10500, or 11000 Pa in a swollen state. In some embodiments, the covalently stabilized scaffold comprises a loss modulus of about 10 Pa to 11,000 Pa, 20 Pa to 10500 Pa, 30 Pa to 10000 Pa, 40 Pa to 9500 Pa, 50 Pa to 9000 Pa, 60 Pa to 8500 Pa, 70 Pa to 8000 Pa, 80 Pa to 7500 Pa, 90 Pa to 7000 Pa, 100 Pa to 6500 Pa, 150 Pa to 6000 Pa, 200 Pa to 5500 Pa, 250 Pa to 5000 Pa, 300 Pa to 4500 Pa, 350 Pa to 4000 Pa, 400 Pa to 3500 Pa, 450 Pa to 3000 Pa, 500 Pa to 2500 Pa, 550 Pa to 2000 Pa, 600 Pa to 1500 Pa, 650 Pa to 1000 Pa, 700 Pa to 950 Pa, 750 Pa to 900 Pa, or 800 Pa to 850 Pa in a swollen state.

In some embodiments, the microgel particles comprise an elastic compressive modulus of from about 500 Pa to about 50,000 Pa in a swollen state. In some embodiments, the microgel particles comprise an elastic compressive modulus of greater than about 500, 1,000, 2,000, 3,000, 4,000, 5,000, 6,000, 7,000, 8,000, 9,000, 10,000, 15,000, 20,000, 25,000, 30,000, 35,000, 40,000, 45,000, or 50,000 Pa in a swollen state. In some embodiments, the microgel particles comprise an elastic compressive modulus of less than about 500, 1,000, 2,000, 3,000, 4,000, 5,000, 6,000, 7,000, 8,000, 9,000, 10,000, 15,000, 20,000, 25,000, 30,000, 35,000, 40,000, 45,000, or 50,000 Pa in a swollen state. In some embodiments, the microgel particles comprise an elastic compressive modulus of about 500 to 50,000, 1,000 to 45,000, 2,000 to 40,000, 3,000 to 35,000, 4,000 to 30,000, 5,000 to 25,000, 6,000 to 20,000, 7,000 to 20,000, 8,000 to 15,000, or 9,000 to 10,000 Pa in a swollen state. In some embodiments, the microgel particles comprise an elastic compressive modulus of about 500 Pa to 50,000, 500 to 40,000, 500 to 30,000 500 to 20,000, or 500 to 10,000 Pa in a swollen state.

In some embodiments, the microgel particles comprise an elastic modulus of from about 5,000 to 20,000 Pa after annealing. In some embodiments, the microgel particles comprise an elastic modulus of from about 10,000 to 15,000 Pa after annealing. In some embodiments, the microgel particles comprise an elastic modulus of from about 5,000 to 15,000 Pa after annealing. In some embodiments, the microgel particles comprise an elastic modulus of greater than about 5,000; 6,000; 7,000; 8,000; 9,000; 10,000; 11,000; 12,000; 13,000; 14,000; 15,000; 16,000; 17,000; 18,000; 19,000; or 20,000 Pa after annealing. In some embodiments, the microgel particles comprise an elastic modulus of less than about 5,000; 6,000; 7,000; 8,000; 9,000; 10,000; 11,000; 12,000; 13,000; 14,000; 15,000; 16,000; 17,000; 18,000; 19,000; or 20,000 Pa after annealing.

In some embodiments, the microgel particles comprise a storage modulus of from about 10 Pa to about 5,000 Pa in a swollen state. In some embodiments, the microgel particles comprise a storage modulus of greater than about 10, 100, 500, 1,000, 1,500, 2,000, 2,500, 3,000, 3,500, 4,000, 4,500, or 5,000 Pa in a swollen state. In some embodiments, the microgel particles comprise a storage modulus of less than about 10, 100, 500, 1,000, 1,500, 2,000, 2,500, 3,000, 3,500, 4,000, 4,500, or 5,000 Pa in a swollen state. In some embodiments, the microgel particles comprise a storage modulus of about 10 to 5,000, 100 to 4,500, 500 to 4,000, or 1,000 to 3,000 Pa in a swollen state.

In some embodiments, the microgel particles comprise a storage modulus of from about 50 Pa to about 10,000 Pa in an unswollen state. In some embodiments, the microgel particles comprise a storage modulus of greater than about 50, 100, 500, 1,000, 2,000, 3,000, 4,000, 5,000, 6,000, 7,000, 8,000, 9,000, or 10,000 Pa in an unswollen state. In some embodiments, the microgel particles comprise a storage modulus of less than about 50, 100, 500, 1,000, 2,000, 3,000, 4,000, 5,000, 6,000, 7,000, 8,000, 9,000, or 10,000 Pain an unswollen state. In some embodiments, the microgel particles comprise a storage modulus of about 50 to 10,000, 100 to 9,000, 500 to 8,000, 1,000 to 7,000, 2,000 to 6,000, or 3,000 to 5,000 Pa in an unswollen state.

In some embodiments, the microgel particles comprise a loss modulus of from about 0.1 Pa to about 2,000 Pa in a swollen state. In some embodiments, the microgel particles comprise a loss modulus of greater than about 0.1, 0.5, 1, 50, 100, 500, 1,000, 1,500, or 2,000 Pa in a swollen state. In some embodiments, the microgel particles comprise a loss modulus of less than about 0.1, 0.5, 1, 50, 100, 500, 1,000, 1,500, or 2,000 Pa in a swollen state. In some embodiments, the microgel particles comprise a loss modulus of about 0.1 to 2,000, 0.5 to 1,500, 1 to 1,000, or 50 to 500 Pa in a swollen state.

In some embodiments, the microgel particles comprise a loss modulus of from about 1 Pa to about 5,000 Pa in an unswollen state. In some embodiments, the microgel particles comprise a loss modulus of greater than about 1, 100, 500, 1,000, 1,500, 2,000, 2,500, 3,000, 3,500, 4,000, 4,500, or 5,000 Pa in an unswollen state. In some embodiments, the microgel particles comprise a loss modulus of less than about 1, 100, 500, 1,000, 1,500, 2,000, 2,500, 3,000, 3,500, 4,000, 4,500, or 5,000 Pa in an unswollen state. In some embodiments, the microgel particles comprise a loss modulus of about 1 to 5,000, 100 to 4,500, 500 to 4,000, or 1,000 to 3,000 Pa in an unswollen state.

In some embodiments, a microgel particle suspension comprises an elastic compressive modulus of from about 100 Pa to about 20,000 Pa in a swollen state. In some embodiments, a microgel particle suspension comprises an elastic compressive modulus of greater than about 100, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, or 20,000 Pa in a swollen state. In some embodiments, a microgel particle suspension comprises an elastic compressive modulus of less than about 100, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, or 20,000 Pa in a swollen state. In some embodiments, a microgel particle suspension comprises an elastic compressive modulus of about 100 to 20,000, 500 to 19,000, 1,000 to 18,000, 2,000 to 17,000, 3,000 to 16,000, 4,000 to 15,000, 5,000 to 14,000, 6,000 to 13,000, 7,000 to 12,000, 8,000 to 11,000, or 9,000 to 10,000 Pa in a swollen state.

In some embodiments, a microgel particle suspension comprises an elastic compressive modulus of from about 500 Pa to about 50,000 Pa in an unswollen state. In some embodiments, a microgel particle suspension comprises an elastic compressive modulus of greater than about 500, 1,000, 2,000, 3,000, 4,000, 5,000, 6,000, 7,000, 8,000, 9,000, 10,000, 15,000, 20,000, 25,000, 30,000, 35,000, 40,000, 45,000, or 50,000 Pa in an unswollen state. In some embodiments, a microgel particle suspension comprises an elastic compressive modulus of less than about 500, 1,000, 2,000, 3,000, 4,000, 5,000, 6,000, 7,000, 8,000, 9,000, 10,000, 15,000, 20,000, 25,000, 30,000, 35,000, 40,000, 45,000, or 50,000 Pa in an unswollen state. In some embodiments, a microgel particle suspension comprises an elastic compressive modulus of about 500 to 50,000, 1,000 to 45,000, 2,000 to 40,000, 3,000 to 35,000, 4,000 to 30,000, 5,000 to 25,000, 6,000 to 20,000, 7,000 to 20,000, 8,000 to 15,000, or 9,000 to 10,000 Pa in an unswollen state. In some embodiments, a microgel particle suspension comprises an elastic compressive modulus of about 500 Pa to 50,000, 500 to 40,000, 500 to 30,000 500 to 20,000, or 500 to 10,000 Pa in an unswollen state.

In some embodiments, a microgel particle suspension comprises a storage modulus of from about 1 to about 10,000 Pa in a swollen state. In some embodiments, a microgel particle suspension comprises a storage modulus of greater than about 1, 2, 3, 4, 5, 6, 7, 8, 9, 10, 100, 500, 1,000, 2,000, 3,000, 4,000, 5,000, 6,000, 7,000, 8,000, 9,000 or 10,000 Pa in a swollen state. In some embodiments, a microgel particle suspension comprises a storage modulus of less than about 1, 2, 3, 4, 5, 6, 7, 8, 9, 10, 100, 500, 1,000, 2,000, 3,000, 4,000, 5,000, 6,000, 7,000, 8,000, 9,000 or 10,000 Pa in a swollen state. In some embodiments, a microgel particle suspension comprises a storage modulus of about 1 to 10,000, 10 to 9,000, 100 to 8,000, 500 to 7,000, 1,000 to 6,000, or 2,000 to 5,000 Pa in a swollen state.

In some embodiments, a microgel particle suspension comprises a loss modulus of from about 1 to about 10,000 Pa in a swollen state. In some embodiments, a microgel particle suspension comprises a loss modulus of greater than about 1, 100, 500, 1,000, 2,000, 3,000, 4,000, 5,000, 6,000, 7,000, 8,000, 9,000 or 10,000 Pa in a swollen state. In some embodiments, a microgel particle suspension comprises a loss modulus of less than about 1, 100, 500, 1,000, 2,000, 3,000, 4,000, 5,000, 6,000, 7,000, 8,000, 9,000 or 10,000 Pa in a swollen state. In some embodiments, a microgel particle suspension comprises a loss modulus of about 1 to 10,000, 100 to 9,000, 500 to 8,000, 1,000 to 7,000, 2,000 to 6,000, or 3,000 to 5,000 Pa in a swollen state.

In some embodiments, the thiol or the derivative thereof and the vinyl sulfone or the derivative thereof are present in the hydrogel formulation at a molar ratio of the thiols to the VS (thiol:VS) of about 0.3 to about 0.95 to achieve a desired elastic compressive modulus of about 500 Pa to about 50,000 Pa (e.g., when the hydrogel formulation is formulated for administration with a needle). In some embodiments, the microgel particles are present in a suspension comprising the microgel particles and water and wherein a 50% to 100% volume fraction of the suspension comprises the microgel particles to achieve a desired elastic compressive modulus (e.g., when the hydrogel formulation is formulated for administration with a needle).

In some embodiments, the covalently stabilized scaffold comprises an apparent viscosity of about 1 to about 10,000 pascal-second (Pa s) in the shear rate range of 0.1 to 10 s⁻¹. In some embodiments, the volume fraction of the microgel particles and the elastic compressive modulus of the microgel particles can be adjusted to achieve a desired viscosity.

In some embodiments, the covalently stabilized scaffold comprises a pH of 5.0 to 9.0. In some embodiments, the covalently stabilized scaffold comprises a pH of about 7 to about 9. In some embodiments, the pH is about 8.0 In some embodiments, the covalently stabilized scaffold comprises a pH of greater than or equal to about 4.0, 4.5, 5.0, 5.5, 6.0, 6.5, 7.0, 7.5, 8.0, 8.5, 9.0, 9.5, or 10.0. In some embodiments, the covalently stabilized scaffold comprises a pH of less than or equal to about 4.0, 4.5, 5.0, 5.5, 6.0, 6.5, 7.0, 7.5, 8.0, 8.5, 9.0, 9.5, or 10.0. In some embodiments, the covalently stabilized scaffold comprises a pH of about 4.0 to about 10.0. In some embodiments, the covalently stabilized scaffold comprises a pH of about 4.5 to about 9.5. In some embodiments, the covalently stabilized scaffold comprises a pH of about 5.0 to about 9.0. In some embodiments, the covalently stabilized scaffold comprises a pH of about 5.5 to about 8.5. In some embodiments, the covalently stabilized scaffold comprises a pH of about 6.0 to about 8.0. In some embodiments, the covalently stabilized scaffold comprises a pH of about 6.5 to about 7.5. In some embodiments, the covalently stabilized scaffold comprises a pH of about 7.0 to about 7.5. In some embodiments, the covalently stabilized scaffold comprises a pH of about 7.0 to about 9.0. In some embodiments, the covalently stabilized scaffold comprises a pH of about 7.5 to about 8.5. In some embodiments, the covalently stabilized scaffold comprises a pH of about 8.0 to about 8.5.

In some embodiments, the microgel particles comprise a pH of 5.0 to 9.0. In some embodiments, the microgel particles comprise a pH of 6.5 to 7.5. In some embodiments, the microgel particles comprise a pH of greater than or equal to about 4.0, 4.5, 5.0, 5.5, 6.0, 6.5, 7.0, 7.5, 8.0, 8.5, 9.0, 9.5, or 10.0. In some embodiments, the microgel particles comprise a pH of less than or equal to about 4.0, 4.5, 5.0, 5.5, 6.0, 6.5, 7.0, 7.5, 8.0, 8.5, 9.0, 9.5, or 10.0. In some embodiments, the microgel particles comprise a pH of about 4.0 to about 10.0. In some embodiments, the microgel particles comprise a pH of about 4.5 to about 9.5. In some embodiments, the microgel particles comprise a pH of about 5.0 to about 9.0. In some embodiments, the microgel particles comprise a pH of about 5.5 to about 8.5. In some embodiments, the microgel particles comprise a pH of about 6.0 to about 8.0. In some embodiments, the microgel particles comprise a pH of about 6.5 to about 7.5. In some embodiments, the microgel particles comprise a pH of about 7.0 to about 7.5.

### Hydrogel Formulations

Disclosed herein, in some embodiments, are hydrogel formulations comprising the microgel particles of the present disclosure and an additional active agent (e.g., therapeutic agent), reagent or solvent.

In some embodiments, the hydrogel formulation comprises microgel particles and a therapeutic agent. In some embodiments, the therapeutic agent is not released from the microgel particles. In some embodiments, the therapeutic agent is released from the microgel particles (e.g., drug-eluting microgel particle). In some embodiments, the active agent is a therapeutic agent. In some embodiments, the therapeutic agent comprises an antibiotic.

In some embodiments, the hydrogel formulation is capable of withstanding sterilization. Sterilization can be carried out by steam sterilization, filtration, microfiltration, e-beam, gamma radiation, ethylene oxide (ETO), light, supercritical carbon dioxide, vaporized hydrogen peroxide or any combination thereof. In some embodiments, certain components of the hydrogel formulation may be steam sterilized (e.g., autoclaved) without degradation of physical properties, such as the microgel particles. However, when the hydrogel formulation contains an additional component, such as the therapeutic agent, the component of the hydrogel formulation containing the therapeutic agent can be sterilized by means other than heat treatment, such as for example using filtration sterilization.

In some embodiments, sterilization of the hydrogel formulation is by autoclave. Autoclaving can be accomplished by applying a mixture of heat, pressure, and moisture to a formulation in need of sterilization. Many different sterilization temperatures, pressures and cycle times can be used. As an example, in some embodiments, the filled syringes may be sterilized at a temperature of at least about 120° C. to about 130° C. or greater. In some embodiments, the filled syringes may be sterilized at a temperature of at least about 120° C. to about 130° C. or greater. Moisture may or may not be utilized. In some embodiments, pressure is applied depending on the temperature used in the sterilization process. In some embodiments, the sterilization cycle may be at least about 1 minute to about 20 minutes or more. In some embodiments, the sterilization cycle may be at least about 1 minute to about 30 minutes or more. In some embodiments, the sterilization cycle may be at least about 15 minutes to about 30 minutes or more.

In some embodiments, the method of sterilization incorporates the use of a gaseous species which is known to kill or eliminate transmissible agents. In some embodiments, ethylene oxide is used as the sterilization gas and can sterilize medical devices, products, or any of the delivery devices disclosed herein.

In some embodiments, the method of sterilization incorporates the use of an irradiation source to kill or eliminate transmissible agents. A beam of irradiation is targeted at the delivery device (e.g., syringe) containing the hydrogel formulation, and the wavelength of energy kills or eliminates the unwanted transmissible agents. As a non-limiting example, energy useful includes, but is not limited to ultraviolet (UV) light, electron (e-beam) irradiation, gamma irradiation, visible light, microwaves, or any other wavelength or band of wavelengths which kills or eliminates the unwanted transmissible agents, preferably without substantially altering of degrading the hydrogel formulation.

In some embodiments, the hydrogel formulation comprises a reagent, such as an annealing agent that facilitates the annealing reaction of the hydrogel formulation to form a covalently stabilized scaffold. In some embodiments, the annealing agent comprises PEG-dithiol. In some embodiments, the annealing agent comprises a photoinitiator. By way of non-limiting example, the photoinitiator may be Eosin Y. In some embodiments, the annealing agent comprises triethanolamine. In some embodiments, the annealing agent comprises an enzyme. In some embodiments, the enzyme comprises thrombin. In some embodiments, the annealing agent comprises a transglutaminase enzyme. A non-limiting example of a transglutaminase enzyme Factor XIII (Factor XIIIa in its active form). In some embodiments, the annealing agent is endogenous to the subject or the tissue. For example, an enzyme (e.g., Factor XIII or Factor XIIIa) may be present naturally in the tissue, which can participate in an annealing reaction of the hydrogel formulation in situ provided that the appropriate annealing components are present in the hydrogel formulation. In some embodiments, the annealing agent is autologous to the subject or tissue. For example, endogenous Factor XIII may be obtained from the subject, and delivered to the subject to participate in the annealing reaction of the hydrogel formulation in situ provided that the appropriate annealing components are present. In some embodiments, the Factor XIII is modified prior to delivery to the tissue or subject. In some embodiments, the annealing agent comprises a co-initiator. In some embodiments, the annealing agent comprises an electron transfer agent. Examples of additional and alternative annealing agents include, by way of non-limiting example, include active esters and nucleophiles, catechols that crosslink upon oxidation, and other redox sensitive molecules. In some embodiments, the annealing components comprise a K peptide, a Q peptide, or a combination thereof. In some embodiments, the reagent comprises a stabilization agent, a sterilization agent, or a heat protectant. Non-limiting examples of stabilization agents include reagents, salts, and additives. Non-limiting examples of sterilization agents include reagents, salts, and additives. Non-limiting examples of heat protectants include antioxidants, glycerine, and PEG. In some embodiments, the hydrogel formulation is protected during sterilization by freezing the hydrogel prior and/or during the terminal sterilization (e.g. irradiation). In some embodiments, the hydrogel formulation is protected during sterilization by placing the material under a sealed inert atmosphere or under sous-vide ampule.

In some embodiments, the hydrogel formulation comprises a solvent, such as one or more buffers, water, or a combination thereof. In some embodiments, the microgel particles are present in a suspension comprising water. In some embodiments, the hydrogel formulation comprises a buffer. In some embodiments, the buffer comprises: a phosphate buffer, a 4-(2-hydroxyethyl)-1-piperazineethanesulfonic acid (HEPES) buffer, a phosphate buffer, or an acetate buffer, a citrate buffer, a borate buffer, or any combination thereof. In some embodiments, the buffer adjusts the pH of the hydrogel formulation to a desired pH. The pH of the disclosed hydrogel formulations can be about 5.0 to about 8.0, or about 6.5 to about 7.5. In certain embodiments, the pH of the formulation is about 7.0 to about 7.4 or about 7.1 to about 7.3. In some embodiments, the hydrogel formulation comprises a suspension of the microgel particles in an aqueous solvent, including the buffer. In some embodiments, the buffer may be a buffering agent. In some embodiments, a 50% to 100% volume fraction of the suspension comprises the microgel particles. In some embodiments, at least a 30%, 40%, 50%, 60%, 70%, 80%, 90%, or 100% volume fraction of the suspension comprises the microgel particles. In some embodiments, the volume fraction of the microgel particles is greater than or equal to about 50% when the hydrogel is formulated for administration with a needle. In some embodiments, the volume fraction of the microgel particles is greater than or equal to about 50%, 60%, 70%, 80%, or 90% when the hydrogel is formulated for administration with a needle.

In some embodiments, the hydrogel formulation is colorless. In some embodiments, the hydrogel formulation is substantially optically clear. In some embodiments, the polydispersity of the hydrogel formulation is no more than 0.1. In some embodiments, the coefficient of variance of the hydrogel formulation is no more than about 62.5%, 60%, 57.5%, 55%, 52.5%, 50%, 47.5%, 45%, 42.5%, 40%, 37.5%, 35%, 32.5%, 30%, 27.5%, 25%, 22.5%, 20%, 17.5%, 15%, 12.5%, or 10%. In some embodiments, the coefficient of variance of the hydrogel formulation is no more than about 62.5%. In some embodiments, the coefficient of variance of the hydrogel formulation is no more than about 30%. In some embodiments, the hydrogel formulation is odorless.

In some embodiments, the hydrogel formulation is formulated for administration to a subject. In some embodiments, the administration is subdermal administration, dermal administration, intradermal administration, or subcutaneous administration. In some embodiments, administration minimizes a foreign body response in the subject. In some embodiments, the hydrogel formulation is formulated for administration by a needle. In some embodiments, the hydrogel formulation is formulated for administration by a microneedle or microneedle patch. In some embodiments, the hydrogel formulation is formulated for administration by a needle that has a gauge that is about 26-, 27-, 28-, 29-, or 30-gauge. For example, a hydrogel formulation formulated for a 27-gauge syringe may have an apparent viscosity of about 1 to 1000 Pascal*seconds when measured at shear rates between 0.1 and 10 s⁻¹. While a hydrogel formulation formulated for a 30-gauge syringe may have an apparent viscosity of about 1 to 500 Pascal*seconds when measured at shear rates between 0.1 and 10 s⁻¹. Thus, the hydrogel formulation properties may be fine-tuned depending on the mode of administration.

In some embodiments, a dose of the hydrogel formulation comprises a volume of about 0.01 mL to about 20 mL. In some embodiments, the volume comprises about 0.75 milliliter (mL) to about 1.0 mL. In some embodiments, the volume comprises about 0.5 mL to about 3.0 mL. In some embodiments, the volume comprises 0.75 mL to about 2.75 mL, 1.0 mL to about 2.25 mL, 1.25 mL to about 2.0 mL, 1.0 mL to about 1.75 mL, or 1.25 mL to 1.50 mL. In some embodiments, the dose comprises at greater than or equal to about 0.01 mL, 0.10 mL, 0.20 mL, 0.30 mL, 0.40 mL, 0.50 mL, 0.75 mL, 1.0 mL, 1.25 mL, 1.5 mL, 1.75 mL, 2.0 mL, 2.25 mL, 2.5 mL, 2.75 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, or 20 mL.

In some embodiments, the hydrogel formulation is aseptically manufactured. In some embodiments, the hydrogel formulation is sterile. In some embodiments, the hydrogel formulation is formulated for sterilization by steam sterilization, filtration, microfiltration, e-beam, gamma radiation, ethylene oxide (ETO), light, supercritical carbon dioxide, vaporized hydrogen peroxide or any combination thereof. In some embodiments, the hydrogel formulation comprises at least two separate containers, each container suitable for sterilization by different methods. In some embodiments, the microgel particles are lyophilized. In some embodiments, the lyophilized microgel particles are stored in a first container that is capable of withstanding steam sterilization, which is separate from a second container comprising components of the hydrogel formulation that may be degradable by steam sterilization, such as the therapeutic agent. In another embodiment, the hydrogel formulation is stored in a single container capable of being sterilized together. In some embodiments, the system also comprises a reconstitution medium to reconstitute the lyophilized hydrogel formulation. In some embodiments, the reconstitution medium comprises a physiologically isotonic buffer such as phosphate buffered saline. In some embodiments, the reconstitution medium has a pH higher than the physiological pH. In some embodiments, the reconstitution medium has a pH lower than the physiological pH. In some embodiments, the reconstitution medium comprises a buffer with a varying buffer capacity.

### Delivery Devices

Disclosed herein, in some embodiments, are delivery devices configured to deliver the hydrogel formulation to a subject. A non-limiting example of a delivery device is a needle, or a microneedle (e.g., microneedle patch). In some embodiments, the delivery device comprises a body and an applicator in fluidic communication with the body. In some embodiments, the body is elongated (e.g., a barrel). In some embodiments, the body of the delivery device comprises an inner chamber that contains the hydrogel formulation. In some embodiments, the delivery device comprises a pump or a plunger configured to apply pressure to the hydrogel formulation contained in the body under conditions that the hydrogel formulation flows through and out of the applicator via an outlet of the applicator. In some embodiments, the body of the delivery device comprises a first chamber for the microgel particles and a second chamber for the annealing agents and/or components. In some embodiments, the delivery device mixes the components of the first chamber and the second chamber. In some embodiments, the syringe is prefilled with the hydrogel formulation. In some embodiments the syringe is sterile. In some embodiments, the syringe is packaged separately from the hydrogel formulation and both the syringe, and the hydrogel formulation are sterile.

In some embodiments, the syringe comprises a needle. In some embodiments, the needly comprises a blunt needle. In some embodiments, the needle has a gauge comprising from about 10 gauge to about 20 gauge. In some embodiments, the needle has a gauge comprising about 18 gauge.

### Methods

Disclosed herein, in some embodiments, are methods of preparing or using the hydrogel formulations disclosed herein. In some embodiments, the methods disclosed herein comprise delivering the hydrogel formulations disclosed herein to a subject. In some embodiments, the delivering comprising administering the hydrogel formulations to the subject. In some embodiments, administration comprises subdermal, dermal, intradermal, or subcutaneous administration of the hydrogel formulation to a tissue site of the subject. In some embodiments, administration comprises administering the hydrogel formulation at the site of an incision or suture. In some embodiments, administering the hydrogel formulation to a tissue site of the subject is effective to treat the tissue at or surrounding the tissue site, such as for example, improving the strength of an incision or improving the healing at a site of a suture line. In some embodiments, methods of delivering or administering the hydrogel formulation disclosed herein is performed while minimizing a foreign body response elicited by the subject in response to the delivering or the administering. Also provided are methods for purifying the microgel particles of the hydrogel formulations disclosed herein, such as for example, in a water-in-oil emulsion. In some embodiment, the methods comprise lyophilizing the microgel particles to that they may be stored and/or distributed over long periods of time prior to being reconstituted and delivered at the point of need to the subject.

### Methods of Delivery

Disclosed herein, in some embodiments, are methods of delivering the hydrogel formulations provided herein to a subject. In some embodiments, the hydrogel formulation is delivered to a tissue site of a subject. In some embodiments, the tissue site is a site of a suture line. In some embodiments, the hydrogel formulation is delivered to a site of a suture line. In some embodiments, the method comprises delivering to the tissue site the hydrogel formulation under conditions sufficient for the microgel particles to anneal to one another to form a porous covalently stabilized scaffold. In some embodiments, the formation of the porous covalently stabilized scaffold happens in vivo, in situ, or both in vivo and in situ. In some embodiments, the porous covalently stabilized scaffold forms under conditions sufficient to allow cells to grow within the pores of the porous covalently stabilized scaffold to produce a cell matrix. In some embodiments, the cell matrix that forms persists in the subject after complete degradation of the porous covalently stabilized scaffold, thereby permanently filling at least a part of the tissue site of the subject while minimizing a foreign body response in the subject. The methods of delivery disclosed herein may be subdermal, dermal, intradermal, or subcutaneous. In some embodiments, the methods of delivery comprise injection, such as for example, using a delivery device (e.g., syringe) disclosed herein.

In some embodiments, the suture line was an incision. In some embodiments, the hydrogel formulation is delivered on top of the suture line after the suture line is sutured. In some embodiments, the hydrogel formulation is delivered into the suture line while the suture line is sutured. In some embodiments, the hydrogel formulation is delivered on top of the suture line after the suture line is sutured and into the suture line while the suture line is sutured. In some embodiments, the hydrogel begins to anneal on top of the suture line for at least 1, 2, 3, 4, 15, 6, 7, 8, 9, 10, 11, 12, 13, 14, 15, 16, 17, 18, 19, or 20 minutes before the dermis and subcutis of the subject is sutured. In some embodiments, the suture line was an abdominal incision below the dermis and the subcutis of the subject. In some embodiments, the hydrogel begins to anneal on top of the incision for at least about 10 minutes before the dermis and subcutis is closed. In some embodiments, the hydrogel begins to anneal on top of the incision for at least about 1 minute before the dermis and subcutis is closed. In some embodiments, the hydrogel begins to anneal on top of the incision for at least about, 2, 3, 4, 5, 6, 7, 8, 9, 10, 11, 12, 13, 14, 15, 16, 17, 18, 19, or 20 minutes before the dermis and subcutis is closed. In some embodiments, the hydrogel annealing strengthens the suture line at the suture line site.

In some embodiments, the methods further comprise strengthening the suture line at the site of a suture line by the cell matrix formed over the site of a suture line. In some embodiments, the strengthening the suture line is characterized by increasing a mechanical tensile strength of the suture line as compared to a reference suture line at an otherwise identical site of a suture line that was closed without the delivery of the hydrogel formulation. In some embodiments, the increasing the mechanical tensile strength of the suture line is characterized by increasing the yield stress of the suture line. The yield stress may be calculated from a stress versus strain curve measured using a tensile test (e.g., on an Instron). A non-limiting example of this is depicted in FIG. 6A.

In some embodiments, the yield stress is calculated from a stress versus strain curve measured using a tensile test (e.g., on an Instron 3342). In some embodiments, the suture line comprises a yield stress of at least about 4.0 Newtons per millimeter squared (N/mm²) measured after closure. In some embodiments, the suture line comprises a yield stress at least about 1.0 N/mm², about 1.5 N/mm², about 2.0 N/mm², about 2.5 N/mm², about 3.0 N/mm², about 3.5 N/mm², about 4.0 N/mm², about 4.5 N/mm², about 5.0 N/mm², about 5.5 N/mm², about 6.0 N/mm², about 6.5 N/mm², about 7.0 N/mm², about 7.5 N/mm², about 8.0 N/mm², about 8.5 N/mm², about 9.0 N/mm², about 9.5 N/mm², about 10.0 N/mm², about 15.0 N/mm², about 20.0 N/mm², about 25.0 N/mm², about 30.0 N/mm², about 35.0 N/mm², about 40.0 N/mm², about 45.0 N/mm², about 50.0 N/mm², about 55.0 N/mm² about 60.0 N/mm², about 65.0 N/mm² about 70.0 N/mm², about 75.0 N/mm² about 80.0 N/mm², about 85.0 N/mm² about 90.0 N/mm², about 95.0 N/mm², or about 100.0 N/mm². In some embodiments, the suture line comprises a yield stress of no more than about 1.0 N/mm², about 1.5 N/mm², about 2.0 N/mm², about 2.5 N/mm², about 3.0 N/mm², about 3.5 N/mm², about 4.0 N/mm², about 4.5 N/mm², about 5.0 N/mm², about 5.5 N/mm², about 6.0 N/mm², about 6.5 N/mm², about 7.0 N/mm², about 7.5 N/mm², about 8.0 N/mm², about 8.5 N/mm², about 9.0 N/mm², about 9.5 N/mm², about 10.0 N/mm², about 15.0 N/mm², about 20.0 N/mm², about 25.0 N/mm², about 30.0 N/mm², about 35.0 N/mm², about 40.0 N/mm², about 45.0 N/mm², about 50.0 N/mm², about 55.0 N/mm² about 60.0 N/mm², about 65.0 N/mm² about 70.0 N/mm², about 75.0 N/mm² about 80.0 N/mm², about 85.0 N/mm² about 90.0 N/mm², about 95.0 N/mm², or about 100.0 N/mm². In some embodiments, the yield stress is measured at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days after closure. In some embodiments, the yield stress is measured at least about 42 days after closure.

In some embodiments, the suture line comprises a yield stress of at least about 3.0 N/mm² to about 6.0 N/mm² after closure. In some embodiments, the suture line comprises a yield stress of at least about 4.0 N/mm² to about 5.0 N/mm² after closure. In some embodiments, the suture line comprises a yield stress of at least about 3.0 N/mm² to about 4.0 N/mm² after closure. In some embodiments, the suture line comprises a yield stress of greater than about 3.0 N/mm², about 3.5 N/mm², about 4.0 N/mm², about 4.5 N/mm², about 5.0 N/mm², about 5.5 N/mm², about 6.0 N/mm², about 6.5 N/mm², about 7.0 N/mm², about 7.5 N/mm², about 8.0 N/mm², about 8.5 N/mm², about 9.0 N/mm², about 9.5 N/mm², about 10.0 N/mm², about 15.0 N/mm², about 20.0 N/mm², about 25.0 N/mm², about 30.0 N/mm², about 35.0 N/mm², about 40.0 N/mm², about 45.0 N/mm², about 50.0 N/mm², about 55.0 N/mm² about 60.0 N/mm², about 65.0 N/mm² about 70.0 N/mm², about 75.0 N/mm² about 80.0 N/mm², about 85.0 N/mm² about 90.0 N/mm², about 95.0 N/mm², or about 100.0 N/mm². In some embodiments, the suture line comprises a yield stress of less than about 3.0 N/mm², about 3.5 N/mm², about 4.0 N/mm², about 4.5 N/mm², about 5.0 N/mm², about 5.5 N/mm², about 6.0 N/mm², about 6.5 N/mm², about 7.0 N/mm², about 7.5 N/mm², about 8.0 N/mm², about 8.5 N/mm², about 9.0 N/mm², about 9.5 N/mm², about 10.0 N/mm², about 15.0 N/mm², about 20.0 N/mm², about 25.0 N/mm², about 30.0 N/mm², about 35.0 N/mm², about 40.0 N/mm², about 45.0 N/mm², about 50.0 N/mm², about 55.0 N/mm² about 60.0 N/mm², about 65.0 N/mm² about 70.0 N/mm², about 75.0 N/mm² about 80.0 N/mm², about 85.0 N/mm² about 90.0 N/mm², about 95.0 N/mm², or about 100.0 N/mm². In some embodiments, the yield stress is measured at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days after closure. In some embodiments, the yield stress is measured at least about 42 days after closure.

In some embodiments, the increase of the mechanical tensile strength of the suture line is characterized by the formation of an amount or a type of collagen mimicking endogenous tissue at the suture line site. In some embodiments, the type of collagen comprises Type I collagen, Type III collagen, or a combination thereof. In some embodiments, Type I collagen is present with Type III collagen in a ratio of less than or equal to about 10:1. In some embodiments, Type I collagen is present with Type III collagen in a ratio of less than or equal to about 6:1. In some embodiments, Type I collagen is present with Type III collagen in a ratio of about 5:1 or less. In some embodiments, Type I collagen is present with Type III collagen in a ratio of about 1:1 to about 10:1. In some embodiments, Type I collagen is present with Type III collagen in a ratio of about 1.5:1 to about 9.5:1. In some embodiments, Type I collagen is present with Type III collagen in a ratio of about 2:1 to about 9:1. In some embodiments, Type I collagen is present with Type III collagen in a ratio of about 2.5:1 to about 8.5:1. In some embodiments, Type I collagen is present with Type III collagen in a ratio of about 3:1 to about 8:1. In some embodiments, Type I collagen is present with Type III collagen in a ratio of about 3.5:1 to about 7.5:1. In some embodiments, Type I collagen is present with Type III collagen in a ratio of about 4:1 to about 7:1. In some embodiments, Type I collagen is present with Type III collagen in a ratio of about 4.5:1 to about 6.5:1. In some embodiments, Type I collagen is present with Type III collagen in a ratio of about 5:1 to about 6:1. In some embodiments, the collagen is formed at the site of a suture line by at least about 28 days after closure (e.g. suturing) of the site of a suture line. In some embodiments, the collagen is formed at the site of a suture line by at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days after suturing.

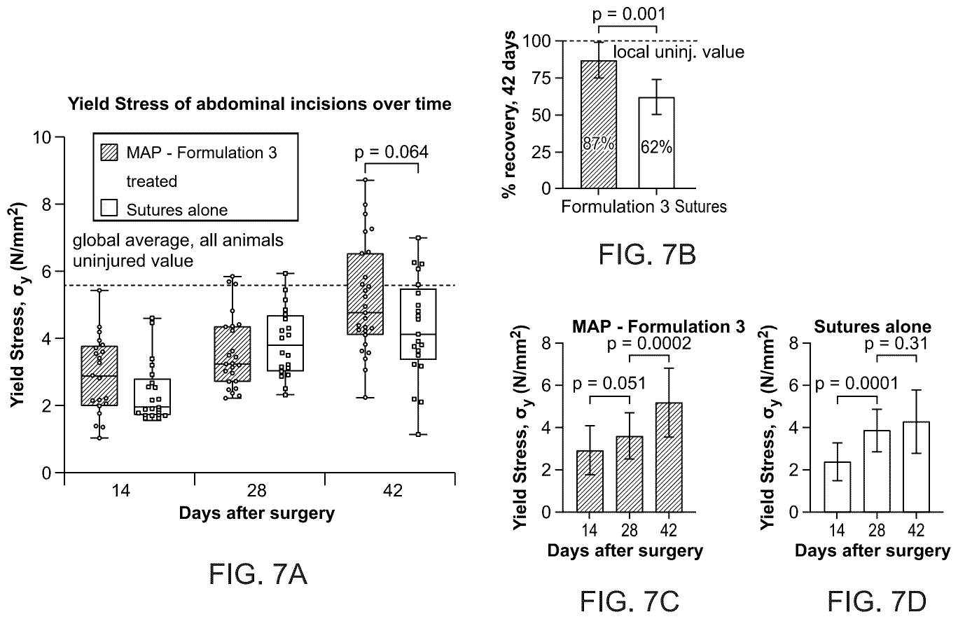

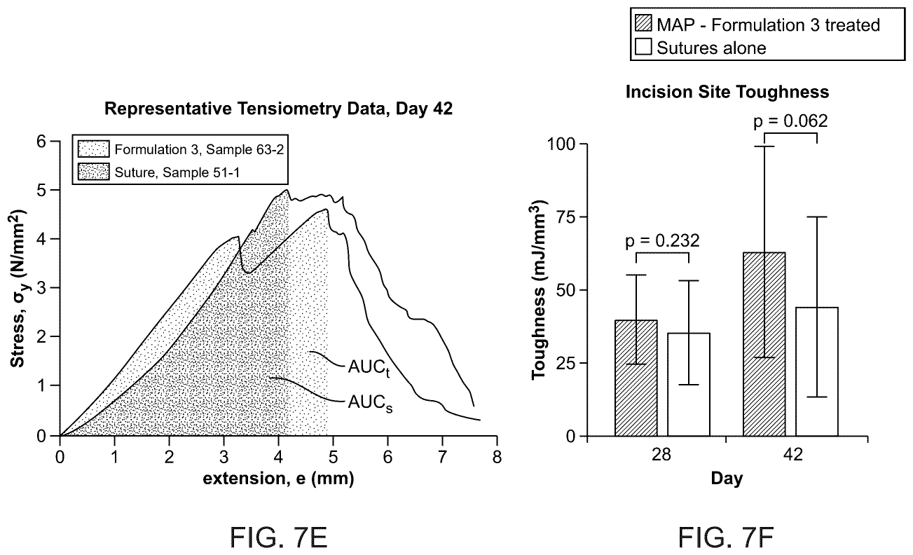

In some embodiments, the increasing the mechanical tensile strength of the suture line is characterized by increasing the toughness of the suture line. The toughness may be measured as an area under the curve of a stress versus strain curve to fracture using a tensile test (e.g., on an Instron). A nonlimiting example is depicted in FIG. 7F. In some embodiments, the suture line comprises a toughness of at least about 60.0 millijoules per millimeter cubed (mJ/mm³) at least about 42 days after closure. In some embodiments, the toughness comprises at least about 25.0 mJ/mm³, about 30.0 mJ/mm³, about 35.0 mJ/mm³, about 40.0 mJ/mm³, about 45.0 mJ/mm³, about 50.0 mJ/mm³, about 55.0 mJ/mm³, about 60.0 mJ/mm³, about 65.0 mJ/mm³, about 70.0 mJ/mm³, about 75.0 mJ/mm³, about 80.0 mJ/mm³, about 85.0 mJ/mm³, about 90.0 mJ/mm³, or about 100.0 mJ/mm³. In some embodiments, the toughness comprises no more than about 25.0 mJ/mm³, about 30.0 mJ/mm³, about 35.0 mJ/mm³, about 40.0 mJ/mm³, about 45.0 mJ/mm³, about 50.0 mJ/mm³, about 55.0 mJ/mm³, about 60.0 mJ/mm³, about 65.0 mJ/mm³, about 70.0 mJ/mm³, about 75.0 mJ/mm³, about 80.0 mJ/mm³, about 85.0 mJ/mm³, about 90.0 mJ/mm³, or about 100.0 mJ/mm³. In some embodiments, the toughness of the suture line is measured at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days after suturing. In some embodiments, the toughness of the closure is measured at least about 42 days after suturing.

In some embodiments, the suture line comprises a toughness of at least about 25.0 mJ/mm³ to about 100.0 mJ/mm³ after suturing. In some embodiments, the toughness comprises at least about 25.0 mJ/mm³ to about 100.0 mJ/mm³, about 30.0 mJ/mm³ to about 100.0 mJ/mm³, about 35.0 mJ/mm³ to about 100.0 mJ/mm³, about 40.0 mJ/mm³ to about 100.0 mJ/mm³, about 45.0 mJ/mm³ to about 100.0 mJ/mm³, about 50.0 mJ/mm³ to about 100.0 mJ/mm³, about 55.0 mJ/mm³ to about 100.0 mJ/mm³, about 60.0 mJ/mm³ to about 100.0 mJ/mm³, about 65.0 mJ/mm³ to about 100.0 mJ/mm³, about 70.0 mJ/mm³ to about 100.0 mJ/mm³, about 75.0 mJ/mm³ to about 100.0 mJ/mm³, about 80.0 mJ/mm³ to about 100.0 mJ/mm³, about 85.0 mJ/mm³ to about 100.0 mJ/mm³, or about 90.0 mJ/mm³ to about 100.0 mJ/mm³. In some embodiments, the toughness of the suture line is measured at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days after closure. In some embodiments, the toughness of the suture line is measured at least about 42 days after suturing.

In some embodiments, the increasing the mechanical tensile strength of the suture line is characterized by increasing a percent recovery of the suture line. In some embodiments, the percent recovery is calculated as the ratio of the wounded tissue tensile strength at the suture line site relative to unwounded tissue tensile strength from the same anatomical area. In some embodiments, the percent recovery of the suture line is greater than or equal to about 40% increased as compared to a reference suture line closed without the delivery of the hydrogel formulation, wherein the percent recovery is measured after suturing. In some embodiments, the percent recovery is greater than or equal to about 10%, about 20%, about 30%, about 40%, about 50%, about 60%, about 70%, about 80%, about 90%, or about 100% increased as compared to a reference suture line sutured without the delivery of the hydrogel formulation. In some embodiments, the percent recovery is measured at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days after suturing. In some embodiments, the precent recovery is measured at least about 42 days after suturing.

In some embodiments, the percent recovery of the suture line is at least about 20% to about 60% increased as compared to a reference suture line sutured without the delivery of the hydrogel formulation, wherein the percent recovery is measured after suturing. In some embodiments, the percent recovery is at least about 10% to about 50% increased, at least about 20% to about 60% increased, at least about 30% to about 70% increased, at least about 40% to about 80% increased, at least about 30% to about 90% increased, or at least about 40% to about 100% increased. In some embodiments, the percent recovery is measured at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days after suturing. In some embodiments, the percent recovery is measured at least about 42 days after suturing.

In some embodiments, the increase of the mechanical tensile strength of the suture line is characterized by increasing the yield strain of the suture line.

In some embodiments, the delivering comprises releasing the hydrogel formulation from a syringe. In some embodiments, the syringe comprises a needle. In some embodiments, the needle has a gauge comprising about 10 gauge to about 20 gauge. In some embodiments, the needle has a gauge comprising about a 18 gauge. In some embodiments, the needle has a gauge comprising about 10, 11, 12, 13, 14, 15, 16, 17, 18, 19, or 20 gauge.

In some embodiments, the syringe may have an internal volume of about 0.1 mL to about 10 mL. In some embodiments, the internal volume comprises about 5.0 mL. In some embodiments, the internal volume comprises about 0.5 mL to about 3.0 mL. In some embodiments, the volume comprises about 0.75 mL to about 2.75 mL, 1.0 mL to about 2.25 mL, 1.25 mL to about 2.0 mL, 1.0 mL to about 1.75 mL, or 1.25 mL to 1.50 mL, 0.1 mL to 5.0 mL, 5.0 mL to 10.0 mL, or 2.5 mL to 7.5 mL. In some embodiments, the syringe comprises a dose comprising greater than or equal to about 0.50 mL, 0.75 mL, 1.0 mL, 1.25 mL, 1.5 mL, 1.75 mL, 2.0 mL, 2.25 mL, 2.5 mL, 2.75 mL, or 3.0 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, or 20 mL. The dose of the hydrogel formulation may depend on the area of administration. In some embodiments, the internal volume is associated with an internal diameter of the syringe which affects the extrusion force needed to inject the hydrogel formulation compositions. In some embodiments, the internal diameters may be about 4 mm to about 9 mm. In some embodiments, the internal diameters may be about 4.5 mm to about 6.5 mm. In some embodiments, the internal diameters may be about 4.5 mm to about 8.8 mm. In some embodiments, the extrusion force needed to deliver the hydrogel formulations from the syringe is dependent on the needle gauge.

In some embodiments, methods comprise delivering the hydrogel microparticles (e.g., as disclosed herein) and the annealing agent (e.g., as disclosed herein) to the subject separately. In some embodiments, methods comprise delivering the hydrogel microparticles (e.g., as disclosed herein) and the annealing agent (e.g., as disclosed herein) to the subject together. In some embodiments, the hydrogel microparticles (e.g., as disclosed herein) and the annealing agent (e.g., as disclosed herein) have a shelf life of at least about 12 months when the hydrogel microparticles and the annealing agent are stored in a single container as a mixture. In some embodiments, the hydrogel microparticles (e.g., as disclosed herein) and the annealing agent (e.g., as disclosed herein) have a shelf life of at least about 36 months when the hydrogel microparticles and the annealing agent are stored in a single container as a mixture. In some embodiments, the shelf life of the hydrogel microparticles (e.g., as disclosed herein) and the annealing agent (e.g., as disclosed herein) as a mixture corresponds to an amount of time (e.g., 12 or 36 months) when the mixture is stored at room temperature. In some embodiments, the hydrogel microparticles (e.g., as disclosed herein) and the annealing agent (e.g., as disclosed herein) have a shelf life of at least about 12 months when the hydrogel microparticles and the annealing agent are stored in separate containers. In some embodiments, the hydrogel microparticles (e.g., as disclosed herein) and the annealing agent (e.g., as disclosed herein) have a shelf life of at least about 36 months when the hydrogel microparticles and the annealing agent are stored in separate containers. In some embodiments, the shelf life of the hydrogel microparticles (e.g., as disclosed herein) and the annealing agent (e.g., as disclosed herein) when stored in separate containers corresponds to an amount of time (e.g., 12 or 36 months) when stored at room temperature.

Disclosed herein are methods comprising administering the hydrogel formulations described herein to a tissue site of a subject. In some embodiments, the tissue site is a surgical site of a subject. In some embodiments, the tissue site is an abdominal incision. In some embodiments, the surgical site is an abdominal midline lateral incision. In some embodiments, the surgical site is an abdominal transverse incision. In some embodiments, the tissue site is anywhere comprising soft tissue. In some embodiments, the tissue site is anywhere comprising connective tissue. In some embodiments, the tissue site is anywhere comprising epithelial tissue. In some embodiments, the tissue site is anywhere comprising muscle tissue. In some embodiments, the tissue site is anywhere comprising nervous tissue. In some embodiments, methods comprise administering a dose of the hydrogel formulation to the subject, which may depend on the location and/or tissue at the tissue site as well as the intended therapeutic effect. In some embodiments, the tissue site comprises a wound site of the subject. In some embodiments, the wound site comprises a site of abrasion, avulsion, incision, laceration, puncture, or a combination thereof of the skin. In some embodiments, the wound site comprises a burn site. In some embodiments, the tissue site comprises a site of scarring (e.g., a site where a mark is left on the skin or within body tissue where a wound, burn, or sore has not healed completely and fibrous connective tissue has developed of the subject). In some embodiments, the scars are keloid, hypertrophic, contracture, adhesion, or a combination thereof.

In some embodiments, methods disclosed herein comprise delivering the hydrogel formulation to the subject under conditions sufficient for adjacent microgel particles to anneal to each other to form a stabilized scaffold. In some embodiments, the stabilized scaffold that forms is covalently stabilized (e.g., covalent interactions between the adjacent microgel particles facilitate the annealing). In some embodiments, the stabilized scaffold is porous. In some embodiments, methods comprise performing the annealing reaction of the microgel particles to form a covalently stabilized scaffold. In some embodiments, the covalently stabilized scaffold is any of the covalently stabilized scaffolds described herein. In some embodiments, methods comprise forming the covalently stabilized scaffold in a manner such that pores form between the microgel particles of the covalently stabilized scaffold (e.g., the covalently stabilized scaffold is porous).

In some embodiments, the hydrogel formulation becomes integrated with the site of a suture line in less than or equal to about 14 days following closure. In some embodiments, integration is characterized by new tissue formation in and around the porous covalently stabilized scaffold.

In some embodiments, methods disclosed herein comprise delivering the hydrogel formulation to the subject under conditions sufficient for endogenous cells of the subject to infiltrate and grow within porous covalently stabilized scaffold. In some embodiments, the cells form a cell matrix within the porous covalently stabilized scaffold. In some embodiments, the porous covalently stabilized scaffold persists at the tissue site (e.g. incision or suture line) for a length of time sufficient for the cell matrix to grow into tissue in situ. In some embodiments, the methods comprise vascularizing, depositing extracellular matrix, or producing proteins and enzymes in the tissue site that aid in treating the tissue site, or any combination thereof. In some embodiments, the methods comprise forming new tissue from the cell matrix at the injection or tissue site. In some embodiments, the new tissue is characterized by having mature vascularization, a characteristic of surrounding tissue at the tissue site, an amount or type of collagen mimicking endogenous tissue at the tissue site, or a combination thereof. In some embodiments, the characteristic of the surrounding tissue at the tissue site comprises functionally differentiated cell types from the surrounding tissue. Non-limiting examples of functionally differentiated cell types include Fibroblasts, Epithelial cells, Connective tissue cells, adipocytes, immune cells, mast cells, Langerhans cells, skeletal muscle cells, stem cells and collagen fibers. In some embodiments, the new tissue is characterized as having an extracellular matrix. In some embodiments, the hydrogel formulation partially degrades in vivo. In some embodiments, the new tissue forms and the porous covalently stabilized scaffold completely degrades by at least about 42 days after closure. In some embodiments, the new tissue is formed in addition to any tissue formed at the site of a suture line due to sutures alone. In some embodiments, the new tissue continues to remodel at the site of a suture line for at least about 42 days after closure. In some embodiments, the new tissue is formed above (e.g., superficial to) the site of a suture line and deep to (e.g., under) subcutaneous tissue. In some embodiments, the new tissue is stromal like tissue with non-aligned collagen bundles.

In some embodiments, at least part of the site of a suture line comprises elastin following degradation of the porous covalently stabilized scaffold at the site of a suture line.

In some embodiments, the at least part of the tissue site comprising the cell matrix comprises at least 25% of the tissue site following degradation of the porous covalently stabilized scaffold at the tissue site. In some embodiments, the at least part of the tissue site comprising the cell matrix comprises at least 10%, 15%, 20%, 25%, 30%, 35%, 40%, 45%, 50%, 55%, 60%, 65%, 75%, 80%, 85%, 90%, or 95% or greater of the tissue site following degradation of the porous covalently stabilized scaffold at the tissue site. In some embodiments, the at least part of the tissue site comprising the cell matrix comprises at least about 10% to about 50% of the tissue site following degradation of the covalently stabilized scaffold at the tissue site. In some embodiments, the at least part of the tissue site comprising the cell matrix comprises at least about 15% to about 45% of the tissue site following degradation of the covalently stabilized scaffold at the tissue site. In some embodiments, the at least part of the tissue site comprising the cell matrix comprises at least about 20% to about 40% of the tissue site following degradation of the covalently stabilized scaffold at the tissue site. In some embodiments, the at least part of the tissue site comprising the cell matrix comprises at least about 25% to about 35% of the tissue site following degradation of the covalently stabilized scaffold at the tissue site. In some embodiments, the at least part of the tissue site comprising the cell matrix comprises at least about 10% to about 90% of the tissue site following degradation of the covalently stabilized scaffold at the tissue site. In some embodiments, the at least part of the tissue site comprising the cell matrix comprises at least about 15% to about 90% of the tissue site following degradation of the covalently stabilized scaffold at the tissue site. In some embodiments, the at least part of the tissue site comprising the cell matrix comprises at least about 20% to about 85% of the tissue site following degradation of the covalently stabilized scaffold at the tissue site. In some embodiments, the at least part of the tissue site comprising the cell matrix comprises at least about 25% to about 80% of the tissue site following degradation of the covalently stabilized scaffold at the tissue site. In some embodiments, the at least part of the tissue site comprising the cell matrix comprises at least about 30% to about 75% of the tissue site following degradation of the covalently stabilized scaffold at the tissue site. In some embodiments, the at least part of the tissue site comprising the cell matrix comprises at least about 35% to about 70% of the tissue site following degradation of the covalently stabilized scaffold at the tissue site. In some embodiments, the at least part of the tissue site comprising the cell matrix comprises at least about 40% to about 65% of the tissue site following degradation of the covalently stabilized scaffold at the tissue site. In some embodiments, the at least part of the tissue site comprising the cell matrix comprises at least about 45% to about 60% of the tissue site following degradation of the covalently stabilized scaffold at the tissue site. In some embodiments, the at least part of the tissue site comprising the cell matrix comprises at least about 50% to about 55% of the tissue site following degradation of the covalently stabilized scaffold at the tissue site. In some embodiments, methods comprise growing cells within the porous stabilized scaffold in less than or equal to about one day following the delivering. In some embodiments, methods comprise forming the cell matrix within the porous stabilized scaffold in less than or equal to about 30 days following the delivering. In some embodiments, the cell matrix begins to form within the scaffold within 7 days after administration.

In some embodiments, methods of delivering the hydrogel formulation disclosed herein minimize a foreign body response elicited by the subject in response to the hydrogel formulation. In some embodiments, the porous covalently stabilized scaffold is effective to strengthen the suture line while minimizing a foreign body response in the subject. In some embodiments, the foreign body response is characterized by chronic inflammation. In some embodiments, the foreign body response is characterized by granuloma formation. In some embodiments, the foreign body response is characterized by scar tissue formation. In some embodiments, the foreign body response is characterized by nodule formation. In some embodiments, the foreign body response is characterized by swelling, pain, or any combination thereof. In some embodiments, the chronic inflammation, granuloma formation, nodule formation, swelling, pain or anything combination is localized to, or around, the tissue site. In some embodiments, the chronic inflammation, granuloma formation, nodule formation, swelling, pain or anything combination is localized to, or around, the site of a suture line. In some embodiments, the foreign body response is caused at a location other than the tissue site. In some embodiments, the foreign body response is characterized by a presence of multinucleate giant cells (MNGCs) (e.g., fusion of monocytes or macrophages) at the suture line of the subject. In some embodiments, the foreign body response is characterized by the persistence of MNGCs over an extended period of time. In some embodiments, the period of time comprising greater than or equal to about 1, 2, 3, or 4 weeks or more. In some embodiments, the period of time comprises greater than or equal to about 1, 2, 3, 4, 5, 6, 7, 8, 9, 10, 11, or 12 months or more. In some embodiments, the period of time comprises greater than or equal to about 1, 2, 3, 4, 5, 6, 7, 8, 9, 10, 11, or 12 years or more. In some embodiments, minimizing the foreign body response is characterized as avoiding any formation of MNGCs at the tissue site. In some embodiments, minimizing the foreign body response is characterized as the absence of MNGCs at the tissue site after a period of time after delivering the hydrogel formulation. In some embodiments, the period of time after delivering the hydrogel formulation comprises 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 days or less. In some embodiments, the period of time after delivering the hydrogel formulation comprises 1 to 30 days. In some embodiments, the period of time after delivering the hydrogel formulation comprises 1 to 29 days. In some embodiments, the period of time after delivering the hydrogel formulation comprises 1 to 28 days. In some embodiments, the period of time after delivering the hydrogel formulation comprises 1 to 27 days. In some embodiments, the period of time after delivering the hydrogel formulation comprises 1 to 26 days. In some embodiments, the period of time after delivering the hydrogel formulation comprises 1 to 25 days. In some embodiments, the period of time after delivering the hydrogel formulation comprises 1 to 24 days. In some embodiments, the period of time after delivering the hydrogel formulation comprises 1 to 23 days. In some embodiments, the period of time after delivering the hydrogel formulation comprises 1 to 22 days. In some embodiments, the period of time after delivering the hydrogel formulation comprises 1 to 21 days. In some embodiments, the period of time after delivering the hydrogel formulation comprises 1 to 20 days. In some embodiments, the period of time after delivering the hydrogel formulation comprises 1 to 19 days. In some embodiments, the period of time after delivering the hydrogel formulation comprises 1 to 18 days. In some embodiments, the period of time after delivering the hydrogel formulation comprises 1 to 17 days. In some embodiments, the period of time after delivering the hydrogel formulation comprises 1 to 16 days. In some embodiments, the period of time after delivering the hydrogel formulation comprises 1 to 15 days. In some embodiments, the period of time after delivering the hydrogel formulation comprises 1 to 14 days. In some embodiments, the period of time after delivering the hydrogel formulation comprises 1 to 13 days. In some embodiments, the period of time after delivering the hydrogel formulation comprises 1 to 12 days. In some embodiments, the period of time after delivering the hydrogel formulation comprises 1 to 11 days. In some embodiments, the period of time after delivering the hydrogel formulation comprises 1 to 10 days. In some embodiments, the period of time after delivering the hydrogel formulation comprises 1 to 9 days. In some embodiments, the period of time after delivering the hydrogel formulation comprises 1 to 8 days. In some embodiments, the period of time after delivering the hydrogel formulation comprises 1 to 7 days. In some embodiments, the period of time after delivering the hydrogel formulation comprises 1 to 6 days. In some embodiments, the period of time after delivering the hydrogel formulation comprises 1 to 5 days. In some embodiments, the period of time after delivering the hydrogel formulation comprises 1 to 4 days. In some embodiments, the period of time after delivering the hydrogel formulation comprises 1 to 3 days. In some embodiments, the period of time after delivering the hydrogel formulation comprises 1 to 2 days.

In some embodiments, the foreign body response is measured by a presence of one or more types of Macrophages at the suture line of the subject. In some embodiments, the one or more types of Macrophages comprise Type 1 Macrophages and Type 2 Macrophages. In some embodiments, the Type I Macrophages are pro-inflammatory. In some embodiments, the Type 2 Macrophages are pro-regenerative. In some embodiments, the porous covalently stabilized scaffold is effective to reduce the amount of Type 1 Macrophages to be less than the number of Type 2 Macrophages.

In some embodiments, the foreign body response is measured by a presence of one or more Multinucleated Giant Cells (MNGCs) in or around the suture line of the subject. In some embodiments, the porous covalently stabilized scaffold is effective to reduce the number of MNGCs in or around the suture line compared to a surgical mesh or tissue-derived surgical patch.

In some embodiments, chronic inflammation may be characterized as the slow, long-term inflammation lasting for an extended period of time after the delivery or administration of the hydrogel formulation. In some embodiments, the chronic inflammation can last for a period of months to years. In some embodiments, the intentional foreign body response caused by the administration of biostimulators, as discussed above, results in chronic inflammation at the tissue site. In some embodiments, methods of delivering hydrogel formulations described herein minimize such chronic inflammation while permanently filling the tissue site with new tissue. In some embodiments, a granuloma may be a small area of inflammation at or around a tissue site of a subject. In some embodiments, the granuloma may be a small area/cluster of white blood cells and other tissue at the area of inflammation. In some embodiments, the intentional foreign body response caused by the administration of biostimulators, as discussed above, results in granuloma formation at or around the tissue site. In some embodiments, methods of delivering hydrogel formulations described herein minimize such granuloma formation while filling the tissue site with new tissue. In some embodiments, scar tissue is characterized by fibrous tissue having a harder and more brittle composition than normal tissue. In some embodiments, the intentional foreign body response caused by the administration of biostimulators, as discussed above, results in scar tissue formation at the tissue site. In some embodiments, methods of delivering hydrogel formulations described herein minimize such scar tissue formation while filling the tissue site with new tissue. In some embodiments, the hydrogel formulations disclosed herein minimize such scar tissue formation at least because the new tissue that is formed within the covalently stabilized scaffold mimics the tissue at or surrounding the tissue site. For example, there is less Type I collagen and more Type III collagen deposited in the cell matrix within the covalently stabilized scaffold (forming the basis of the new tissue) than scar tissue. In some embodiments, the nodules may be sites of abnormal tissue growths. In some embodiments, the intentional foreign body response caused by the administration of biostimulators, as discussed above, results in nodule formation at the tissue site. In some embodiments, methods of delivering hydrogel formulations described herein minimize such nodule formation while filling the tissue site with new tissue. In some embodiments, the foreign body response is measured by detecting an amount of granulomas at the tissue site with histological analysis and comparing the amount of granulomas at the tissue site with a reference tissue that does not contain the hydrogel formulation. In some embodiments, the foreign body response is measured by detecting an amount of scar tissue at the tissue site with histological analysis and comparing the amount of scar tissue at the tissue site with a reference tissue that does not contain the hydrogel formulation. In some embodiments, the foreign body response is measured by detecting an amount of nodules at the tissue site with histological analysis and comparing the amount of nodules at the tissue site with a reference tissue that does not contain the hydrogel formulation. In some embodiments, foreign body response is measured by detecting chronic inflammation at the tissue site with histological analysis. In some embodiments, the foreign body response is measured by detecting an amount of multinucleate giant cells (MNGC) (e.g., fusion of monocytes or macrophages) present at the tissue site with histological analysis and comparing the amount of MNGCs at the tissue site with a reference tissue that does not contain the hydrogel formulation.

In some embodiments, methods of delivering the hydrogel formulation disclosed herein under conditions sufficient to deposit an amount or type of collagen in the cell matrix at the tissue site mimicking endogenous tissue at or surrounding the tissue site (e.g. the site of a suture line). In some embodiments, methods comprise depositing an amount or type of collagen in the cell matrix at the tissue site mimicking endogenous tissue at or surrounding the tissue site. In some embodiments, the cell matrix comprises an amount or a type of collagen mimicking endogenous tissue at the tissue site. In some embodiments, the type of collagen comprises Type I collagen, Type III collagen, or a combination thereof. In some embodiments, Type I collagen is present with Type III collagen in a ratio of less than or equal to about 10:1. In some embodiments, Type I collagen is present with Type III collagen in a ratio of less than or equal to about 6:1. In some embodiments, Type I collagen is present with Type III collagen in a ratio of about 5:1 or less. In some embodiments, Type I collagen is present with Type III collagen in a ratio of about 1:1 to about 10:1. In some embodiments, Type I collagen is present with Type III collagen in a ratio of about 1.5:1 to about 9.5:1. In some embodiments, Type I collagen is present with Type III collagen in a ratio of about 2:1 to about 9:1. In some embodiments, Type I collagen is present with Type III collagen in a ratio of about 2.5:1 to about 8.5:1. In some embodiments, Type I collagen is present with Type III collagen in a ratio of about 3:1 to about 8:1. In some embodiments, Type I collagen is present with Type III collagen in a ratio of about 3.5:1 to about 7.5:1. In some embodiments, Type I collagen is present with Type III collagen in a ratio of about 4:1 to about 7:1. In some embodiments, Type I collagen is present with Type III collagen in a ratio of about 4.5:1 to about 6.5:1. In some embodiments, Type I collagen is present with Type III collagen in a ratio of about 5:1 to about 6:1. Type I collagen may be an indicator of scar tissue or a foreign body response having taken place in the subject. In some embodiments, the lower the ratio of Type I collagen to Type III collagen, the more the foreign body response has been minimized. In some embodiments, methods comprise minimizing the ratio of Type I collagen to Type III collagen such that new tissue can be built within a subject, making new tissue with the characteristics disclosed herein while avoiding the harms disclosed herein. For example, methods comprise permanently filling at least a part of the tissue site, while minimizing, or avoiding altogether, the foreign body response elicited as a response to biostimulators.

In some embodiments, the collagen is formed at the site of a suture line by at least about 28 days after closure (e.g. suturing) of the site of a suture line. In some embodiments, the collagen is formed at the site of a suture line by at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days after suturing.

In some embodiments, methods of delivering the hydrogel formulation disclosed herein under conditions sufficient to form elastin at the tissue site. In some embodiments, methods comprise forming elastin at the tissue site. In some embodiments, the elastin persists at or around the tissue site following complete degradation of the covalently stabilized scaffold. The presence of elastin may indicate the absence of scars or scar tissue at the tissue site and may therefore be an indicator that the foreign body response has been minimized.

In some embodiments, the hydrogel formulation is biocompatible with tissue at the tissue site as determined by one or more techniques described by ISO standard 10993, the contents of which pertaining to these techniques are hereby incorporated by reference in their entirety.

### Methods of Treatment

In some embodiments, the methods further comprise treating the tissue site of the subject by delivering the hydrogel formulation to the tissue site. In some embodiments, the hydrogel formulation is delivered on top of the suture line after the incision is sutured. In some embodiments, the hydrogel formulation is delivered into the incision while the incision is. sutured. In some embodiments, the hydrogel formulation is delivered on top of the suture line after the incision is sutured and into the suture line while the incision is sutured. In some embodiments, the hydrogel begins to anneal on top of the suture line for at least 1, 2, 3, 4,1 5, 6, 7, 8, 9, 10, 11, 12, 13, 14, 15, 16, 17, 18, 19, or 20 minutes before the dermis and subcutis of the subject is sutured.

### Surgical Incision

In some embodiments, the methods comprise delivering the microgel to a suture line. In some embodiments, the suture line was an incision. In some embodiments, the incision was a surgical incision. In some embodiments, the surgical incision was an abdominal fascial incision, an umbilical incision of the abdominal fascia, a hernia or dehiscence of a previous incision in that location, a laparoscopic keyhole incision of the abdominal fascia, an inguinal incision of the abdominal fascia, or a combination thereof. In some embodiments, the incision comprises a midline abdominal incision or a transverse incision of the fascia. In some embodiments, the abdominal incision was below the dermis and subcutis of the subject.

In some embodiments, the surgical incision was an abdominal fascial incision (e.g. a midline or transverse incision of the fascia). In some embodiments, the incision was a result of a surgery performed on the subject comprising: a gastro-intestinal cancer procedure, a hysterectomy, an ovarian cancer procedure, a spinal fusion, an abdominal trauma surgery, or a combination thereof. In some embodiments, the incision was a result of a surgery performed on the subject comprising a gastro-intestinal cancer procedure. In some embodiments, the incision was a result of a surgery performed on the subject comprising a hysterectomy. In some embodiments, the incision was a result of a surgery performed on the subject comprising an ovarian cancer procedure. In some embodiments, the incision was a result of a surgery performed on the subject comprising a spinal fusion. In some embodiments, the incision was a result of a surgery performed on the subject comprising an abdominal trauma surgery.

In some embodiments, the surgical incision was an umbilical incision of the abdominal fascia. In some embodiments, the umbilical incision was an abdominal wall midline or transverse incision of the fascia. In some embodiments, the umbilical incision was below the dermis and subcutis of the subject. In some embodiments, the incision was a result of a surgery performed on the subject comprising: a gastro-intestinal cancer procedure, a hysterectomy, an ovarian cancer procedure, a spinal fusion, an abdominal trauma surgery, or a combination thereof. In some embodiments, the incision was a result of a surgery performed on the subject comprising a gastro-intestinal cancer procedure. In some embodiments, the incision was a result of a surgery performed on the subject comprising a hysterectomy. In some embodiments, the incision was a result of a surgery performed on the subject comprising an ovarian cancer procedure. In some embodiments, the incision was a result of a surgery performed on the subject comprising a spinal fusion. In some embodiments, the incision was a result of a surgery performed on the subject comprising an abdominal trauma surgery.

In some embodiments, the surgical incision was a laparoscopic keyhole incision of the abdominal fascia. In some embodiments, the laparoscopic keyhole incision was an abdominal wall midline or transverse incision of the fascia. In some embodiments, the laparoscopic keyhole incision was below the dermis and subcutis of the subject. In some embodiments, the incision was a result of a surgery performed on the subject comprising: a gastro-intestinal cancer procedure, a hysterectomy, an ovarian cancer procedure, a spinal fusion, an abdominal trauma surgery, or a combination thereof. In some embodiments, the incision was a result of a surgery performed on the subject comprising a gastro-intestinal cancer procedure. In some embodiments, the incision was a result of a surgery performed on the subject comprising a hysterectomy. In some embodiments, the incision was a result of a surgery performed on the subject comprising an ovarian cancer procedure. In some embodiments, the incision was a result of a surgery performed on the subject comprising a spinal fusion. In some embodiments, the incision was a result of a surgery performed on the subject comprising an abdominal trauma surgery.

In some embodiments, the surgical incision was an inguinal incision of the abdominal fascia. In some embodiments, the inguinal incision was an abdominal wall midline or transverse incision of the fascia. In some embodiments, the inguinal incision was below the dermis and subcutis of the subject. In some embodiments, the incision was a result of a surgery performed on the subject comprising: a gastro-intestinal cancer procedure, a hysterectomy, an ovarian cancer procedure, a spinal fusion, an abdominal trauma surgery, or a combination thereof. In some embodiments, the incision was a result of a surgery performed on the subject comprising a gastro-intestinal cancer procedure. In some embodiments, the incision was a result of a surgery performed on the subject comprising a hysterectomy. In some embodiments, the incision was a result of a surgery performed on the subject comprising an ovarian cancer procedure. In some embodiments, the incision was a result of a surgery performed on the subject comprising a spinal fusion. In some embodiments, the incision was a result of a surgery performed on the subject comprising an abdominal trauma surgery.

### Combination Treatments

In some embodiments, the methods comprise administering to the subject one or more additional agent (e.g., therapeutic agent) such as local anesthetics (e.g., lidocaine), pain medications, anti-inflammatory agents, or others that can provide a therapeutic benefit at the site of administration. In some embodiments, the microgel particles comprise one or more additional agents (e.g., drug-eluting microgel particles). In some embodiments, the microgel particles elute the one or more active agents in situ. In some embodiments, the hydrogel formulation is formulated with the one or more active agents. In some embodiments, the hydrogel formulation is not formulated with the one or more active agents, and the one or more active agents is administered separately from the hydrogel formulation. In some embodiments, the hydrogel formulation and the one or more additional active agents is administered to the subject sequentially. In some embodiments, the hydrogel formulation and the one or more additional active agents is administered to the subject substantially simultaneously.

In some embodiments, the therapeutic agent comprises a pain medication, a local anesthetic, an anti-inflammatory medication, an anti-fibrotic medication, or an antibiotic. In some embodiments the local anesthetic is ester based. In some embodiments, the ester based local anesthetic comprises benzocaine, chloroprocaine, procaine, proparacaine, tetracaine, amylocaine, or oxybuprocaine, or any combination thereof. In some embodiments, the local anesthetic is amide based. In some embodiments, the amide based local anesthetic comprises articaine, bupivacaine, dibucaine, etidocaine, levobupivacaine, lidocaine, mepivacaine, prilocaine, ropivacaine, sameridine, tonicaine, or cinchocaine, or any combination thereof. In some embodiments, the local anesthetic is or comprises lidocaine. In some embodiments, the local anesthetic consists of lidocaine. In some embodiments, the pain medication comprises codeine, fentanyl, hydrocodone, hydromorphone, meperidine, morphine, oxycodone, or tramadol, or any combination thereof. In some embodiments, the anti-inflammatory medication is a non-steroidal anti-inflammatory drug (NSAID) or a steroid. In some embodiments, the NSAID comprises ibuprofen or naproxen. In some embodiments, the steroid comprises a corticosteroid. In some embodiments, the antibiotic comprises dicloxacillin, erythromycin, or tetracycline. In some embodiments, the anti-fibrotic medication comprises pentoxifylline.

### Methods of Producing a Hydrogel Formulation

Disclosed herein, in some embodiments, are methods of producing the hydrogel formulations disclosed herein and the components thereof. In some embodiments, the methods comprise synthesizing the microgel particles from raw materials. In some embodiments, the methods comprise fine-tuning the mechanical properties of the microgel particles, hydrogel formulation, or resulting porous covalently stabilized scaffold. In some embodiments, the methods comprise purifying the microgel particles. In some embodiments, the methods comprise formulating the microgel particles into a hydrogel formulation or formulation. In some embodiments, the methods further comprise sterilizing the hydrogel formulation or formulation.

### Synthesizing Microgel Particles

Disclosed herein are methods of producing a microgel particle disclosed herein, which comprise combining raw materials (e.g., polymer, functional groups, peptides, etc.) under conditions sufficient for the individual microgel particles to form. In some embodiments, the conditions sufficient for microgel particles to form may comprise an aqueous buffer with pH ranging from 7 to 9. By way of non-limiting examples, the buffer may be phosphate buffered saline (PBS), HEPES, or Triethanolamine (TEOA). In some embodiments, the reaction may be quenched by the addition of an acid or a base to stop the reaction at a specified time after mixing and creating the water-in-oil emulsion. The reaction quenching molecule could be added to the oil phase and diffuse into the aqueous phase to quench the reaction occurring in that aqueous phase. In some embodiments, the reaction may be quenched by adding a maleimide to react with the remaining thiols. In some embodiments, the reaction may be quenched by adding an oxidizing agent to oxidize the thiols.

In some embodiments, microgel particles may be synthesized using a microfluidic device (e.g., one particle at a time per channel). In some embodiments, the microgel particles may be synthesized by water-in-oil emulsion as described in greater detail herein. In some embodiments, the microgel particles may be synthesized by water-in-oil emulsion with mechanical stirring. In some embodiments, the microgel particles may be synthesized by water-in-oil emulsion using a static mixer. In some embodiments, the microgel particles may be synthesized using in-line flow-through synthesis. In some embodiments, the microgel particles may be synthesized using a parallel production method (multiple particles at a time per channel or multiple channels in parallel).

In some embodiments, methods comprise synthesizing microgel particles by a water-in-oil emulsion process. In some embodiments, the methods begin with obtaining an oil or an oil mixture. By way of non-limiting example, the oil may be a light mineral oil (LMO), a heavy mineral oil (HMO) or a fluorinated oil. In some embodiments, oil mixtures comprise a surfactant. In some embodiments, different surfactants can be employed. In some embodiments, the surfactant may be a nonionic surfactant. Non-limiting examples of nonionic surfactants are Span80, Span20, Tween20, Tween40, Tween60, Tween80, and tocopheryl polyethylene glycol 1000 succinate (TPGS). In some embodiments, the surfactant may be an anionic surfactant. In some embodiments, the surfactant may be a fluorinated surfactant. Non-limiting examples of anionic surfactants are sodium dodecyl sulfate (SDS), sodium lauryl ether sulfate (SLES), and perfluorooctanesulfonate. In some embodiments, the surfactant may be a cationic surfactant. Non-limiting examples of cationic surfactants are cetyltrimethylammonium bromide (CTAB), and hexadecylpyridium bromide. In some embodiments, the surfactant may be an amphoteric surfactant. Non-limiting examples of amphoteric surfactants are betaine citrate, lauryl betaine, sodium, and (carboxymethyl) dimethyloleyl ammonium hydroxide. In some embodiments, the concentration of the surfactant may vary from 0.01 to 5% v/v.

In some embodiments, methods comprise adding the surfactant to the oil. In some embodiments, methods comprise adding the surfactant to the oil prior to the addition of an aqueous solution/mixture to the oil. In some embodiments, methods comprise adding the surfactant to an aqueous solution/mixture described herein. In some embodiments, having a surfactant in the aqueous phase is beneficial because if the surfactant has a high-water solubility, it is easy to remove during purification.

In some embodiments, the oil or oil mixture may be added to a bioreactor vessel through a micron filter and stirred. In some embodiments, the bioreactor vessel contains a volume from about 100 milliliters to about 1 liter. In some embodiments, the bioreactor vessel contains a volume from about 1 liter to about 10 liters. In some embodiments, the bioreactor vessel contains a volume from about 10 liters to about 100 liters. In some embodiments, the bioreactor vessel contains a volume from about 100 liters to about 1000 liters. In some embodiments, the bioreactor vessel contains a volume from about 100 liters to about 10,000 liters. In some embodiments, the bioreactor vessel contains a volume from about 10 liters to about 1000 liters. In some embodiments, the bioreactor vessel contains a volume from about 1000 liters to about 10,000 liters. In some embodiments, the micron filter has a pore size of about 0.1 μm to about 1 μm. In some embodiments, the micron filter has a pore size of about 0.2 μm.

In some embodiments, the oil or oil mixture may be added to a static mixer through a micron filter and stirred. In some embodiments, the static mixer contains a volume from about 100 milliliters to about 1 liter. In some embodiments, the static mixer contains a volume from about 1 liter to about 10 liters. In some embodiments, the static mixer contains a volume from about 10 liters to about 100 liters. In some embodiments, the static mixer contains a volume from about 100 liters to about 1000 liters. In some embodiments, the static mixer contains a volume from about 100 liters to about 10,000 liters. In some embodiments, the static mixer contains a volume from about 10 liters to about 1000 liters. In some embodiments, the static mixer contains a volume from about 1000 liters to about 10,000 liters. In some embodiments, the micron filter has a pore size of about 0.1 μm to about 1 μm. In some embodiments, the micron filter has a pore size of about 0.2 μm.

In some embodiments, methods of synthesizing microgel particles comprise providing one or more polymers as disclosed herein (e.g., in a solution). In some embodiments, the one or more polymers comprise PEG. In some embodiments, the PEG are provided in a molecular weight as disclosed herein.

In some embodiments, methods of synthesizing microgel particles comprise modifying the one or more polymers disclosed herein by attaching one or more functional groups. In some embodiments, the PEG are modified by attaching thiol and vinyl sulfone functional groups. In some embodiments, a first PEG is modified with thiol and a second PEG PEG is modified with vinyl sulfone (VS).

In some embodiments, methods of synthesizing microgel particles comprise mixing the one or more modified polymers in a solution. In some embodiments, the thiolated PEG is mixed with the PEG-VS. In some embodiments, the functional groups react to form a hydrogel (e.g., hydrogel mesh). In some embodiments, the functional groups react by a Michael addition reaction (e.g., thiol-ene Michael addition reaction). In some embodiments, methods may comprise filtering the solution. In some embodiments, the solution may comprise a peptide (e.g., cell adhesive peptide as disclosed herein). In some embodiments, the solution may comprise a buffer or buffering agent. In some embodiments, the solution may comprise a base catalyst.

In some embodiments, methods of synthesizing microgel particles comprise the methods disclosed in U.S. Pat. No. 10,912,860 or U.S. Pat. No. 10,668,185, which are incorporated herein by reference in its entirety.

### Fine-Tuning Mechanical Properties

Disclosed herein, in some embodiments, are methods of modulating the physical characteristics of the microgel particles, the covalently stabilized scaffolds, the hydrogel formulations, or any combination thereof. In some embodiments, how the physical characteristics are modulated will depend on the mode of delivery, and the subject. In some embodiments, the physical characteristics may be altered depending on the, the mode of administration, the desired biocompatibility, or any combination thereof. In some embodiments, the physical characteristic is a mechanical property of the microgel particles, the covalently stabilized scaffolds, the hydrogel formulations, or any combination thereof.

In some embodiments, methods comprise modulating the viscosity of the hydrogel, rate of degradation of the covalently stabilized scaffold, the volume fraction of the microgel particles, the pH of the microgel particles, the pH of the annealing agent solution, the pH of the covalently stabilized scaffold, the degree of substitution of the polymer, the elastic compressive modulus of the covalently stabilized scaffold, the storage modulus of the covalently stabilized scaffold, the weight percent of the polymers, the molar ratio of the functional groups, the molecular weight of the polymer, the molecular weight of the annealing agent, the additional agents (e.g., therapeutic agents), the size of the microgel particles or a combination thereof. In some embodiments, methods comprise reducing the viscosity of the hydrogel, rate of degradation of the covalently stabilized scaffold, the volume fraction of the microgel particles, the pH of the microgel particles, the pH of the annealing agent, the pH of the covalently stabilized scaffold, the degree of substitution of the polymer, the elastic compressive modulus of the covalently stabilized scaffold, the storage modulus of the covalently stabilized scaffold, the weight percent of the polymer, the molar ratio of the functional groups, the molecular weight of the polymer, the molecular weight of the annealing agent, the additional agents (e.g., therapeutic agents), or a combination thereof. In some embodiments, methods comprise increasing the viscosity of the hydrogel, rate of degradation of the covalently stabilized scaffold, the volume fraction of the microgel particles, the pH of the microgel particles, the pH of the annealing agent, the pH of the covalently stabilized scaffold, the degree of substitution of the polymer, the elastic compressive modulus of the covalently stabilized scaffold, the storage modulus of the covalently stabilized scaffold, the weight percent of the polymer, the molar ratio of the functional groups, the molecular weight of the polymer, the molecular weight of the annealing agent, the additional agents (e.g., therapeutic agents), or a combination thereof.

In some embodiments, methods comprise modulating (e.g., increasing or decreasing) the viscosity of the hydrogel or the elastic compressive modulus of the covalently stabilized scaffold. In some embodiments, methods comprise modulating the volume fraction of the microgel particles. In some embodiments, the volume fraction of the microgel particles may be about 80% to about 100%. In some embodiments, the volume fraction of the microgel particles may be about 85% to about 100%. In some embodiments, the volume fraction of the microgel particles may be about 90% to about 100%. In some embodiments, the volume fraction of the microgel particles may be about 95% to about 100%. In some embodiments, the volume fraction of the microgel particles may be at least about 90%, 91%, 92%, 93%, 94%, 95%, 96%, 97%, 98%, 99%, or 100%. In some embodiments, modulating the volume fraction of the microgel particles comprises modulating the percent concentration of microgel particles in the hydrogel. Increasing the volume fraction of the microgel particles can lead to a higher viscosity of the suspension of microgel particles hydrogel to be annealed into a covalently stabilized scaffold. Decreasing the volume fraction of the microgel particles can lead to a lower viscosity of the hydrogel to be annealed into a covalently stabilized scaffold. In some embodiments, methods comprise modulating the volume fraction of the microgel particles in a range of 0.75 mL/mL to 1.0 mL/mL to achieve a viscosity of the hydrogel of 1,000 to 1,000,000 mPa*s. Increasing the volume fraction of the microgel particles can lead to a higher elastic compressive modulus of the covalently stabilized scaffold. Decreasing the volume fraction of the microgel particles can lead to a lower elastic compressive modulus of the covalently stabilized scaffold. In some embodiments, methods comprise modulating the volume fraction of the microgel particles in a range of 0.75 mL/mL to 0.95 mL/mL to achieve an elastic compressive modulus of the covalently stabilized scaffold of about 1,000 Pa to about 17,000 Pa.

In some embodiments, methods comprise modulating the elastic compressive modulus of the microgel particles. In some embodiments, modulating the elastic compressive modulus is achieved by modulating the molar ratio of the crosslinkers, polymers (e.g., co-polymers), or combination thereof. Increasing the elastic compressive modulus of the microgel particles can lead to a higher viscosity of the hydrogel to be annealed into a covalently stabilized scaffold. Decreasing the elastic compressive modulus of the microgel particles can lead to a lower viscosity of the hydrogel to be annealed into a covalently stabilized scaffold. In some embodiments, methods comprise modulating the elastic compressive modulus of the microgel particles in a range of 15 kPa to 46 kPa to achieve a viscosity of the hydrogel of 1,000 to 1,000,000 mPa*s. Increasing the elastic compressive modulus of the microgel particles can lead to a higher elastic compressive modulus of the covalently stabilized scaffold. Decreasing the elastic compressive modulus of the microgel particles can lead to a lower elastic compressive modulus of the covalently stabilized scaffold. In some embodiments, methods comprise modulating the elastic compressive modulus of the microgel particles in a range of 15 kPa to 46 kPa to achieve an elastic compressive modulus of the covalently stabilized scaffold of about 1,000 Pa to about 17,000 Pa. In some embodiments, the microgel particles comprise an elastic compressive modulus of at least about 1,500 Pa after the annealing reaction. In some embodiments, the microgel particles comprise an elastic compressive modulus of at least about 6,000 Pa after the annealing reaction.

In some embodiments, methods comprise modulating (e.g., increasing or decreasing) the volume fraction of the microgel particles in a range of 0.75 mL/mL to 0.95 mL/mL, and modulating the elastic compressive modulus of the microgel particles in a range of 15 kPa to 46 kPa, to achieve a viscosity of the hydrogel of 1,000 to 1,000,000 mPa*s. In some embodiments, methods comprise modulating the volume fraction of the microgel particles in a range of 0.75 mL/mL to 0.95 mL/mL, and modulating the elastic compressive modulus of the microgel particles in a range of 15 kPa to 46 kPa, to achieve an elastic compressive modulus of the covalently stabilized scaffold of about 1,000 Pa to about 17,000 Pa.

In some embodiments, methods comprise modulating (e.g., increasing or decreasing) the rate of degradation of the covalently stabilized scaffold. In some embodiments, the rate of degradation is altered depending on how long it is desired for the covalently stabilized scaffold to remain at the tissue site. In some embodiments, methods comprise altering the degradation pathway, altering the polymers (e.g., co-polymers) used to make up the microgel particles, or a combination thereof to alter the rate of degradation. In some embodiments, methods comprise altering the degradation pathways to one or more of oxidative degradation, enzymatic degradation, or hydrolytic degradation. In some embodiments, methods comprise synthesizing the microgel particles with PEG to decrease the rate of degradation of the covalently stabilized scaffold. In some embodiments, methods comprise synthesizing the microgel particles without PEG to increase the rate of degradation.

In some embodiments, methods comprise modulating (e.g., increasing or decreasing) the degree of substitution of the polymer. In some embodiments, modulating the degree of substitution is achieved by increasing or decreasing the amount of functional groups to be coupled to the microgel particles. In some embodiments, modulating the molecular weights of the polymers (e.g., co-polymers) of the microgel particles alters the degree of substitution. In some embodiments, methods comprise measuring the degree of substitution using Ellman's assay, or Proton nuclear magnetic resonance (Proton-NMR).

In some embodiments, methods comprise modulating (e.g., increasing or decreasing) the elastic compressive modulus of the covalently stabilized scaffold. In some embodiments, modulating the concentration of functional groups (e.g., thiol and vinyl sulfone) included in the microgel particles can alter the elastic compressive modulus of the covalently stabilized scaffold. In some embodiments, modulating the molecular weight of the polymer(s) (e.g., co-polymer(s)) of the microgel particles can alter the concentration of functional groups (e.g., thiol and vinyl sulfone) included in the microgel particles. Increasing the concentration of the functional groups (e.g., thiolated PEG) can increase the elastic compressive modulus of the covalently stabilized scaffold. Decreasing the concentration of the functional groups (e.g., thiolated PEG) can decrease the elastic compressive modulus of the covalently stabilized scaffold. Increasing the molecular weight of the polymer(s) (e.g., PEG) can increase the elastic compressive modulus of the covalently stabilized scaffold, and decreasing the molecular weight of the polymer(s) (e.g., PEG) can decrease the elastic compressive modulus of the covalently stabilized scaffold. In some embodiments, methods comprise modulating the concentration of functional groups (e.g., thiol and vinyl sulfone) included in the gelation solution to a range of about 10 mg/mL to about 45 mg/mL to achieve an elastic compressive modulus of the covalently stabilized scaffold of about 100 Pa to about 140,000 Pa.

In some embodiments, methods comprise modulating (e.g., increasing or decreasing) how fast the covalently stabilized scaffold anneals. In some embodiments, altering the annealing agent can alter how fast the covalently stabilized scaffold anneals. In some embodiments, modulating the molecular weight of the annealing agent alters how fast the covalently stabilized scaffold anneals. For example, PEG-dithiol (PEG(SH)₂), 4-ARM-PEG-SH, and PETMA can be used to achieve an elastic compressive modulus of the covalently stabilized scaffold of about 4,000 Pa to about 7,000 Pa after about 60 minutes of annealing. In some embodiments, methods comprise modulating the pH of the annealing agent to alter how fast the covalently stabilized scaffold anneals. Increasing the pH of the annealing agent can increase how fast the covalently stabilized scaffold anneals, and decreasing the pH of the annealing agent can decrease how fast the covalently stabilized scaffold anneals. In some embodiments, methods comprise using a pH of the annealing at or below 6.5 to delay the start of the annealing reaction by 30 minutes or more. In some embodiments, methods comprise delivering a therapeutic agent (e.g., a local anesthetic (e.g., lidocaine)) to alter how fast the covalently stabilized scaffold anneals. Local anesthetics can decrease how fast the covalently stabilized scaffold anneals, and can function in hydrogel formulations with an elastic compressive modulus range from about 1,500 Pa to about 18,000 Pa. In some embodiments, methods comprise delivering a therapeutic agent (e.g., a local antibiotic (e.g. triclosan, vancomycin)) to achieve an elastic compressive modulus of the covalently stabilized scaffold of about 2,000 Pa to about 12,000 Pa after about 250 minutes of annealing.

### Purifying Microgel Particles

In some embodiments, methods comprise purifying the microgel particles. In some embodiments, methods comprise synthesizing and purifying microgel particles simultaneously. In some embodiments, methods comprise purifying microgel particles after synthesizing the microgel particles. In some embodiments, purifying microgel particles comprises performing membrane separation of the microgel particles from unwanted components. In some embodiments, different types of filtration membranes may be used (e.g., hollow fiber membranes with different pore sizes, different lumen IDs, dialysis or flat sheet membrane). In some embodiments, membrane separation comprises tangential flow filtration (TFF). In some embodiments, membrane separation comprises ultrafiltration-diafiltration (UFDF). In some embodiments, membrane separation comprises microfiltration-diafiltration (MFDF). In some embodiments, membrane separation comprises hollow-fiber-diafiltration (HFDF). TFF generally comprises a membrane filtration and separation technique. TFF may be used herein to purify and concentrate microgel particles. TFF may comprise generating a feed stream of a solution of microgel particles that passes parallel to a membrane face. In some embodiments, one portion of the solution may pass through the membrane (permeate) while the remainder (retentate) is recirculated back to the feed reservoir. This system may be referred to as diafiltration. This system may allow molecules (in the permeate) smaller than the membrane pores to move toward and through the membrane while the larger molecules, such as the microgel particles, remain in the retentate. In some embodiments, the flow in the filtration system may be controlled by a peristaltic pump. In some embodiments, the flow in the filtration system may be controlled by a Quattroflow pump or any positive displacement pump. In some embodiments, the filtration system may be closed to surrounding environment. In some embodiments, the filtration system may be open to surrounding environment.

In some embodiments, methods of purifying may comprise removing excess oil from the microgel particles. In some embodiments, methods of purifying may comprise dispersing the particles in an alcohol solution. In some embodiments, the alcohol solution comprises alcohol and water, and the alcohol is present in the solution at a ratio greater than or equal to about 0.8:1. In some embodiments, the alcohol solution comprises alcohol and water, and the alcohol is present in the solution at a ratio greater than or equal to about 0.5:1, 0.6:1, 0.7:1, 0.8:1, 0.9:1, or 1:1. In some embodiments, the alcohol solution comprises alcohol and water, and the alcohol is present in the solution at a ratio less than or equal to about 0.5:1, 0.6:1, 0.7:1, 0.8:1, 0.9:1, or 1:1. In some embodiments, the alcohol solution comprises alcohol and water, and the alcohol is present in the solution at a ratio of about 0.5:1 to about 1:1. In some embodiments, the alcohol solution comprises alcohol and water, and the alcohol is present in the solution at a ratio of about 0.6:1 to about 0.9:1. In some embodiments, the alcohol solution comprises alcohol and water, and the alcohol is present in the solution at a ratio of about 0.7:1 to about 0.8:1. In some embodiments, methods of purifying may comprise removing excess oil and surfactant that are not miscible in water while keeping the particles (mainly composed of water) dispersed and sufficiently swollen and ensuring no particle aggregation. In some embodiments, methods of purifying may comprise slowly transferring the particles into an aqueous buffer while preventing the surfactant from precipitating. In some embodiments, transferring rate may be linked to the flux of filtrate passing through the membrane, and occur at a rate of about 1 to about 1000 LMH (liters/m²/h). In some embodiments, transferring may occur at a rate of about 100 to about 500 LMH. In some embodiments, transferring may occur at a rate of about 200 to about 300 LMH. This transition rate may be particularly important to ensure that a surfactant does not precipitate on to (and within) the microgel particles, rendering the particles unsuitable for a microporous scaffold. In some embodiments, the transition rate may achieve at least one of (i) particle hydrogel mesh swelling, which is a product of the affinity for certain solvents for a given hydrogel polymer backbone/crosslinker system, and (ii) solubility of the surfactant in the continuous phase outside of the particle.

In some embodiments, methods comprise concentrating the microgel particles in a solution or suspension. In some embodiments, methods comprise: pumping the microgel particles through a membrane filtration system while a continuous phase volume is removed; continually concentrating the microgel particles at a controlled membrane flux; and maintaining a wall shear stress inside the membrane filtration system. In some embodiments, the membrane filtration system is selected from tangential flow filtration (TFF), ultrafiltration-diafiltration (UFDF), microfiltration-diafiltration (MFDF), or hollow-fiber-diafiltration (HFDF). In some embodiments, the membrane flux is controlled between 100 and 1000 L/m²/h. In some embodiments, the wall shear stress is maintained between 100 s-1 and 10,000 s-1.

### Preserving Microgel Particles

Disclosed herein, in some embodiments, are methods of preserving the microgel particles, annealing agents, additional active agents, therapeutic agents, hydrogel formulations or formulations, or a combination thereof. In some embodiments, the methods comprise preserving the microgel particles, annealing agents, additional active agents, therapeutic agents, or any combination thereof before formulating into a hydrogel formulation. In some embodiments, the preserving is performed prior to administration of the hydrogel formulation to a subject. In some embodiments, methods of preservation comprise lyophilization, cryodehydration, cryohibernation, or cryopreservation, or a combination thereof.

In some embodiments, the lyophilization of the microgel particles, annealing agents, therapeutic agents, or a combination thereof comprises the use of lyoprotectants for retaining the functionality of the microgel particles, annealing agents, therapeutic agents, or a combination thereof. Lyoprotectant comprises addition of reagents, salts, or additives that protects the microgel particles, annealing agents, therapeutic agents, or a combination thereof during the desiccation process. Common lyoprotectants include isopropanol, ethanol, glycerol, trehalose, DMSO, methylcellulose, sucrose, antioxidants, human or animal serum proteins, and cellular stress proteins. Additionally, methods for increasing the transport of lyoprotectants inside the microgel particles, annealing agents, therapeutic agents, or a combination thereof in suspension can be utilized as a way of improving the viability and function of the microgel particles, annealing agents, therapeutic agents, or a combination thereof after lyophilization. These methods include electroporation, and the addition of reagents. In some embodiments, the lyophilized microgel particles, annealing agents, therapeutic agents, or a combination thereof, can be reconstituted for delivery to a tissue site of a subject. In some embodiments, reconstitution is accomplished by introducing a reconstitution medium to the lyophilized microgel particles, annealing agents, therapeutic agents, or a combination thereof.

In some embodiments, the microgel particles are flash frozen. In some embodiments, the microgel particles are flash frozen with liquid nitrogen. In some embodiments, the microgel particles are frozen at a temperature of at least about −100 C, −110 C, −120 C, −130 C, −140 C, −150 C, −160 C, −170 C, −180 C, −190 C or −200. In some embodiments, the microgel particles are frozen at a temperature of about −196 C. In some embodiments, the microgel particles are in a solution of at least about 80%, 85%, 90%, 95%, or 100% isopropanol.

In some embodiments, lyophilization occurs at a temperature of about −55 C. In some embodiments, lyophilization occurs at a temperature of less than about −50 C, −55 C, −60 C, −65 C, −70 C, −75 C, −80 C, −85 C, −90 C, −95 C, −100 C. In some embodiments, the volume fraction of the microgel particles during lyophilization is less than about 10%, 20%, 30%, 40%, 50%, 60%, 70%, 80%, 90%, or 100%.

### Kits

Disclosed herein, in some embodiments, are kits useful for delivering the hydrogel formulations disclosed herein. In some embodiments, the kits disclosed herein may be used to deliver the hydrogel formulation to a tissue site in a subject. In some embodiments, the kit comprises the hydrogel formulations described herein, which can be used to perform the methods described herein. In some embodiments, the kit comprises the hydrogel particles and the annealing agent in separate containers. In some embodiments, the kit comprises the hydrogel and the annealing agent in a single container. In some embodiments, the kit also comprises a reconstitution medium as described herein to reconstitute a lyophilized hydrogel formulation (e.g., lyophilized hydrogel, annealing agent, or combination thereof).

Instructions for use may be included in the kit. Optionally, the kit also contains other useful components, such as, diluents, buffers, pharmaceutically acceptable carriers, syringes, catheters, applicators, pipetting or measuring tools, bandaging materials, or other useful paraphernalia. The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as compositions and the like. The packaging material is constructed by well-known methods, preferably to provide a sterile, contaminant-free environment. The packaging materials employed in the kit may be those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of the pharmaceutical composition. The packaging material has an external label which indicates the contents and/or purpose of the kit and its components.

In some embodiments, the kit comprises a first container and a second container. In some embodiments, the first container comprises a hydrogel formulation described herein without an annealing agent. In some embodiments, the second container comprises an annealing agent that, when in the presence of the hydrogel formulation in the first container, is capable of annealing the hydrogel formulation in situ to form a covalently stabilized scaffold. In some embodiments, the first container is separate from the second container. In some embodiments, the first container or the second container is a pre-loaded syringe. In some embodiments, the first container and the second container are sterilized using the methods of the present disclosure. In some embodiments, the kit further comprises an applicator, such as a microneedle patch or syringe. In some embodiments, the kit further comprises instructions for use of the hydrogel formulation, the annealing agent, the applicator, or any combination thereof, to deliver the hydrogel formulation and annealing agent to, on or around a surgical incision disclosed herein, and form a covalently stabilized scaffold of the present disclosure. In some embodiments, the hydrogel formulation is provided in FIG. 6G.

### Definitions

Unless defined otherwise, all terms of art, notations and other technical and scientific terms or terminology used herein are intended to have the same meaning as is commonly understood by one of ordinary skill in the art to which the claimed subject matter pertains. In some embodiments, terms with commonly understood meanings are defined herein for clarity and/or for ready reference, and the inclusion of such definitions herein should not necessarily be construed to represent a substantial difference over what is generally understood in the art.

As used in this specification and the appended claims, the singular forms “a,” “an,” and “the” include plural references unless the context clearly dictates otherwise. Any reference to “or” herein is intended to encompass “and/or” unless otherwise stated.

Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

The term “about,” as used herein, with reference to a number refers to that number plus or minus 10% of that number. The term “about” a range refers to that range minus 10% of its lowest value and plus 10% of its greatest value.

The term, “annealing agent,” as used herein, refers to an entity capable of inducing the annealing reaction between particles of the present disclosure (e.g., microparticles) to form an annealed scaffold (e.g., covalently stabilized scaffold). Non-limiting examples of annealing agents include Eosin Y, PETMA (Pentaerythritol tetrakis(2-mercaptoacetate))], Factor XIII/FactorXIIIa, molecules with two or more reactive functional groups including thiols (e.g., PEG-dithiol), divinyl sulfone, or a combination thereof. An annealing agent may not covalently participate in the linkage of the particles described herein when the annealing reaction is induced. An annealing agent may be covalently linked to the particles of the annealed scaffold when the annealing reaction is induced.

The term, “annealing component,” as used herein, refers to a substrate in an annealing reaction between microgel particles of the present disclosure (e.g., microparticles) that is bound to the microgel particles themselves. Non-limiting examples of annealing components include K or Q peptides, two or more reactive functional groups including thiol or thiol derivatives, vinyl or vinyl derivatives (e.g., vinyl sulfone), methacrylates, acrylates, amines, or a combination thereof.

The term “biocompatible,” as used herein, refers to biocompatibility as determined under the International Standard ISO 10993-1, which is hereby incorporated by reference in its entirety.

The term, “cell adhesive peptide,” or “cell adhesion peptide,” as used herein interchangeably refers to peptides capable of initiating cell adhesion to a synthetic material, such as a microgel particle. A non-limiting example of cell adhesive peptides is an RGD peptide. The cell adhesion peptide disclosed herein may be provided in Moral MEG, Siahaan T J. Conjugates of Cell Adhesion Peptides for Therapeutics and Diagnostics Against Cancer and Autoimmune Diseases. Curr. Top. Med. Chem. 2017; 17(32):3425-3443, which is hereby incorporated by reference in its entirety.

The term “cell matrix” as used herein refers to a network of proteins or other molecules that surround, support, and/or give structure to cells and tissues in the body.

The term, “elastic compressive modulus,” as used herein, refers to the stiffness of either individual microgel particles, macroscopic hydrogels, or annealed scaffolds of microgel particles. Elastic compressive modulus may be measured by compressive testing (failure or non-failure) in which an anvil of known cross-sectional area is depressed into a hydrogel, non-annealed scaffold (microgel particles), or annealed scaffold at a known distance and speed, while a force transducer attached to the anvil records the force placed on the anvil. The elastic compressive modulus may be mathematically calculated from the stress/strain curves recorded during compression testing.

The term, “crosslinker,” as used herein, refers to a reagent that participates in the crosslinking reaction of raw materials to form a microgel particle of the present disclosure (e.g., microparticles). A crosslinker is a linker with two or more reactive functional groups (e.g., thiol, vinyl sulfone, maleimide, acrylate, methacrylate, acrylamide, methacrylamide, norbornene, amine, hydroxyl). When a crosslinker is in excess in the crosslinker reaction, a crosslinker may also be an annealing component and participate with the annealing agent in an annealing reaction between particles of the present disclosure. Non-limiting examples of crosslinkers include vinyl derivatives with two or more vinyl groups (e.g., PEG-VS), thiol derivatives with two or more thiol groups (e.g., PEG-dithiol or thiolated HA), peptides with two or more cysteines (e.g., matrix metalloproteinase (MMP)-degradable crosslinker), or the combination thereof.

The term, “crosslinking,” as used herein, refers to a reaction to form the microgel particle of the present disclosure (e.g., microparticles).

The term “derivative” in reference to a “vinyl” or a “thiol” refers to a vinyl-containing chemical entity or a thiol-containing chemical entity, respectively. Non-limiting examples of vinyl derivatives include PEG-VS, PEG-acrylate, PEG-methacrylate, PEG-maleimide. Non-limiting examples of vinyl groups include vinyl sulfone, acrylate, methacrylate, acrylamide, maleimide, and norbornene. Non-limiting thiol derivatives include PEG-dithiol, cysteine-containing peptides (e.g., matrix metalloproteinase (MMP)-degradable crosslinker), any organosulfur compound of the form R—SH, where R represents an alkyl, or other organic substituent, methanethiol, ethanethiol, 1-propanethiol, 2-propoanethiol, allyl mercaptan, butanethiol, tert-butyl mercaptan, pentanethiols, thiophenol, dimercaptosuccinic acid, thioacetic acid, coenzyme A, glutathione, metallothionein, cysteine, 2-mercaptoethanol, dithiothreitol, dithioerythritol, 1-mercaptoindole, grapefruit mercaptan, furan-2-ylmethanethiol, 3-mercaptopropane-1, 2-diol, 3-mercapto-1-propanesulfonic acid, 1-hexadecanethiol, pentachlorobenzenethiol, or a combination thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term, “elastic modulus,” as used herein, refers to a mechanical property of a substance related to resistance of being deformed elastically when stress is applied to it, which may be calculated with the following equation:

$$δ {=}^{def} \frac{stress}{strain} ,$$

where stress is the force causing the deformation divided by the area to which the force is applied and strain is the ratio of the change in some parameter caused by the deformation to the original value of the parameter. Since strain is a dimensionless quantity, the units of δ′ will be the same as the units of stress. Elastic modulus may be measured by mechanical testing (failure or non-failure) in which a force transducer is attached to a specimen in a manner that creates a mechanical continuum between the transducer and the specimen. The specimen may then be deformed either by compressing it, stretching it, or shearing it, and the anvil records the force placed on the anvil by the specimen as it deforms. The elastic modulus may be mathematically calculated from the stress/strain curves recorded during mechanical testing. Different types of elastic moduli may be measured based on the type of deformation of the specimen. In compressive deformation, the compressive modulus (e.g. “elastic compressive modulus”) may be calculated. In stretching, the tensile modulus is calculated. In shear deformation, the shear modulus may be calculated.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay or on a piece of tissue that has been excised (removed) from a subject.

The term “foreign body response,” as used herein, refers to a fibrotic response typically resulting from an implant or hydrogel formulation that is characterized, for example, by chronic inflammation, granuloma formation, and/or scar tissue formation, at or around the site of delivery. A foreign body response can be detected in a subject by histological analysis of the tissue at or around the site of implantation, and comparing the results of the histological analysis with histology of a reference tissue that does not contain the implant or hydrogel formulation.

The term “gel,” as used herein, refers to three-dimensional network of crosslinked polymers swollen in a solvent.

The term “HEPES,” as used herein, refers to 4-(2-hydroxyethyl)-1-piperazineethanesulfonic acid.

The term “in situ,” as used herein, refers to the original site of delivery or administration, confined to the site of original site without the invasion of neighboring tissues.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

The term “in vivo” is used to describe an event that takes place in a subject's body.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J Mol Biol. 1990 Oct. 5; 215(3):403-10; Nucleic Acids Res. 1997 Sep. 1; 25(17):3389-402). Percent homology of sequences can be determined using the most recent version of BLAST, as of the filing date of this application. Percent identity of sequences can be determined using the most recent version of BLAST, as of the filing date of this application.

The term “hydrogel,” as used herein, refers to a gel that is water-insoluble and capable of holding water.

The term, “K peptide,” as used herein, refers to a peptide comprising an amino acid sequence comprising one or more lysine residues that serve as a substrate for an annealing agent in an annealing reaction, or which may assist in the crosslinking of the microgel particles disclosed herein.

The term “microparticle” or “microsphere,” as used herein, refer interchangeably to a particle that is about 0.1 and about 1000 μm in size.

The term, “microgel particle,” as used herein, refers to a particle comprised of gel that is about 0.1 and about 1000 μm in size.

The term “particle,” as used herein, refers to a singular unit of a larger system, such as, for example, the hydrogel formulation or compositions disclosed herein.

The term “percent (%) identity,” as used herein, generally refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (e.g., gaps may be introduced in one or both of the candidate and reference sequences for optimal alignment and non-homologous sequences may be disregarded for comparison purposes). Alignment, for purposes of determining percent identity, may be achieved in various ways that are known in the relevant field. Percent identity of two sequences may be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

The term “PEG,” as used herein, refers to poly(ethylene glycol).

The term “4-arm PEG-VS as used herein, refers to 4-arm poly(ethylene glycol)-vinyl sulfone.

The term “PLA,” as used herein, refers to polylactic acid or polylactide.

The term “polydispersity,” as used herein is a measure of the heterogeneity of a particle of the present disclosure (e.g., microgel particles) based on size. Polydispersity may be measured by any of laser diffraction using a particle size analyzer, dynamic light scattering, small-angle X-ray scattering (SAXS), small-angle neutron scattering (SANS), or microscopy.

The term “polymer,” as used herein, refers to a class of substance composed of macromolecules comprised of monomer repeats. Non-limiting polymers include poly(ethylene glycol) (PEG), polylactic acid (PLA), collagen, collagen, poly(methylmethacrylate) (PMMA), or any combination thereof. The polymer may be synthetic, such as PEG, PLA, PMMA, and the like. The polymer may be a modified form of the polymer, such as for example to contain one or more thiol or vinyl derivatives disclosed herein (e.g., PEG-dithiol, 4-ARM PEG-thiol, PEG-VS, thiolated HA).

The term “pore size,” as used herein, refers the size of each individual pore in a covalently stabilized scaffold defined as interstitial void space between the particles. The pore size may be measured by approximating the void area to a circle, where the diameter of each circle may be considered the size of the pore.

The term “porosity” or “void fraction,” as used herein, refer interchangeably to a measure of the void (e.g. “empty”) spaces in a material, and may be a fraction of the volume of voids over the total volume, between 0 and 1, or may be a percentage between 0% and 100%. As an example, Porosity P=Volume_{void}/Volume_{Total}. Porosity may be measured using methods disclosed in: “Void volume fraction of granular scaffolds; Lindsay Riley, Grace Wei, Yijun Bao, Peter Cheng, Katrina L. Wilson, Yining Liu, Yiyang Gong, Tatiana Segura; bioRxiv 2022.06.14.496197,” which is incorporated herein by reference in its entirety.

The term “precursor solution” refers to a solution of raw materials (e.g., polymers and/or peptides) used to form the microgel particles of the present disclosure.

The term “Q peptide,” as used herein, refers to a peptide comprising an amino acid sequence comprising one or more glutamine residues that serve as a substrate for an annealing agent in an annealing reaction, or which may assist in the crosslinking of the microgel particles disclosed herein.

The term “RGD peptide,” as used herein, refers to a peptide derived from an extracellular matrix protein having an RGD motif characterized by an amino acid sequence comprising “Arg-Gly-Asp”. Non-limiting extracellular matrix proteins include fibronectin, vitronectin, fibrinogen, von Willebrand Factor, laminin, and collagen. The RGD peptide may be provided in Moral MEG, Siahaan T J., et. al. The RGD peptide may be modified for conjugation to contain a cysteine. In some embodiments, the RGD peptide comprises an amino acid sequence comprising RGDSPGERCG (SEQ ID NO: 1).

The term “storage modulus,” as used herein, refers to a mechanical property of a viscoelastic substance related to the energy that is stored in the substance, representing its elastic portion. The storage modulus represents the ratio of the elastic stress to strain. The storage modulus of microgel particles may be measured in a surrogate nonporous gel formed with the same precursor solution that is used to make the microgel particles but that is not emulsified in an oil phase to produce microspheres. Storage modulus may be measured by undergoing a measurement of shear modulus as described above, and performing an amplitude and frequency sweep of shear stress in a parallel plate system. This will enable calculation of both the storage and the loss modulus of the viscoelastic material (together the storage and loss modulus comprise the shear modulus).

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “surrogate gel” refers to a macroscopic surrogate bulk gel made from the same precursor solution used to make a microgel particles disclosed herein.

The term “tissue site,” as used herein, refers to the discrete location of a tissue where the hydrogel formulation disclosed herein may be delivered.

As used herein, the terms “treatment” or “treating” are used in reference to a pharmaceutical or other intervention regimen for obtaining beneficial or desired results in the recipient. Beneficial or desired results include but are not limited to a therapeutic benefit, a prophylactic benefit, or an aesthetic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term, “VS,” as used herein, refers to vinyl sulfone.

As used herein, the term “closed” in reference to an incision or a suture is interchangeable with “sutured.” As used herein, the term “closed incision” is interchangeable with “suture line.”

### NUMBERED EMBODIMENTS

Embodiment 1 comprises a method of delivering a hydrogel formulation to a site of a suture line in a subject, the method comprising: delivering to the suture line site of the subject the hydrogel formulation that anneals in situ to form a porous covalently stabilized scaffold, wherein endogenous cells infiltrate the porous covalently stabilized scaffold to form a cell matrix over the suture line site of the subject. Embodiment 2 comprises the method of embodiment 1, wherein the suture line was an incision. Embodiment 3 comprises the method of embodiment 2, wherein the incision was a surgical incision. Embodiment 4 comprises the method of embodiment 3, wherein the surgical incision was an abdominal fascial incision. Embodiment 5 comprises the method of embodiment 4, wherein the abdominal fascial incision was an abdominal wall midline or transverse incision of the fascia. Embodiment 6 comprises the method of embodiment 4 or 5, wherein the abdominal incision was a result of a surgery performed on the subject comprising: a gastro-intestinal cancer procedure, a hysterectomy, an ovarian cancer procedure, a spinal fusion, an abdominal trauma surgery, or a combination thereof. Embodiment 7 comprises the method of any one of embodiments 4-6, wherein the abdominal incision was below the dermis and subcutis of the subject. Embodiment 8 comprises the method of embodiment 3, wherein the surgical incision was performed to repair a hernia or a dehiscence of a previous incision in that location. Embodiment 9 comprises the method of embodiment 3, wherein the surgical incision was an umbilical incision of the abdominal fascia. Embodiment 10 comprises the method of embodiment 9, wherein the umbilical incision was an abdominal wall midline or transverse incision of the fascia. Embodiment 11 comprises the method of embodiment 9 or 10, wherein the umbilical incision was a result of a surgery performed on the subject comprising: a gastro-intestinal cancer procedure, a hysterectomy, an ovarian cancer procedure, a spinal fusion, an abdominal trauma surgery, or a combination thereof. Embodiment 12 comprises the method of any one of embodiments 9-11, wherein the umbilical incision was below the dermis and subcutis of the subject. Embodiment 13 comprises the method of embodiment 3, wherein the surgical incision was a laparoscopic keyhole incision of the abdominal fascia. Embodiment 14 comprises the method of embodiment 13, wherein the laparoscopic keyhole incision was an abdominal wall midline or transverse incision of the fascia. Embodiment 15 comprises the method of embodiment 13 or 14, wherein the laparoscopic keyhole incision was a result of a surgery performed on the subject comprising: a gastro-intestinal cancer procedure, a hysterectomy, an ovarian cancer procedure, a spinal fusion, an abdominal trauma surgery, or a combination thereof. Embodiment 16 comprises the method of any one of embodiment 13-15, wherein the laparoscopic keyhole incision was below the dermis and subcutis of the subject. Embodiment 17 comprises the method of embodiment 3, wherein the surgical incision was an inguinal incision of the abdominal fascia. Embodiment 18 comprises the method of embodiment 17, wherein the inguinal incision was an abdominal wall midline or transverse incision of the fascia. Embodiment 19 comprises the method of embodiment 17 or 18, wherein the inguinal incision was a result of a surgery performed on the subject comprising: a gastro-intestinal cancer procedure, a hysterectomy, an ovarian cancer procedure, a spinal fusion, an abdominal trauma surgery, or a combination thereof. Embodiment 20 comprises the method of any one of embodiments 17-19, wherein the inguinal incision was below the dermis and subcutis of the subject. Embodiment 21 comprises the method of any one of embodiments 1-20, wherein the delivering comprises delivering the hydrogel formulation directly over the suture line after the suture line is sutured. Embodiment 22 comprises the method of any one of embodiments 1-21, wherein the delivering comprises delivering the hydrogel formulation into the suture line while the suture line is being sutured. Embodiment 23 comprises the method of any one of embodiments 1-22, wherein the delivering comprises delivering the hydrogel formulation into the suture line after the incision is sutured. Embodiment 24 comprises the method of embodiment 23, wherein the delivering comprises delivering the hydrogel formulation into the suture line while the suture line is being sutured and on top of the suture line after the suture line is sutured. Embodiment 25 comprises the method of any one of embodiments 21-24, wherein the hydrogel begins to anneal on top of the suture line before the dermis and subcutis of the subject is sutured. Embodiment 26 comprises the method of embodiment 25, wherein the hydrogel anneals on top of the suture line for at least about 10 minutes before the dermis and subcutis of the subject is sutured. Embodiment 27 comprises the method of any one of embodiments 1-26, wherein the delivering comprises releasing the hydrogel formulation from a syringe. Embodiment 28 comprises the method of embodiment 27, wherein the syringe comprises a needle. Embodiment 29 comprises the method of embodiment 28, wherein the needle is a blunt needle. Embodiment 30 comprises the method of embodiment 28 or 29, wherein the needle has a gauge comprising about 10 gauge to about 20 gauge. Embodiment 31 comprises the method of any one of embodiment 28-30, wherein the needle has a gauge comprising about 18 gauge. Embodiment 32 comprises the method of any one of embodiments 1-31, further comprising strengthening the suture line at the suture line site by the cell matrix formed over the suture line site. Embodiment 33 comprises the method of embodiment 32, wherein the strengthening the suture line is characterized by increasing a mechanical tensile strength of the suture line as compared to a reference suture line at an otherwise identical suture line site that was sutured without the delivery of the hydrogel formulation. Embodiment 34 comprises the method of embodiment 33, wherein the increasing the mechanical tensile strength of the suture line is characterized by the formation of an amount or a type of collagen mimicking endogenous tissue at the suture line site. Embodiment 35 comprises the method of embodiment 34, wherein the collagen is formed at the suture line site by at least about 28 days after suturing of the suture line. Embodiment 36 comprises the method of embodiment 34 or 35, wherein the collagen is formed in and around the porous covalently stabilized scaffold. Embodiment 37 comprises the method of any one of embodiments 34-36, wherein the type of collagen comprises Type I collagen, Type III collagen, or a combination thereof. Embodiment 38 comprises the method of embodiment 37, wherein Type I collagen is present with Type III collagen in a ratio of less than or equal to about 10:1. Embodiment 39 comprises the method of embodiment 37, wherein Type I collagen is present with Type III collagen in a ratio of less than or equal to about 6:1. Embodiment 40 comprises the method of embodiment 37, wherein Type I collagen is present with Type III collagen in a ratio of about 5:1 or less. Embodiment 41 comprises the method of embodiment 33, wherein the increasing the mechanical tensile strength of the suture line is characterized by increasing the yield stress of the suture line. Embodiment 42 comprises the method of embodiment 41, wherein the yield stress is calculated from a stress versus strain curve measured using a tensile test (e.g., on an Instron). Embodiment 43 comprises the method of embodiment 41 or 42, wherein the suture line comprises a yield stress of at least about 4.0 Newtons per millimeter squared (N/mm²) at least about 42 days after suturing of the suture line. Embodiment 44 comprises the method of any one of embodiments 41-43, wherein the suture line comprises a yield stress of at least about 3.0 N/mm² to about 6.0 N/mm² at least about 42 days after suturing of the suture line. Embodiment 45 comprises the method of embodiment 33, wherein the increasing the mechanical tensile strength of the suture line is characterized by increasing the toughness of the suture line. Embodiment 46 comprises the method of embodiment 45, wherein the toughness is measured as an area under the curve of a stress versus strain curve to fracture using a tensile test (e.g., on an Instron). Embodiment 47 comprises the method of embodiment 45 or 46, wherein the suture line comprises a toughness of at least about 60.0 millijoules per millimeter cubed (mJ/mm³) at least about 42 days after suturing of the suture line. Embodiment 48 comprises the method of embodiment 45 or 46, wherein the suture line comprises a toughness of at least about 25.0 mJ/mm³ to about 100.0 mJ/mm³ at least about 42 days after suturing of the suture line. Embodiment 49 comprises the method of embodiment 33, wherein the increasing the mechanical tensile strength of the suture line is characterized by increasing a percent recovery of the suture line. Embodiment 50 comprises the method of embodiment 49, wherein the percent recovery of the suture line is greater than or equal to about 40% increased as compared to a reference suture line sutured without the delivery of the hydrogel formulation. Embodiment 51 comprises the method of embodiment 50, wherein the percent recovery is measured at least about 42 days after suturing of the suture line. Embodiment 52 comprises the method of embodiment 49, wherein the percent recovery of the suture line is at least about 20% to about 60% increased as compared to a reference suture line sutured without the delivery of the hydrogel formulation. Embodiment 53 comprises the method of embodiment 52, wherein the percent recovery is measured at least about 42 days after suturing of the suture line. Embodiment 54 comprises the method of embodiment 33, wherein the increasing the mechanical tensile strength of the suture line is characterized by increasing the yield strain of the suture line. Embodiment 55 comprises the method of any one of embodiments 1-54, wherein the hydrogel formulation becomes integrated with the suture line site in less than or equal to about 14 days following suturing of the suture line. Embodiment 56 comprises the method of embodiment 55, wherein integration is characterized by new tissue formation in and around the porous covalently stabilized scaffold. Embodiment 57 comprises the method of any one of embodiments 1-56, wherein the cell matrix forms new tissue at the suture line site of the subject before complete degradation of the porous covalently stabilized scaffold. Embodiment 58 comprises the method of embodiment 57, wherein the new tissue is characterized by having (i) mature vascularization, (ii) a characteristic of surrounding tissue at the suture line site, (iii) an amount or a type of collagen mimicking endogenous tissue at the suture line site (iii) or a combination thereof. Embodiment 59 comprises the method of embodiment 58, wherein the characteristic of the surrounding tissue at the suture line site comprises functionally differentiated cell types from the surrounding tissue. Embodiment 60 comprises the method of embodiment 59, wherein: (i) the new tissue forms, and (ii) the porous covalently stabilized scaffold completely degrades by at least about 42 days after suturing the suture line. Embodiment 61 comprises the method of any one of embodiments 57-60, wherein the new tissue is formed in addition to any tissue formed at the suture line site due to sutures alone. Embodiment 62 comprises the method of any one of embodiments 57-61, wherein additional new tissue continues to form at the suture line site for at least about 42 days after suturing of the suture line. Embodiment 63 comprises the method of any one of embodiments 57-62, wherein the new tissue is formed above (e.g., superficial to) the suture line site and deep to subcutaneous tissue. Embodiment 64 comprises the method of any one of embodiments 57-63, wherein the new tissue is stromal like tissue with non-aligned collagen bundles. Embodiment 65 comprises the method of any one of embodiments 1-64, wherein the porous covalently stabilized scaffold strengthens the suture line at the suture line site of the subject while minimizing a foreign body response in the subject. Embodiment 66 comprises the method of embodiment 65, wherein the foreign body response is characterized by causing harm to the subject. Embodiment 67 comprises the method of embodiment 66, wherein the harm is characterized by causing: chronic inflammation, granuloma formation, scar tissue formation, adhesion formation, nodule formation, swelling, pain, or any combination thereof. Embodiment 68 comprises the method of embodiment 67, wherein the harm is caused at the suture line site. Embodiment 69 comprises the method of embodiment 66, wherein the porous covalently stabilized scaffold is effective to strengthen the suture line at the suture line site of the subject while minimizing a foreign body response in the subject when the foreign body response is measured by detecting an amount of granulomas at the suture line site with histological analysis and comparing the amount of granulomas at the suture line site with a reference suture line site that does not contain the hydrogel formulation. Embodiment 70 comprises the method of embodiment 66, wherein the porous covalently stabilized scaffold is effective to strengthen the suture line at the suture line site of the subject while minimizing a foreign body response in the subject when the foreign body response is measured by detecting an amount of scar tissue at the suture line site with histological analysis and comparing the amount of scar tissue at the suture line site with a reference suture line site that does not contain the hydrogel formulation. Embodiment 71 comprises the method of embodiment 66, wherein the porous covalently stabilized scaffold is effective to strengthen the suture line at the suture line site of the subject while minimizing a foreign body response in the subject when the foreign body response is measured by detecting an amount of nodules at the suture line site with histological analysis and comparing the amount of nodules at the suture line site with a reference suture line site that does not contain the hydrogel formulation. Embodiment 72 comprises the method of embodiment 66, wherein the porous covalently stabilized scaffold is effective to strengthen the suture line at the suture line site of the subject while minimizing a foreign body response in the subject when the foreign body response is measured by detecting chronic inflammation at the suture line site with histological analysis. Embodiment 73 comprises the method of embodiment 66, wherein the porous covalently stabilized scaffold is effective to strengthen the suture line at the suture line site of the subject while minimizing a foreign body response in the subject when the foreign body response is measured by a presence of one or more types of macrophages at the suture line site of the subject. Embodiment 74 comprises the method of embodiment 73, wherein the one or more types of macrophages comprise type 1 macrophages and type 2 macrophages. Embodiment 75 comprises the method of embodiment 74, wherein the type 1 macrophages are pro-inflammatory. Embodiment 76 comprises the method of embodiment 74, wherein the type 2 macrophages are pro-regenerative. Embodiment 77 comprises the method of any one of embodiments 74-76, wherein the porous covalently stabilized scaffold is effective to form more type 2 macrophages than type 1 macrophages. Embodiment 78 comprises the method of any one of embodiments 1-77, wherein the cell matrix comprises an amount or a type of collagen mimicking endogenous tissue at the suture line site. Embodiment 79 comprises the method of embodiment 78, wherein the collagen is formed at the suture line site by at least about 28 days after suturing of the suture line. Embodiment 80 comprises the method of embodiment 78 or 79, wherein the collagen is formed in and around the porous covalently stabilized scaffold. Embodiment 81 comprises the method of any one of embodiments 78-80, wherein the type of collagen comprises Type I collagen, Type III collagen, or a combination thereof. Embodiment 82 comprises the method of embodiment 81, wherein Type I collagen is present with Type III collagen in a ratio of less than or equal to about 10:1. Embodiment 83 comprises the method of embodiment 81, wherein Type I collagen is present with Type III collagen in a ratio of less than or equal to about 6:1. Embodiment 84 comprises the method of embodiment 81, wherein Type I collagen is present with Type III collagen in a ratio of about 5:1 or less. Embodiment 85 comprises the method of any one of embodiments 1-84, wherein at least part of the suture line site comprises elastin following degradation of the porous covalently stabilized scaffold at the suture line site. Embodiment 86 comprises the method of any one of embodiments 1-85, wherein the hydrogel formulation is biocompatible with tissue at the suture line site as determined by one or more techniques described by ISO standard 10993. Embodiment 87 comprises the method of any one of embodiments 1-86, wherein the porous covalently stabilized scaffold comprises a pH of about 8. Embodiment 88 comprises the method of any one of embodiments 1-87, wherein the porous covalently stabilized scaffold comprises an elastic compressive modulus of at least about 1500 Pascals (Pa) after the annealing reaction. Embodiment 89 comprises the method of any one of embodiments 1-88, wherein the hydrogel formulation comprises microgel particles comprising a cross-linked 4-arm poly(ethylene glycol) (PEG) vinyl sulfone hydrogel polymer cross-linked with a matrix metalloprotease (MMP)-degradable crosslinker, one or more K-peptides and Q-peptides, and a cell-adhesive peptide. Embodiment 90 comprises the method of embodiment 89, wherein the microgel particles are present in a suspension comprising the microgel particles and water, and the microgel particles are present in the suspension at a volume fraction of at least 90%. Embodiment 91 comprises the method of embodiment 89 or 90, wherein the hydrogel formulation further comprises PEG-dithiol, wherein the microgel particles undergo an annealing reaction to form a porous covalently stabilized scaffold after exposure to the PEG-dithiol. Embodiment 92 comprises the method of embodiment 91, wherein the PEG-dithiol comprises a molecular weight of at least about 0.5 kilodaltons (kDa) to about 10 kDa. Embodiment 93 comprises the method of embodiment 92, wherein the PEG-dithiol comprises a molecular weight of at least about 3.4 kDa. Embodiment 94 comprises the method of embodiment 91, wherein the PEG-dithiol is present in the hydrogel system in a molar concentration of at least about 0.02 millimolar (mM) to about 1.0 mM. Embodiment 95 comprises the method of embodiment 94, wherein the PEG-dithiol is present in the hydrogel system in a molar concentration of at least about 0.2 mM. Embodiment 96 comprises a hydrogel system, comprising: a) microgel particles comprising a cross-linked 4-arm poly(ethylene glycol) (PEG) vinyl sulfone hydrogel polymer cross-linked with a matrix metalloprotease (MMP)-degradable crosslinker, one or more K-peptides and Q-peptides, and a cell-adhesive peptide, wherein the microgel particles are present in a suspension comprising the microgel particles and water, wherein the microgel particles are present in the suspension at a volume fraction of at least 90%; and b) PEG-dithiol, wherein the PEG-dithiol comprises a molecular weight of at least about 0.5 kilodaltons (kDa) and wherein the microgel particles undergo an annealing reaction to form a porous covalently stabilized scaffold after exposure to the PEG-dithiol. Embodiment 97 comprises a hydrogel system, comprising: c) microgel particles comprising a cross-linked 4-arm poly(ethylene glycol) (PEG) vinyl sulfone hydrogel polymer cross-linked with a matrix metalloprotease (MMP)-degradable crosslinker, one or more K-peptides and Q-peptides, and a cell-adhesive peptide, wherein the microgel particles are present in a suspension comprising the microgel particles and water, and the microgel particles are present in the suspension at a volume fraction of at least 90%; and d) PEG-dithiol, wherein the microgel particles undergo an annealing reaction to form a porous covalently stabilized scaffold after exposure to the PEG-dithiol, wherein the PEG-dithiol comprises is present in the hydrogel system in a molar concentration of at least about 0.02 millimolar (mM) to about 1.0 mM. Embodiment 98 comprises the hydrogel system of embodiment 96 or 97, wherein the microgel particles are present in the suspension at a volume fraction of at least 90%. Embodiment 99 comprises the hydrogel system of any one of embodiments 96-98, wherein the PEG-dithiol and the 4-arm PEG vinyl sulfone are present to provide a ratio of thiol to vinyl sulfone of less than about 1.0. Embodiment 100 comprises the hydrogel system of any one of embodiments 96-99, wherein the PEG-dithiol and the 4-arm PEG vinyl sulfone are present to provide a ratio of thiol to vinyl sulfone of about 0.6 to about 1.0. Embodiment 101 comprises the hydrogel system of any one of embodiments 96-100, wherein the PEG-dithiol comprises a molecular weight of at least about 3.4 kDa. Embodiment 102 comprises the hydrogel system of any one of embodiments 96-101, wherein the PEG-dithiol is present in the hydrogel system in a molar concentration of at least about 0.2 mM. Embodiment 103 comprises the hydrogel system of any one of embodiments 96-102, wherein the microgel particles are spherical. Embodiment 104 comprises the method of any one of embodiments 96-103, wherein the microgel particles comprise microspheres. Embodiment 105 comprises the hydrogel system of any one of embodiments 96-104, wherein the microgel particles comprise diameters comprising 5 μm to 1000 μm. Embodiment 106 comprises the hydrogel system of embodiment 105, wherein the diameters comprise between 50 μm to 1000 μm. Embodiment 107 comprises the hydrogel system of embodiment 105, wherein the diameters comprise between 70 μm to 150 μm. Embodiment 108 comprises the hydrogel system of any one of embodiments 96-107, wherein the microgel particles comprise an elastic compressive modulus of at least about 500 Pascals (Pa) before the annealing reaction. Embodiment 109 comprises the hydrogel system of any one of embodiments 96-108, wherein the microgel particles comprise an elastic compressive modulus of at least about 1500 Pascals (Pa) after the annealing reaction. Embodiment 110 comprises the hydrogel system of embodiment 109, wherein the microgel particles comprise an elastic compressive modulus of at least about 6000 Pa after the annealing reaction. Embodiment 111 comprises the hydrogel system of any one of embodiments 96-110, wherein the porous covalently stabilized scaffold comprises pores comprising a median pore diameter of about 5 μm and above. Embodiment 112 comprises the hydrogel system of any one of embodiments 96-111, wherein the pores comprise a median pore diameter of about 10 μm to about 35 μm. Embodiment 113 comprises the hydrogel system of any one of embodiments 96-112, wherein the one or more cell adhesive peptides comprises an RGD peptide. Embodiment 114 comprises the hydrogel system of any one of embodiments 96-113, wherein the microgel particles comprise a polydispersity of no more than 0.1. Embodiment 115 comprises the hydrogel system of embodiment 114, wherein the polydispersity is calculated based on a standard deviation and mean size of the particles (e.g., PDI=(SD/mean)\hat{ }2). Embodiment 116 comprises the hydrogel system of any one of embodiments 96-115, wherein the hydrogel formulation further comprises a buffer, wherein the buffer comprises: a phosphate buffer, a 4-(2-hydroxyethyl)-1-piperazineethanesulfonic acid (HEPES) buffer, or an acetate buffer, or any combination thereof. Embodiment 117 comprises the hydrogel system of any one of embodiments 96-116, wherein the annealing reaction comprises a covalent synthesizing reaction. Embodiment 118 comprises the hydrogel system of embodiment 117, wherein the covalent synthesizing reaction comprises a Michael addition or a pseudo-Michael addition reaction. Embodiment 119 comprises the hydrogel system of embodiment 118, wherein the vinyl sulfone of the 4-arm PEG vinyl sulfone is a Michael acceptor in the Michael addition or pseudo-Michael addition reaction. Embodiment 120 comprises the hydrogel system of embodiment 118, wherein the thiol of the PEG-dithiol is a Michael donor in the Michael addition or pseudo-Michael addition reaction. Embodiment 121 comprises a hydrogel formulation comprising the hydrogel system of any one of embodiments 96-120 in a suspension, wherein the suspension comprises a buffer. Embodiment 122 comprises the hydrogel formulation of embodiment 121, wherein the formulation is formulated for administration to a subject. Embodiment 123 comprises the hydrogel formulation of embodiment 122, wherein the formulation is formulated for administration at a suture line of a suture line of the subject. Embodiment 124 comprises the hydrogel formulation of embodiment 122 or 123, wherein the administration minimizes a foreign body response in the subject. Embodiment 125 comprises the hydrogel formulation of any one of embodiments 121-124, wherein the formulation comprises a dose volume of about 0.01 mL to about 20 mL. Embodiment 126 comprises a delivery device comprising: a) a body comprising the hydrogel system of any one of embodiments 96-120 or the hydrogel formulation of any one of embodiments 122-126; and b) an applicator in fluidic communication with the body, wherein the delivery device is sterile. Embodiment 127 comprises the method of embodiment 126, wherein the delivery device is a syringe or needle.

### EXAMPLES

The following examples are included for illustrative purposes only and are not intended to limit the scope of the invention.

### Example 1: Tensile Strength of the Unwounded Abdominal Wall

### Methods

The cadaveric phase was conducted on euthanized animals without any surgical incision. Immediately after euthanasia, unwounded abdominal walls were taken out for tensiometry analysis (see below). No MAP test article was used for this phase. 10 animals were used.

Immediately after euthanasia, abdominal walls were taken out and cut into rectangular sections, then cut into strips horizontally (7 cm×1 cm) for tensile strength testing on an Instron. Measurements were made with digital calipers. The tissue width of area of interest was decreased manually with a biopsy punch to form a “dumbbell” shape of 2.5-3.5 mm width to ensure failure at linea alba in unwounded tissue. The stress vs strain curves were measured using a tensile test on an Instron. Yield strength of tissue (maximum force required to rip tissue) and area under the stress/strain curve (mechanical energy consumed by tissue during strain) up to the yield point were calculated. The yield strength was then normalized by the width of the tissue section at the biopsy point to account for variation in tissue thickness/area.

### Results

For the normalized yield strength of tissue across all rabbits, the coefficient of variation was 27.19% (Table 1).

TABLE 1
Normalized yield strength across all rabbits
for different locations from the xyphoid.

| analysis ranges (mm down from xyphoid) | | | | | | |
|---|---|---|---|---|---|---|
| x1 mm from | 30 | 40 | 50 | 30 | 40 | |
| xyphoid | | | | | | |
| x2 mm from | 90 | 100 | 110 | 100 | 110 | |
| xyphoid | | | | | | |
| total length of | 60 | 60 | 60 | 70 | 70 | |
| range (mm) | | | | | | |
| stats within range defined above x1 < x < x2 | | | | | | Global |
| Mean Yield | 4.8417 | 4.9353 | 4.9633 | 4.8873 | 4.9106 | 4.7600 |
| Strength (N/mm) | | | | | | |
| Standard | 1.2849 | 1.3072 | 1.3365 | 1.2681 | 1.2900 | 1.2942 |
| Deviation | | | | | | |
| CV (%) | 26.54 | 26.49 | 26.93 | 25.95 | 26.27 | 27.19 |
| standard error | 0.0214 | 0.0211 | 0.0216 | 0.0179 | 0.0179 | 0.0100 |

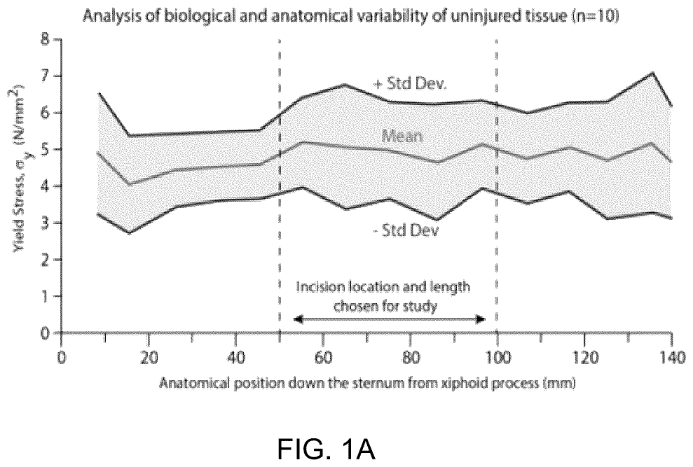

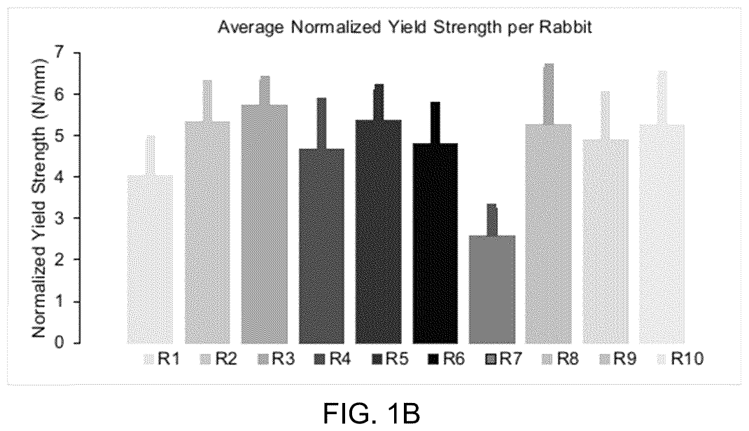

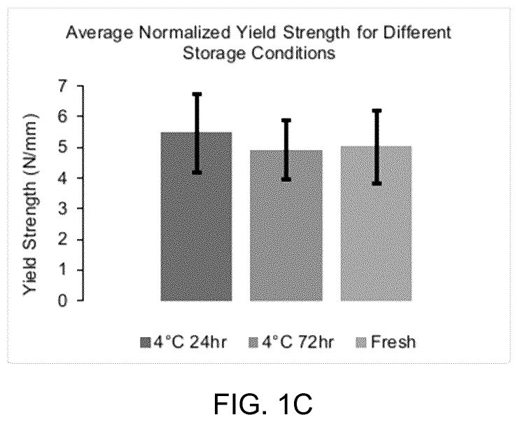

FIG. 1A shows the yield strength (normalized by width of tissue) vs abdominal position. There was no observed correlation between yield strength and abdominal position, measured from sternum/xyphoid process (cranial) towards pelvis (caudal). (R2=0.0098). Based on these data, it was decided to make a 5-cm incision about 4-5 cm from the xyphoid. FIG. 1B shows the average normalized yield strength for each rabbit. The area under the curve (AUC) up to the maximum yield strength was also calculated. However, a larger coefficient of variation (79.98%) compared to that for yield strength was observed (Table 2). Many tissue samples were observed stretching significantly before fracture. Therefore, it was decided that yield strength is a more reproducible parameter to evaluate.

TABLE 2
AUC across all rabbits for different locations from the xyphoid.

| analysis ranges (mm down from xyphoid) | | | | | | |
|---|---|---|---|---|---|---|
| x1 mm from | 30 | 40 | 50 | 30 | 40 | |
| xyphoid | | | | | | |
| x2 mm from | 90 | 100 | 110 | 100 | 110 | |
| xyphoid | | | | | | |
| total length of | 60 | 60 | 60 | 70 | 70 | |
| range (mm) | | | | | | |
| stats within range defined above x1 < x < x2 | | | | | | Global |
| Mean AUC | 2140.4602 | 2129.1174 | 1951.4950 | 2104.1470 | 2041.2491 | 2321.6623 |
| Standard Deviation | 1854.9042 | 1803.6613 | 1647.1970 | 1754.5507 | 1704.3616 | 1856.9164 |
| CV (%) | 86.66 | 84.71 | 84.41 | 83.39 | 83.50 | 79.98 |
| standard error | 42.1569 | 40.0814 | 36.6044 | 33.7414 | 32.7762 | 21.3439 |

A storage study was conducted with n=3 rabbits in the data set. Yield strengths of rabbit sections stored at +4° C. for t=24 hours, 72 hours, and fresh tissue were compared. Differences in yield strength were found to be negligible between conditions (FIG. 1C).

### Example 2: A Study to Evaluate the Use of MAP on Healing of Abdominal Midline Incisions

The overall objective of this rabbit study was to evaluate the use of MAP (Microporous Annealed Particle) product as an adjunct closure to primary suturing in an abdominal midline incision in rabbits. In the study, the effect of MAP on increased mechanical tensile strength of the incision during healing when compared to incisions closed with sutures alone was assessed.

### Methods:

The MAP formulations used are described in described in Table 3.

TABLE 3
Different MAP formulations tested in the acute phase.

| MAP | Volume | Annealing | Annealing | | |
|---|---|---|---|---|---|
| formulation | Fraction (%) | Mode | time (min) | Buffer | PH |
| 1 | 80 | Light | 3 | PBS + 5 μM | 7.4 |
| | | | | EY | |
| 2 | 100 | Light | 3 | PBS + 5 μM | 7.4 |
| | | | | EY | |
| 3 | 90 | Xlinker PEG- | 10 | PBS | 8.0 |
| | | (SH)₂ | | | |
| 4 | 90 | PRP | 10 | HEPES | 7.4 |

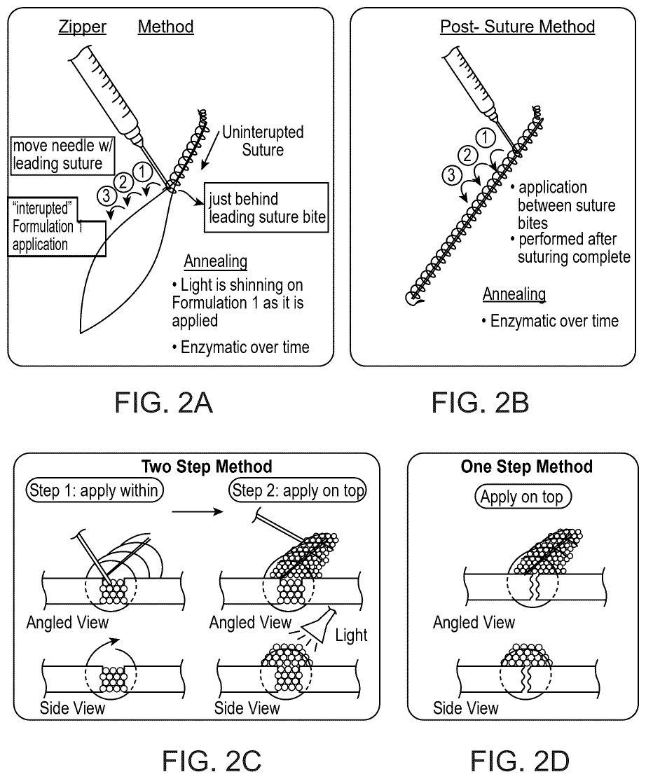

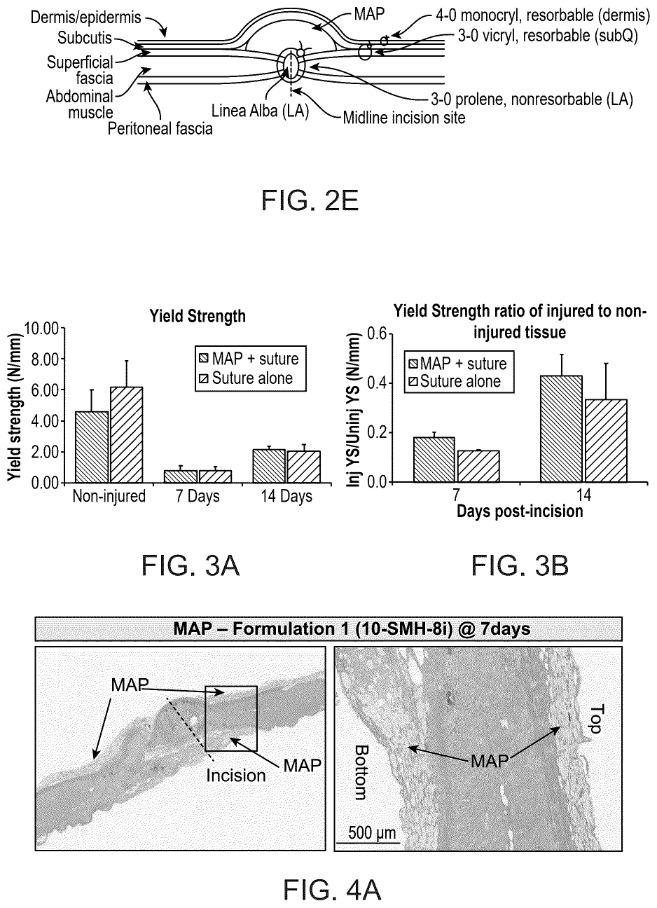

Two methods of applying MAP within the suture margin were explored, the Zipper method add the post-suture method. The Zipper method is described in FIG. 2A. MAP was applied while suturing. In the Post-suture method (FIG. 2B), MAP was applied after suturing within the incision. The MAP was applied either in the two-step method (FIG. 2C) or the one-step method (FIG. 1D). The intradermal skin was closed either on the midline or offset from the procedure. The midline closure was immediately on top of the linea alba midline incision over the subcutaneous tissue. The offset closure was 2 cm from the linea alba midline incision over the subcutaneous tissue. Table 4 below summarizes the different conditions tested in this acute phase.

TABLE 4
MAP formulation and closure techniques used in the acute phase.

| | | | MAP | Procedure # | | | | | Time |
|---|---|---|---|---|---|---|---|---|---|
| | | | volume | MAP application | Linea Alba | Dermal | Subcutaneous | Number of | points |
| Code | Description | MAP | (mL) | technique | closure | closure | closure | animals | (days) |
| SC | Control - | no | NA | NA | Continuous | Midline | Yes | 5 | 7 and |
| | Sutures only | | | | | | | | 14 |
| SC | Control - | no | NA | NA | Continuous | Offset | No | 7 | 14 |
| | Sutures only | | | | | | | | |
| SM | Zipper + top | 1 | 2 | 4 - Zipper (within | Continuous | Midline | Yes | 5 | 7 and |
| | method | | | the suture while | | | | | 14 |
| | | | | suturing) and on top | | | | | |
| SM | MAP condition | 1 | 2 | 3 - On top of | Continuous | Offset | No | 7 (2 | 14 |
| | on top | | | incision | | | | euthanized | |
| | | | | | | | | early) | |
| SMF | MAP with | 1 | 1.5 | 3 - On top of | Continuous | Offset | Yes | 1 | 14 |
| | Seprafilm over | | | incision and film | | | | | |
| | the top | | | placed on top of | | | | | |
| | | | | MAP | | | | | |
| SV | MAP at 100% | 2 | 2.5-3.0 | 3 - On top of | Continuous | Offset | No | 3 (1 | 14 |
| | volume fraction | | | incision | | | | euthanized | |
| | | | | | | | | early) | |
| SX | MAP applied | 3 | 2.5-3.0 | 3 - On top of | Continuous | Offset | No | 3 | 14 |
| | on top of the | | | incision | | | | | |
| | incision. | | | | | | | | |
| SXW | MAP applied | 3 | 2.5-3.0 | 5 - Post-suture | Continuous | Offset | No | 3 (1 | 14 |
| | within the | | | (MAP applied | | | | euthanized | |
| | incision and on | | | within suture after | | | | early) | |
| | top. | | | suturing) and on top | | | | | |
| | | | | of incision | | | | | |
| SP | MAP with PRP | 4 | 2.5-3.0 | 3 - On top of | Continuous | Offset | no | 3 | 14 |
| | | | | incision | | | | | |

The Primary Endpoint (tensiometry) included tensile tissue mechanical properties (yield stress and yield strain under unidirectional tension). The secondary endpoints (histology) included total collagen deposition, tissue inflammation and immune cell infiltrates.

TABLE 5
Microgel formulations

| | Vendor/ | | | |
|---|---|---|---|---|
| Material | Manufacturer | PN | LN | Comments |
| 1 | Tempo | 1 | DHR-020-009 | Formulated at 80% in |
| | | | 02-01-110 (was | FFB (PBS + 5 μM |
| | | | used only on 2 | EY). Annealed using |
| | | | rabbits that were | light |
| | | | euthanized early) | |
| 2 | Tempo | 2 | 02-12-002 | Formulated at 100% in |
| | | | | FFB (PBS + 5 μM |
| | | | | EY). Annealed using |
| | | | | light |
| 3 | Tempo | 3 | 02-12-002 | Formulated at 100% in |
| | | | 02-12-003 | PBS at pH 8.0, and |
| | | | | then annealed at 90% |
| | | | | with PEG-dithiol at |
| | | | | time of use |
| 4 | Tempo | 4 | 02-12-002 | Formulated at 100% in |
| | | | | HEPES at pH 7.4, and |
| | | | | then annealed at 90% |
| | | | | with PRP⁽¹⁾ |
| PEG-dithiol 3.4 | NOF | DE-034SH | M198537 | Lyophilized at 2 mM |
| kDa | | | | in water. It is |
| | | | | reconstituted in PBS at |
| | | | | pH 8.0 at time of use. |
| | | | | Final concentration in |
| | | | | MAP is 0.2 mM |

TABLE 6
Experimental set up

| | | Particle Size by LS | | | | | |
|---|---|---|---|---|---|---|---|
| | | (LS is in water) | | | Elastic Modulus (Pa) | | |
| | | Mean | Mode | SD | Before | After | Conditions for |
| PN | LN | (μm) | (μm) | (μm) | annealing | annealing | annealing |
| 1 | DHR-020- | 99.6 | 108.5 | 24.8 | 950 ± 142 | 4618 ± 1061 | Anneal with light, 10 |
| | 009 | | | | | | min at 40 cm at VF = |
| | | | | | | | 80% |
| 2 | 02-12-002 | 110.3 | 116.3 | 29.36 | 1280 | 4103 ± 810 | Anneal with light, 10 |
| | | | | | | | min at 40 cm at VF = |
| | | | | | | | 100% |
| 3 | 02-12-002 | | | | 2187 ± 834 | 9541 ± 1163 | Anneal with Cf = 0.2 |
| | | | | | | | mM PEG-dithiol (3.4 |
| | | | | | | | kDa) at VF = 90% for |
| | | | | | | | 30 min in PBS pH 8.0 |
| 4 | 02-12-002 | | | | 843 ± 114 | 5325 ± 1128 | Anneal with human |
| | | | | | | | PRP at VF = 90% for |
| | | | | | | | 30 min in HEPES pH |
| | | | | | | | 7.4. PRP was activated |
| | | | | | | | with 5% CaCl₂ (initial |
| | | | | | | | concentration). The |
| | | | | | | | final concentration of |
| | | | | | | | CaCl₂ in MAP was |
| | | | | | | | 0.05% (4.5 mM). |
| 3 | 02-12-003 | 104.5 | 105.9 | 32.21 | 514 ± 414 | 6421 ± 273 | Anneal with Cf = 0.2 |
| | | | | | | | mM PEG-dithiol (3.4 |
| | | | | | | | kDa) at VF = 90% for |
| | | | | | | | 30 min in PBS pH 8.0 |

The animal study was performed at BTS in San Diego, CA.

Animals were acclimated to wear e-collars (Mae West collar) prior to surgical procedures. This process was a multi-day process and involved progressive administration of the e-collars while providing extra enrichment for the animals. A typical e-collar acclimation regimen was as follows.
Day 1—F-Collar on for 1 hour
Day 2—F-Collar on for 3 hours
Day 3—F-Collar on for 4 hours

A Mae West collar is a long tube positioned around a rabbit's neck, fastened like a necktie with tape instead of a knot. The Mae West keeps the rabbit from reaching and pulling at the incision scar (and the staples or stitches holding it together for healing). The rabbit was still able to reach cecal pellets and groom in other areas.

Animals were anesthetized (chemically restrained) using a ketamine/xylazine injection followed by anesthesia maintenance using a gas mixture of isoflurane and oxygen for survival surgical procedures. Anesthesia was maintained using gas isoflurane/oxygen mixture. Rabbits received prophylactic analgesia (buprenorphine).

The rabbit surgical site (ventral abdomen) was prepared using clippers to remove hair, and skin was disinfected using two subsequent wipes of iodine and/or chlorohexidine solution. A sterile drape was placed to ensure cleanliness of the surgical site, and the skin was disinfected again using iodine and/or chlorohexidine followed by a sterile saline wipe.

First, an incision for epidermis, dermis and subcutis was made either in the midline or off center (2 cm) from midline along the cranio-caudal axis, a flap was created to expose Linea Alba. Blunt dissection was used to separate the subcutaneous tissue from the abdominal wall. Surgical clamps were used to ensure the site remains open and the abdominal wall midline (linea alba) is accessible for the next incision procedure.

The xyphoid process was palpated gently to determine its anatomical location and subsequently determine where to place the abdominal incision. A sterile incision and closure guide (stainless steel 304 series (0.100″), 3.543×0.787, CAD file in Tissue Marking Template 0.4 cm×0.4 cm 15 holes from midline 5 cm closure (FIG. 2E) 0.3 cm holes.SLDPRT) was placed directly on to the abdomen. A skin pen was used to draw a line where the incision should be made, and place dots where the bites for the suture closure were. The guide was then removed, leaving only the marks. Surgical tweezers were used to tent the caudal end of the incision and a #11 blade was used to start the incision. Subsequently, the incision was advanced in the cranial direction until an incision length of approximately 5 cm was reached.

Using Prolene 3-0 sutures fitted with an SH 26 mm ½ circle curved needle, closure of the incision began from the cranial end of the incision, moving in the caudal direction using a single, uninterrupted (continuous) suture. The marks made from the incision/closure guide defined where to put suture bites. This resulted in a ‘small bites’ method where each suture bite length is ~4 mm and the distance between bites is ~4 mm. A suture to incision length of 4× was used.

Here different MAP formulations were applied using different applications methods as summarized in Table 4. Formulation 3 (90% VF, annealed with PEG-dithiol crosslinker) was applied on top of the incision (procedure #3).

After MAP application, in the case of the dermal incision being made in the midline, the subcutaneous flap was closed over the abdominal wall incision using Vicryl 3-0 sutures fitted with an SH ½ circle 26 mm needle. In the case of the dermal incision being made offset from the midline, then the subcutaneous flap was partially sutured (only one side) over the abdominal wall incision using Vicryl 3-0 sutures fitted with an SH ½ circle 26 mm needle. Finally, intradermal skin closure was performed either in the midline or offset (2 cm) from midline incision over the subcutaneous tissue using Monocryl 4-0 sutures fitted with a PS-2 ⅜ circle 19 mm needle. Topical antimicrobial was applied on the sutured skin. Standard bandaging was used to further secure the incision site. During recovery the animals received 100 mL of subcutaneous fluid (Lactated Ringers). A Mae West Collar was placed around the rabbit's neck after surgery.

Analgesics were administered every 24 hours by IM injection for a 48-hour period post-surgery. The animals were monitored every day for a week and weekly after that. Animals were euthanized after 7 and 14 days.

Immediately after euthanasia, the abdominal wall was collected (subcutis was left on top of wound area during tissue collection) and placed in PBS or saline solution in a plastic box. The tissue was sectioned in multiples strips for tensiometry analysis and histology as described below.

Tensiometry: The strips for tensiometry were 10 mm×60 mm with a dumbbell of 2.5-3.5 mm diameter in the center to ensure the sample breaks at the incision site on the Instron. Non-resorbable sutures for Linea Alba were cut after necropsy, prior to mechanical analysis. The stress vs strain curves were measured using a tensile test on Instron, then the tensile strength (yield stress) was calculated.

Histology: The strips for histology were 5 mm×30 mm and were placed in a cassette fixed with 4% PFA for 24 h, then placed in 70% ethanol before shipping to Histowiz for analysis. The tissues were embedded in paraffin and then stained with Hematoxylin and Eosin (H&E) and Picrosirius red (collagen stain).

### Results

Cohort 1-1 (80%) at 7 and 14 Days Applied within and on Top of the Incision.

The first cohort of rabbits is composed of 10 rabbits (Rabbit #1-10) where Formulation 1 in conjunction of sutures was compared to sutures alone (control) 7 and 14 days after incision and treatment.

FIG. 3A shows the yield strength measured by Instron for non-injured tissue, for injured tissue treated with sutures alone and for injured tissue treated with MAP and sutures, 7 and 14 days after treatment. FIG. 3B shows the yield strength normalized to non-injured tissue, for injured tissue treated with sutures alone and for injured tissue treated with MAP (Formulation 1) and sutures. For both time points (7 and 14 days), no statistical difference in the yield strength was observed between sutures alone and MAP (Formulation 1) in conjunction of sutures.

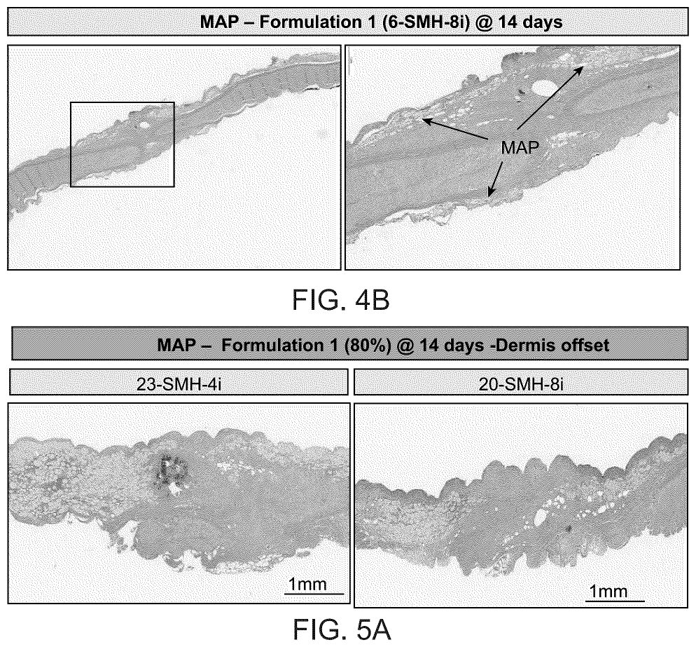

According to histology, although Formulation 1 (formulated at 80%) was applied within the suture while suturing and on top of the incision (procedure #4), no material was observed within the incision (FIG. 4A-4B). Formulation 1 was observed on top and bottom of the tissue indicating that some material went under the abdominal wall during application. Formulation 1 was not observed directly on top of the incision but rather on the right and left sides of the incision, at both times points 7 and 14 days after application indicating that Formulation 1 was not stabilized enough to remain in place.

### Cohorts 2 & 3—Alternative Methods and Alternative Map Formulations—14 Days.

The goal of the second and third cohorts of rabbits (rabbits #11-37) was to optimize both the MAP formulation and the application method so the MAP material would remain on top of the incision and ideally within the incision to maximize the chances to promote new tissue that would strengthen the injured tissue.

Rather than creating the incision of the dermis right on top of the linea alba incision, it was decided to make the incision of the dermis offset (2 cm) from the midline to minimize the disturbance of the applied material during closure of the subcutaneous and dermis layers.

Overall, closing the dermis offset from the midline helped keeping more material over the abdominal wall. This technique was used for all subsequent surgeries.

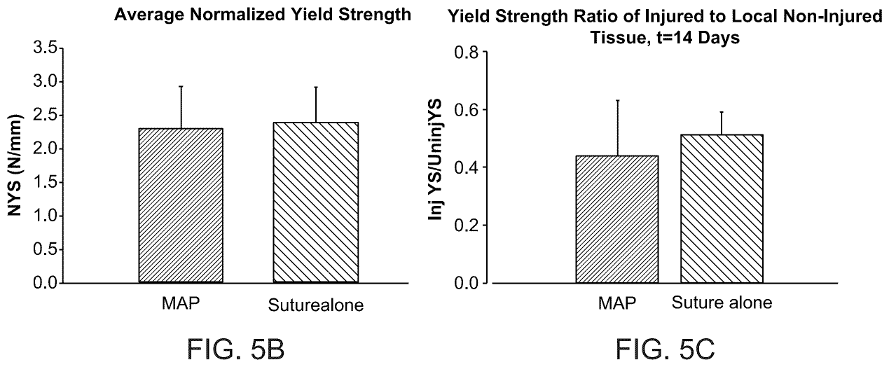

However, in the case of Formulation 1 (at 80%), the material was not directly located on top of the incision, and no evidence of new tissue formed on top of the incision was observed (FIG. 5A). Furthermore, it did not seem to improve the yield strength when compared to sutures alone (FIG. 5B-5C).

### Alternative Map Formulations

In order to maintain as much MAP on top of the incision as possible, it was decided to tune some characteristics of the MAP formulation:
Increase volume fraction from 80% to 90% (Formulation 5)
Use the PEG-dithiol crosslinker to anneal MAP in order to achieve greater elastic modulus and increase volume fraction from 80% to 90% (Formulation 3).
Use Platelet-rich plasma (PRP) to anneal MAP in order to achieve greater elastic modulus and promote growth factors production and increase volume fraction from 80% to 90% (Formulation 4).

FIG. 6A shows the stress measured on Instron for the samples collected at Day 14 for different MAP formulations. Formulation 3 (MAP Chem Xlink) demonstrated the highest tissue stress.

Although more material was observed over the abdominal wall for 1.2 when formulated at 100% (probably due to the offset dermis incision and closure) (FIG. 6B), the stress for this formulation was identical to Formulation 1 formulated at 80% (FIG. 6A). Increasing the volume fraction did not seem to have a significant effect.

A significant amount of material was observed directly on top of the incision for Formulation 4 annealed with PRP (FIG. 6C) and for Formulation 3 annealed with PEG-dithiol crosslinker (FIG. 6D). Furthermore, both formulations had slightly larger stress (FIG. 6A). Because Formulation 3 had the highest stress among all formulations, it was decided to continue with this formulation (Formulation 3) for further investigation.

### Alternative Application Method

Then, it was decided to evaluate whether Formulation 3 could be injected within the incision after closure in addition to application on top (procedure #5). In this procedure, Formulation 3 loaded in a syringe was injected using a needle between the sutures. As shown in FIG. 6E, no material was found within the incision. Although the stress is slightly higher with procedure #5 than with procedure #3 (applied only on top) (FIG. 6F), the difference was small. However, this procedure poses higher risks of puncturing organs that are present underneath the abdominal wall.

FIG. 6F shows the stress measured on Instron for the samples collected at Day 14 for different MAP formulations and different application methods. Formulation 3 (MAP Chem Xlink) added within and on top of the incision (through procedures 4 and 5) demonstrated the highest tissue stress followed by Formulation 3 added on top only. However, no statistically significant difference was observed between groups. FIG. 6G summarizes the formulation screening results.

### CONCLUSION

In this acute phase of the rabbit study, Formulation 3 was selected as the ideal candidate because it showed an increase in the tensile strength after 14 days compared to sutures alone, a significant amount of material was integrated on top of the incision, and no PRP or light was required.

### Example 3: Rabbit Abdominal Midline Study

The objective of this study was to evaluate the use of MAP (Microporous Annealed Particle) material product as an adjunct closure to primary suturing in an abdominal midline incision in rabbits. In the study, the use of MAP in increasing mechanical tensile strength of the incision during healing when compared to incisions closed with sutures alone was assessed. The histology of the incision site was also evaluated over the course of healing to examine how the material integrates with the incision site and surrounding tissue.

### Methods

TABLE 7
Materials

| | Vendor/ | | | |
|---|---|---|---|---|
| Material | Manufacturer | PN | LN | Comments |
| 3 (MAP) | Tempo | 3 | 02-12-002 | Formulated at 100% in PBS at pH |
| | | | 02-12-003 | 8.0, and then annealed at 80% |
| | | | | with PEG-dithiol at time of use. |
| PEG-dithiol | NOF | DE-034SH | M198537 | Lyophilized at 2 mM in water. It |
| 3.4 kDa | | | | is reconstituted in PBS at pH 8.0 |
| | | | | at time of use. |
| | | | | Final concentration in MAP is 0.2 |
| | | | | mM. |

TABLE 8
Experimental set up.

| | | Particle Size by LS | | | | | |
|---|---|---|---|---|---|---|---|
| | | (LS is in water) | | | Elastic Modulus (Pa) | | |
| | | Mean | Mode | SD | Before | After | Conditions for |
| PN | LN | (μm) | (μm) | (μm) | annealing | annealing | annealing |
| 3 | 02-12-002 | 110.3 | 116.3 | 29.36 | 2187 ± 834 | 9541 ± 1163 | Anneal with Cf = 0.2 |
| | | | | | | | mM PEG-dithiol (3.4 |
| | | | | | | | kDa) at VF = 90% for |
| | | | | | | | 30 min in PBS pH 8.0 |
| 3 | 02-12-003 | 104.5 | 105.9 | 32.21 | 514 ± 414 | 6421 ± 273 | Anneal with Cf = 0.2 |
| | | | | | | | mM PEG-dithiol (3.4 |
| | | | | | | | kDa) at VF = 90% for |
| | | | | | | | 30 min in PBS pH 8.0 |

7 animals per condition were used. The animal study was performed at BTS in San Diego, CA. Animals were acclimated to wear e-collars (Mae West collar) prior to surgical procedures. This process was a multi-day process and involved progressive administration of the e-collars while providing extra enrichment for the animals. A typical e-collar acclimation regimen was performed as described in Example 2.

Two veterinarians performed the surgeries over the course of the study. They both used the same surgical techniques with the exception of the tacking suturing of the subcutaneous flap as described below.

Animals were anesthetized (chemically restrained) using a ketamine/xylazine injection followed by anesthesia maintenance using a gas mixture of isoflurane and oxygen for survival surgical procedures. Anesthesia was maintained using gas isoflurane/oxygen mixture. Rabbits received prophylactic analgesia (buprenorphine).

The rabbit surgical site (ventral abdomen) was prepared using clippers to remove hair, and skin was disinfected using two subsequent wipes of iodine and/or chlorohexidine solution. A sterile drape was placed to ensure cleanliness of the surgical site, and the skin was disinfected again using iodine and/or chlorohexidine followed by a sterile saline wipe.

First, an incision for epidermis, dermis and subcutis was made off center from midline along the cranio-caudal axis, a flap was created to expose Linea Alba (FIG. 2E). Blunt dissection will be used to separate the subcutaneous tissue from the abdominal wall. Surgical clamps will be used to ensure the site remains open and the abdominal wall midline (linea alba) is accessible for the next incision procedure.

The xyphoid process was palpated gently to determine its anatomical location and subsequently determine where to place the abdominal incision. A sterile incision and closure guide was placed directly on to the abdomen. A skin pen was used to draw a line where the incision should be made, and place dots where the bites for the suture closure were. The guide was then removed, leaving only the marks. Surgical tweezers were used to tent the caudal end of the incision and a #11 blade was used to start the incision. Subsequently, the incision was advanced in the cranial direction until an incision length of approximately 5 cm is reached.

Using Prolene 3-0 sutures fitted with an SH 26 mm ½ circle curved needle, closure of the incision began from the cranial end of the incision, moving in the caudal direction using a single, uninterrupted (continuous) suture. The marks made from the incision/closure guide defined where to put suture bites. This resulted in a ‘small bites’ method where each suture bite length is ~4 mm and the distance between bites is ~4 mm. A suture to incision length of 4× was used.

Formulation 3 was selected for this full study. Formulation 3 was formulated at 90% (volume fraction). Immediately prior to MAP application, Formulation 3 was annealed at pH 8.0 using PEG-dithiol (3.4 kDa) dithiol crosslinker (Cf=0.2 mM). MAP was applied directly over the completely closed incision site and let annealed for 10 min before the next layer was sutured.

After MAP application, the subcutaneous flap was closed over the abdominal wall incision with tacking sutures made between subcutis and superficial fascia using Vicryl 3-0 sutures fitted with an SH ½ circle 26 mm needle. Finally, intradermal skin closure was performed offset from midline incision over the subcutaneous tissue using Monocryl 4-0 sutures fitted with a PS-2 ⅜ circle 19 mm needle. Topical antimicrobial was applied on the sutured skin. Standard bandaging was used to further secure the incision site. During recovery the animals received 100 mL of subcutaneous fluid (Lactated Ringers). A Mae West Collar was placed around the rabbit's neck after surgery.

Analgesics were administered every 24 hours by IM injection for a 48-hour period post-surgery. The animals were monitored every day for a week and weekly after that. Animals were euthanized after 14, 28 and 42 days.

The incision for the dermis and subcutis was off center from midline. A flap was created to expose the Linea Alba. A 5 cm Linea Alba incision was made (begins at least 4 cm caudal from xiphoid process, location of xyphoid was determined by palpation). Next, a continuous, uninterrupted suture closure of Linea Alba incision (small bites technique) was performed using the stainless-steel template (Stainless Steel 304 series (0.100″), 3.543×0.787, CAD file in Tissue Marking Template 0.4 cm×0.4 cm 15 holes from midline 5 cm closure 0.3 cm holes. The MAP material was applied directly over the closed incision. The SubQ flap was closed over the incision site, with tacking sutures made between the SubQ and superficial fascia (interrupted). Closure of dermis (intradermal closure) was on offset from midline incision.

Immediately after euthanasia, the abdominal wall was collected (subcutis was left on top of wound area during tissue collection) and placed in PBS or saline solution in a plastic box. The tissue was sectioned in multiples strips for tensiometry analysis and histology as described below.

Tensiometry: The strips for tensiometry were 10 mm×60 mm with a dumbbell of 2.5-3.5 mm diameter in the center to ensure the sample breaks at the incision site on the Instron. Non-resorbable sutures for Linea Alba were cut after necropsy, prior to mechanical analysis. The stress vs strain curves were measured using a tensile test on Instron, then the tensile strength (yield stress) was calculated.

Histology: The strips for histology were 5 mm×30 mm and were placed in a cassette fixed with 4% PFA for 24 h, then placed in 70% ethanol before shipping to Histowiz for analysis. The tissues were embedded in paraffin and then stained with H&E and Picrosirius red (collagen stain). Both stains were imaged using non-polarized light. Note that further analysis could be conducted by imaging the Picrosirius red stained slides using polarized light to distinguish between collagen I and collagen III.

### Results

Formulation 3 led to stronger incisions after 42 days (FIG. 7A-7D). Furthermore, 40% increase in recovery strength was observed for Formulation 3 compared to sutures alone. Lastly, strength continuously increased overtime for Formulation 3, while sutures alone plateaued at 42 days.

An example of stress-extension curves is shown is FIG. 7E. In addition, toughness of incision also increases with Formulation 3 compared to sutures alone (FIG. 7F.).

Representative histology images for H&E and Picrosirius red stains are shown below (FIG. 8A-8M) for both Formulation 3 and sutures alone after 14, 28 and 42 days.

Formulation 3 became integrated with the abdominal wall fascia above the incision site after 14 days. New tissue had grown into the scaffold and tissue has grown over the top of the scaffold. Formulation 3 was building new tissue that matures over the 42-day period. The MAP appeared to degrade over the 42 day period, during which residual macrophages could be seen in the places where scaffold likely was prior to fixation and staining.

This new tissue is ‘in addition’ to what is created in the wound with sutures alone. Primarily this new tissue appeared to be ‘above’ or superficial to the abdominal incision site, and deep to the subcutaneous tissue. In MAP—tissue continued to remodel through the 42-day period. Significant cellular infiltration into the scaffold with mature blood vessels and significant collagen deposition was observed by day 28, and complete scaffold degradation and tissue infiltration was observed by day 42.

Formulation 3 produced stronger abdominal midline incisions when used as adjunct to suture closure. This was corroborated by new collagen deposition in scaffold that matures over time. The magnitude of the 40% change in recovery above control indicates potential clinical significance in reducing herniation rates.

While preferred embodiments of the present subject matter have been shown and described herein, it will be obvious to those skilled in the art that such embodiments are provided by way of example only. Numerous variations, changes, and substitutions will now occur to those skilled in the art without departing from the present subject matter. It should be understood that various alternatives to the embodiments of the present subject matter described herein may be employed in practicing the present subject matter.

1. A method of delivering a hydrogel formulation to a site of a suture line in a subject, the method comprising: delivering to the suture line site of the subject the hydrogel formulation that anneals in situ to form a porous covalently stabilized scaffold, wherein endogenous cells infiltrate the porous covalently stabilized scaffold to form a cell matrix over the suture line site of the subject.

2. The method of claim 1, wherein the suture line was an incision.

3.-28. (canceled)

29. The method of claim 1, further comprising increasing a mechanical tensile strength of the suture line characterized by increasing a percent recovery of the suture line, wherein the percent recovery of the suture line is greater than or equal to about 40% increased as compared to a reference suture line sutured without the delivery of the hydrogel formulation.

30. The method of claim 1, further comprising increasing a mechanical tensile strength of the suture line characterized by increasing a percent recovery of the suture line, wherein the percent recovery of the suture line is at least about 20% to about 60% increased as compared to a reference suture line sutured without the delivery of the hydrogel formulation.

31.-70. (canceled)

71. A hydrogel system, comprising:
a) microgel particles comprising a cross-linked 4-arm poly(ethylene glycol) (PEG) vinyl sulfone hydrogel polymer cross-linked with a matrix metalloprotease (MMP)-degradable crosslinker, one or more K-peptides and Q-peptides, and a cell-adhesive peptide, wherein the microgel particles are present in a suspension comprising the microgel particles and water, and the microgel particles are present in the suspension at a volume fraction of at least 90%; and
b) PEG-dithiol, wherein the microgel particles undergo an annealing reaction to form a porous covalently stabilized scaffold after exposure to the PEG-dithiol, wherein the PEG-dithiol is present in the hydrogel system in a molar concentration of at least about 0.02 millimolar (mM) to about 1.0 mM.

72. (canceled)

73. The hydrogel system of claim 71, wherein the PEG-dithiol and the 4-arm PEG vinyl sulfone are present to provide a ratio of thiol to vinyl sulfone of less than about 1.0.

74. The hydrogel system of claim 71, wherein the PEG-dithiol comprises a molecular weight of at least about 3.4 kDa.

75. (canceled)

76. The hydrogel system of claim 71, wherein the microgel particles are spherical.

77. (canceled)

78. The hydrogel system of claim 71, wherein the microgel particles comprise diameters from 5 μm to 1000 μm.

79. The hydrogel system of claim 71, wherein the microgel particles comprise an elastic compressive modulus of at least about 500 Pascals (Pa) before the annealing reaction.

80. (canceled)

81. The hydrogel system of claim 71, wherein the porous covalently stabilized scaffold comprises pores with a median pore diameter of greater than or equal to 5 μm.

82. (canceled)

83. (canceled)

84. The hydrogel system of claim 71, wherein the microgel particles comprise a polydispersity of no more than 0.1.

85. The hydrogel system of claim 84, wherein the polydispersity is calculated based on a standard deviation and mean size of the particles.

86. The hydrogel system of claim 71, wherein the hydrogel formulation further comprises a buffer, wherein the buffer comprises: a phosphate buffer, a 4-(2-hydroxyethyl)-1-piperazineethanesulfonic acid (HEPES) buffer, or an acetate buffer, or any combination thereof.

87. The hydrogel system of claim 71, wherein the annealing reaction comprises a covalent synthesizing reaction.

88. (canceled)

89. The hydrogel system of claim 87, wherein the covalent synthesizing reaction comprises a Michael addition or a pseudo-Michael addition reaction, wherein the vinyl sulfone of the 4-arm PEG vinyl sulfone is a Michael acceptor in the Michael addition or pseudo-Michael addition reaction.

90. The hydrogel system of claim 87, wherein the covalent synthesizing reaction comprises a Michael addition or a pseudo-Michael addition reaction, wherein the thiol of the PEG-dithiol is a Michael donor in the Michael addition or pseudo-Michael addition reaction.

91. A hydrogel formulation comprising the hydrogel system of claim 71 in a suspension, wherein the suspension comprises a buffer.

92.-94. (canceled)

95. The hydrogel formulation of claim 91, wherein the formulation comprises a dose volume of about 0.01 mL to about 20 mL.

96. A delivery device comprising:
a) a body comprising the hydrogel system of claim 71; and
b) an applicator in fluidic communication with the body, wherein the delivery device is sterile, wherein the delivery device is a syringe or a needle.

97. (canceled)
